(19) United States
(12) Patent Application Publication
(10) Pub. No.: US 2007/0299101 A1
(43) Pub. Date: Dec. 27, 2007

(54) Niacin Receptor Agonists, Compositions Containing Such Compounds and Methods of Treatment

(76) Inventors: Steven L. Colletti, Princeton Junction, NJ (US); Richard T. Beresis, Matawan, NJ (US); Weichun Chen, Livingston, NJ (US); James R. Tata, Westfield, NJ (US); Hong C. Shen, West Windsor, NJ (US); Daria M. Marley, Hoboken, NJ (US); Qiaolin Deng, Edison, NJ (US); Jessica L. Frie, Princeton, NJ (US); Fa-Xiang Ding, Staten Island, NY (US)

Correspondence Address: MERCK AND CO., INC, P O BOX 2000, RAHWAY, NJ, 07065-0907, US

(21) Appl. No.: 11/666,966

(22) PCT Filed: Oct. 31, 2005
(86) PCT No.: PCT/US05/39523

§ 371 Date: May 2, 2007

Related U.S. Application Data
(60) Provisional application No. 60/624,816, filed on Nov. 4, 2004.

(51) Int. Cl. A61K 31/47; A61K 31/195; A61K 31/403; A61K 31/41; A61P 3/10; C07C 233/64; C07D 215/02; C07D 257/04; C07D 487/00; C07D 513/00; C07D 471/02; C07D 217/00; C07D 209/56; A61P 9/00; A61K 31/425; A61K 31/428; A61K 31/437

(52) U.S. Cl. 514/292; 514/307; 514/312; 514/366; 514/381; 514/406; 514/411; 514/563; 514/616; 546/146; 546/153; 546/86; 548/151; 548/250; 548/359.1; 548/449; 564/155; 564/180

(57) ABSTRACT

The present invention relates to niacin receptor agonists of formula: (I); as well as pharmaceutically acceptable salts and solvates. The compounds are useful for treating dyslipidemias, and in particular, reducing serum LDL, VLDL and triglycerides, and raising HDL levels. Pharmaceutical compositions and methods of treatment are also included.

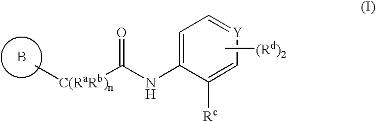

### BACKGROUND OF THE INVENTION

The present invention relates to compounds, compositions and methods of treatment or prevention in a mammal relating to dyslipidemias. Dyslipidemia is a condition wherein serum lipids are abnormal. Elevated cholesterol and low levels of high density lipoprotein (HDL) are associated with a greater-than-normal risk of atherosclerosis and cardiovascular disease. Factors known to affect serum cholesterol include genetic predisposition, diet, body weight, degree of physical activity, age and gender. While cholesterol in normal amounts is a vital building block for cell membranes and essential organic molecules such as steroids and bile acids, cholesterol in excess is known to contribute to cardiovascular disease. For example, cholesterol is a primary component of plaque which collects in coronary arteries, resulting in the cardiovascular disease termed atherosclerosis.

Traditional therapies for reducing cholesterol include medications such as statins (which reduce production of cholesterol by the body). More recently, the value of nutrition and nutritional supplements in reducing blood cholesterol has received significant attention. For example, dietary compounds such as soluble fiber, vitamin E, soy, garlic, omega-3 fatty acids, and niacin have all received significant attention and research funding.

Niacin or nicotinic acid (pyridine-3-carboxylic acid) is a drug that reduces coronary events in clinical trials. It is commonly known for its effect in elevating serum levels of high density lipoproteins (HDL). Importantly, niacin also has a beneficial effect on other lipid profiles. Specifically, it reduces low density lipoproteins (LDL), very low density lipoproteins (VLDL), and triglycerides (TG). However, the clinical use of nicotinic acid is limited by a number of adverse side-effects including cutaneous vasodilation, sometimes called flushing.

Despite the attention focused on traditional and alternative means for controlling serum cholesterol, serum triglycerides, and the like, a significant portion of the population has total cholesterol levels greater than about 200 mg/dL, and are thus candidates for dyslipidemia therapy. There thus remains a need in the art for compounds, compositions and alternative methods of reducing total cholesterol, serum triglycerides, and the like, and raising HDL.

The present invention relates to compounds that have been discovered to have effects in modifying serum lipid levels.

The invention thus provides compositions for effecting reduction in total cholesterol and triglyceride concentrations and raising HDL, in accordance with the methods described.

Consequently one object of the present invention is to provide a nicotinic acid receptor agonist that can be used to treat dyslipidemias, atherosclerosis, diabetes, metabolic syndrome and related conditions while minimizing the adverse effects that are associated with niacin treatment.

Yet another object is to provide a pharmaceutical composition for oral use.

These and other objects will be apparent from the description provided herein.

### SUMMARY OF THE INVENTION

A compound represented by formula I: 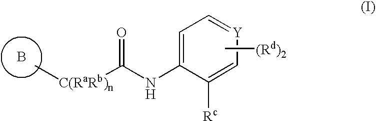
or a pharmaceutically acceptable salt or solvate thereof, wherein:

Y represents C or N;

R^{a} and R^{b} are independently H, C₁₋₃alkyl, haloC₁₋₃alkyl, OC₁₋₃alkyl, haloC₁₋₃alkoxy, OH or F;

R^{c} represents —CO₂H, 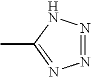
or —C(O)NHSO₂R^{1a};

R^{1a} represents C₁₋₄alkyl or phenyl, said C₁₋₄alkyl or phenyl being optionally substituted with 1-3 substituent groups, 1-3 of which are selected from halo and C₁₋₃alkyl, and 1-2 of which are selected from the group consisting of: OC₁₋₃alkyl, haloC₁₋₃alkyl, haloC₁₋₃alkoxy, OH, NH₂ and NHC₁₋₃alkyl;

each R^{d} independently represents H, halo, methyl, or methyl substituted by 1-3 halo groups;

ring B represents a 10 membered bicyclic aryl, a 9-10 membered bicyclic heteroaryl or a 12-13 membered tricyclic heteroaryl group, 0-1 members of which are O or S and 0-4 members of which are N; said bicyclic aryl or heteroaryl group being optionally substituted with 1-3 groups, 1-3 of which are halo groups and 1-2 of which are selected from the group consisting of:

a) OH; CO₂H; CN; NH₂; S(O)₀₋₂R^{1a};

b) C₁₋₆ alkyl and OC₁₋₆alkyl, said group being optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, Hetcy, CN;

c) Hetcy, NHC₁₋₄alkyl and N(C₁₋₄alkyl)₂, the alkyl portions of which are optionally substituted as set forth in (b) above;

d) Aryl, HAR, C(O)Aryl and C(O) HAR, the Aryl and HAR portions being optionally substituted as set forth in (b) above;

e) C(O)C₁₋₄alkyl and CO₂C₁₋₄alkyl, the alkyl portions of which are optionally substituted as set forth in (b) above; and

f) C(O)NH₂, C(O)NHC₁₋₄alkyl, C(O)N(C₁₋₄alkyl)₂, C(O)NHOC₁₋₄alkyl, C(O)N(C₁₋₄alkyl)(OC₁₋₄alkyl) and C(O)Hetcy, the alkyl portions of which are optionally substituted as set forth in (b) above;

g) NR′C(O)R″, NR′SO₂R″, NR′CO₂R″ and NR′C(O)NR″R′″, wherein:
R′ represents H, C₁₋₃alkyl or haloC₁₋₃alkyl,
R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, CN, ethynyl, Hetcy, Aryl and HAR,
said Hetcy, Aryl and HAR being further optionally substituted with 1-3 halo, C₁₋₄alkyl, C₁₋₄alkoxy, haloC₁₋₄alkyl and haloC₁₋₄alkoxy groups;
   (b) Hetcy, Aryl or HAR, said Hetcy, Aryl and HAR being further optionally substituted with 1-3 halo, C₁₋₄alkyl, C₁₋₄alkoxy, haloC₁₋₄alkyl and haloC₁₋₄alkoxy groups;
and R′″ representing H or R″;

n represents an integer of from 1 to 4, such that (i) when (CR^{a}R^{b})ₙ represents 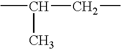
and ring B represents a bicyclic aryl group, said bicyclic aryl group is substituted;

and (ii) when ring B represents a 9-membered heteroaryl group containing one heteroatom, said heteroatom is S or O.

### DETAILED DESCRIPTION OF THE INVENTION

The invention is described herein in detail using the terms defined below unless otherwise specified.

“Alkyl”, as well as other groups having the prefix “alk”, such as alkoxy, alkanoyl and the like, means carbon chains which may be linear, branched, or cyclic, or combinations thereof, containing the indicated number of carbon atoms. If no number is specified, 1-6 carbon atoms are intended for linear and 3-7 carbon atoms for branched alkyl groups. Examples of alkyl groups include methyl, ethyl, propyl, isopropyl, butyl, sec- and tert-butyl, pentyl, hexyl, heptyl, octyl, nonyl and the like. Cycloalkyl is a subset of alkyl; if no number of atoms is specified, 3-7 carbon atoms are intended, forming 1-3 carbocyclic rings that are fused. “Cycloalkyl” also includes monocyclic rings fused to an aryl group in which the point of attachment is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl and the like.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Aryl” (Ar) means mono- and bicyclic aromatic rings containing 6-10 carbon atoms. Examples of aryl include phenyl, naphthyl, indenyl and the like.

“Heteroaryl” (HAR) unless otherwise specified, means mono-, bicyclic and tricyclic aromatic ring systems containing at least one heteroatom selected from O, S, S(O), SO₂ and N, with each ring containing 5 to 6 atoms. HAR groups may contain from 5-14, preferably 5-13 atoms. Examples include, but are not limited to, pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, benzopyrazolyl, benzotriazolyl, furo(2,3-b)pyridyl, benzoxazinyl, tetrahydrohydroquinolinyl, tetrahydroisoquinolinyl., quinolyl, isoquinolyl, indolyl, dihydroindolyl, quinoxalinyl, quinazolinyl, naphthyridinyl, pteridinyl, 2,3-dihydrofuro(2,3-b)pyridyl and the like. Heteroaryl also includes aromatic carbocyclic or heterocyclic groups fused to heterocycles that are non-aromatic or partially aromatic, and optionally containing a carbonyl. Examples of additional heteroaryl groups include indolinyl, dihydrobenzofuranyl, dihydrobenzothiophenyl, dihydrobenzoxazolyl, and aromatic heterocyclic groups fused to cycloalkyl rings. Examples also include the following: 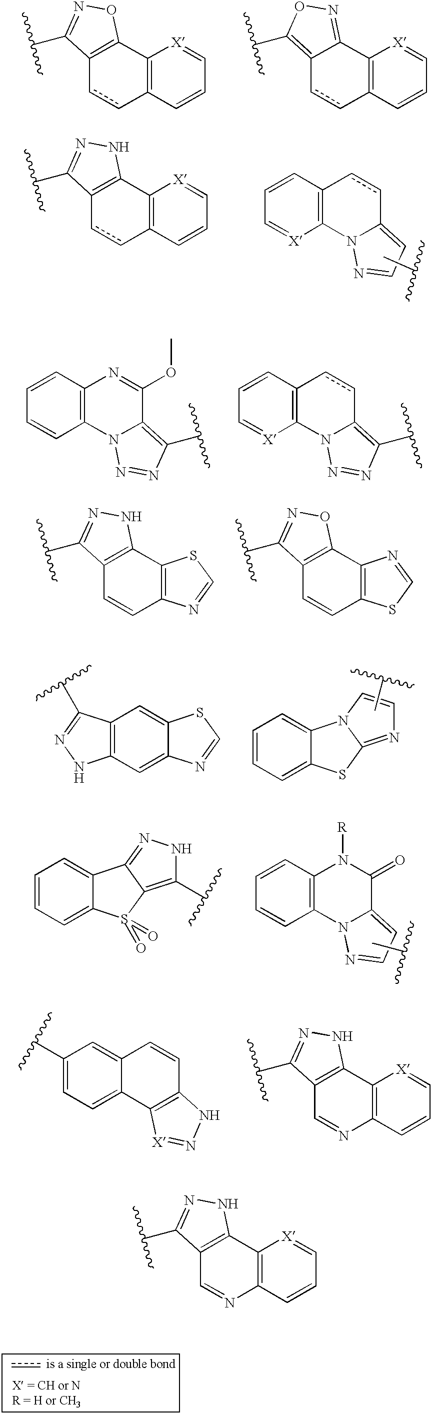
Heteroaryl also includes such groups in charged form, e.g., pyridinium.

“Heterocyclyl” (Hetcy) unless otherwise specified, means mono- and bicyclic saturated rings and ring systems containing at least one heteroatom selected from N, S and O, each of said ring having from 3 to 10 atoms in which the point of attachment may be carbon or nitrogen. Examples of “heterocyclyl” include, but are not limited to, azetidinyl, pyrrolidinyl, piperidinyl, piperazinyl, imidazolidinyl, 2,3-dihydrofuro(2,3-b)pyridyl, tetrahydrofuranyl, benzoxazinyl, 1,4-dioxanyl, tetrahydrohydroquinolinyl, tetrahydroisoquinolinyl, dihydroindolyl, morpholinyl, thiomorpholinyl, tetrahydrothienyl and the like. The term also includes partially unsaturated monocyclic rings that are not aromatic, such as 2- or 4-pyridones attached through the nitrogen or N-substituted-(1H,3H)-pyrimidine-2,4-diones (N-substituted uracils). Heterocyclyl moreover includes such moieties in charged form, e.g., piperidinium.

“Halogen” (Halo) includes fluorine, chlorine, bromine and iodine.

The phrase “in the absence of substantial flushing” refers to the side effect that is often seen when nicotinic acid is administered in therapeutic amounts. The flushing effect of nicotinic acid usually becomes less frequent and less severe as the patient develops tolerance to the drug at therapeutic doses, but the flushing effect still occurs to some extent and can be transient. Thus, “in the absence of substantial flushing” refers to the reduced severity of flushing when it occurs, or fewer flushing events than would otherwise occur. Preferably, the incidence of flushing (relative to niacin) is reduced by at least about a third, more preferably the incidence is reduced by half, and most preferably, the flushing incidence is reduced by about two thirds or more. Likewise, the severity (relative to niacin) is preferably reduced by at least about a third, more preferably by at least half, and most preferably by at least about two thirds. Clearly a one hundred percent reduction in flushing incidence and severity is most preferable, but is not required.

One aspect of the invention relates to compounds of formula I: 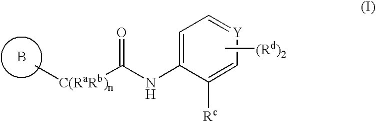
or a pharmaceutically acceptable salt or solvate thereof, wherein:

Y represents C or N;

R^{a} and R^{b} are independently H, C₁₋₃alkyl, haloC₁₋₃alkyl, OC₁₋₃alkyl, haloC₁₋₃alkoxy, OH or F;

R^{c} represents —CO₂H, 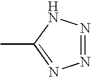
or —C(O)NHSO₂R^{1a};

R^{1a} represents C₁₋₄alkyl or phenyl, said C₁₋₄alkyl or phenyl being optionally substituted with 1-3 substituent groups, 1-3 of which are selected from halo and C₁₋₃alkyl, and 1-2 of which are selected from the group consisting of: OC₁₋₃alkyl, haloC₁₋₃alkyl, haloC₁₋₃alkoxy, OH, NH₂ and NHC₁₋₃alkyl;

each R^{d} independently represents H, halo, methyl, or methyl substituted by 1-3 halo groups;

ring B represents a 10 membered bicyclic aryl, a 9-10 membered bicyclic heteroaryl or a 12-13 membered tricyclic heteroaryl group, 0-1 members of which are O or S and 0-4 members of which are N; said bicyclic aryl or heteroaryl group being optionally substituted with 1-3 groups, 1-3 of which are halo groups and 1-2 of which are selected from the group consisting of:

a) OH; CO₂H; CN; NH₂; S(O)₀₋₂R^{1a};

b) C₁₋₆ alkyl and OC₁₋₆alkyl, said group being optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, Hetcy, CN;

c) Hetcy, NHC₁₋₄alkyl and N(C₁₋₄alkyl)₂, the alkyl portions of which are optionally substituted as set forth in (b) above;

d) Aryl, HAR, C(O)Aryl and C(O) HAR, the Aryl and HAR portions being optionally substituted as set forth in (b) above;

e) C(O)C₁₋₄alkyl and CO₂C₁₋₄alkyl, the alkyl portions of which are optionally substituted as set forth in (b) above; and

f) C(O)NH₂, C(O)NHC₁₋₄alkyl, C(O)N(C₁₋₄alkyl)₂, C(O)NHOC₁₋₄alkyl, C(O)N(C₁₋₄alkyl)(OC₁₋₄alkyl) and C(O)Hetcy, the alkyl portions of which are optionally substituted as set forth in (b) above;

g) NR′C(O)R″, NR′SO₂R″, NR′CO₂R″ and NR′C(O)NR″R′″, wherein:
R′ represents H, C₁₋₃alkyl or haloC₁₋₃alkyl,
R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, CN, ethynyl, Hetcy, Aryl and HAR,
said Hetcy, Aryl and HAR being further optionally substituted with 1-3 halo, C₁₋₄alkyl, C₁₋₄alkoxy, haloC₁₋₄alkyl and haloC₁₋₄alkoxy groups;
   (b) Hetcy, Aryl or HAR, said Hetcy, Aryl and HAR being further optionally substituted with 1-3 halo, C₁₋₄alkyl, C₁₋₄alkoxy, haloC₁₋₄alkyl and haloC₁₋₄alkoxy groups;
and R′″ representing H or R″;

n represents an integer of from 1 to 4, such that (i) when (CR^{a}R^{b}), represents 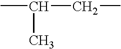
and ring B represents a bicyclic aryl group, said bicyclic aryl group is substituted;

and (ii) when ring B represents a 9-membered heteroaryl group containing one heteroatom, said heteroatom is S or O.

An aspect of the invention that is of interest relates to compounds of formula I or a pharmaceutically acceptable salt or solvate thereof wherein ring B represents naphthyl or a bicyclic 9-10 membered heteroaryl group containing 1-2 heteroatoms, 0-1 of which is O or S, and 1-2 of which are nitrogen. Within this aspect of the invention, all other variables are as originally defined.

More particularly, an aspect of the invention that is of interest relates to compounds of formula I or a pharmaceutically acceptable salt or solvate thereof wherein ring B represents naphthyl, quinolinyl, isoquinolinyl or benzothiazolyl. Within this aspect of the invention, all other variables are as originally defined.

Even more particularly, an aspect of the invention that is of interest relates to compounds of formula I or a pharmaceutically acceptable salt or solvate thereof wherein ring B represents 1- or 2-naphthyl, 2-, 6- or 7-quinolinyl, 5-, 6- or 7-isoquinolinyl, or 5- or 6-benzothiazolyl. Within this aspect of the invention, all other variables are as originally defined.

An even more particular aspect of the invention that is of interest relates to compounds of formula I or a pharmaceutically acceptable salt or solvate thereof wherein B represents naphthyl or quinolinyl. Within this aspect of the invention, all other variables are as originally defined.

An even more particular aspect of the invention that is of more interest relates to compounds of formula I or a pharmaceutically acceptable salt or solvate thereof wherein B represents naphthyl. Within this aspect of the invention, all other variables are as originally defined.

Another even more particular aspect of the invention that is of more interest relates to compounds of formula I or a pharmaceutically acceptable salt or solvate thereof wherein B represents quinolinyl. Within this aspect of the invention, all other variables are as originally defined.

An even more particular aspect of the invention that is of interest relates to compounds of formula I or a pharmaceutically acceptable salt or solvate thereof wherein B represents isoquinolinyl. Within this aspect of the invention, all other variables are as originally defined.

Another aspect of the invention relates to compounds of formula I or a pharmaceutically acceptable salt or solvate thereof wherein:

Ring B is selected from naphthyl, quinolinyl, isoquinolinyl and benzothiazolyl, optionally substituted with 1-3 groups, 1-3 of which are halo groups selected from Cl and F, and 1-2 groups are selected from:

a) OH; CO₂H; CN; NH₂;

b) C₁₋₄ alkyl and OC₁₋₄alkyl, said group being optionally substituted with 1-3 groups, 1-3 of which are halo selected from Cl and F, and I of which is selected from: OH, CO₂H, CO₂C₁₋₂alkyl, CO₂C₁₋₂haloalkyl wherein halo is selected from Cl and F, OCO₂C₁₋₄alkyl, NH₂, NHC₁₋₄alkyl, N(C₁₋₄alkyl)₂, Hetcy and CN;

c) Hetcy, NHC₁₋₄alkyl and N(C₁₋₄alkyl)₂, the alkyl portions of which are optionally substituted as set forth in (b) above;

d) C(O)NH₂, C(O)NHC₁₋₄alkyl and C(O)N(C₁₋₂alkyl)₂, the alkyl portions of which are optionally substituted as set forth in (b) above;

e) NR′C(O)R″, NR′SO₂R″, NR′CO₂R″ and NR′C(O)NR″R′″ wherein:
R′ represents H, C₁₋₃alkyl or haloC₁₋₃alkyl wherein halo is selected from Cl and F,
R″ represents (a) C₁₋₈alkyl optionally substituted with 1-4 groups, 0-4 of which are halo selected from Cl and F, and 0-1 of which are selected from the group consisting of: OC₁₋₄alkyl, OH, CO₂H, CO₂C₁₋₄alkyl, CO₂C₁₋₄haloalkyl, OCO₂C₁₋₄alkyl, NH₂, NHC₁₋₄alkyl, N(C₁₋₂alkyl)₂, CN, ethynyl, Hetcy, Aryl and HAR,
said Hetcy, Aryl and HAR being further optionally substituted with 1-3 halo groups, C₁₋₄alkyl, C₁₋₄alkoxy, haloC₁₋₄alkyl and haloC₁₋₄alkoxy groups, the halo and halo portions of which are selected from Cl and F;
   (b) Hetcy, Aryl or HAR, said Hetcy, Aryl and HAR being further optionally substituted with 1-3 halo, C₁₋₄alkyl, C₁₋₄alkoxy, haloC₁₋₄alkyl and haloC₁₋₄alkoxy groups, the halo and halo portions of which are selected from Cl and F;
and R′″ representing H or R″. Within this aspect of the invention, all other variables are as originally defined.

Even more particularly, an aspect of the invention that is of interest relates to compounds of formula I or a pharmaceutically acceptable salt or solvate thereof wherein:

Ring B is naphthyl optionally substituted with 1-2 halo groups selected from Cl and F, and 0-1 group selected from:

a) OH;

b) C₁₋₄ alkyl and OC₁₋₄alkyl, said group being optionally substituted with 1-3 groups, 1-3 of which are halo selected from Cl and F;

c) NR′C(O)R″, NR′SO₂R″, NR′CO₂R″ and NR′C(O)NR″R′″ wherein:
R′ represents H, C₁₋₃alkyl or haloC₁₋₃alkyl wherein halo is selected from Cl and F,
R″ represents (a) C₁₋₈alkyl optionally substituted with 1-4 groups, 0-4 of which are halo selected from Cl and F, and 0-1 of which are selected from the group consisting of: OC₁₋₄alkyl, OH, CO₂H, CO₂C₁₋₄alkyl, CO₂C₁₋₄haloalkyl, OCO₂C₁₋₄alkyl, NH₂, NHC₁₋₄alkyl, N(C₁₋₂alkyl)₂, CN, ethynyl, Hetcy, Aryl and HAR,
said Hetcy, Aryl and HAR being further optionally substituted with 1-3 halo groups, C₁₋₄alkyl, C₁₋₄alkoxy, haloC₁₋₄alkyl and haloC₁₋₄alkoxy groups, the halo and halo portions of which are selected from Cl and F;
   (b) Hetcy, Aryl or HAR, said Hetcy, Aryl and HAR being further optionally substituted with 1-3 halo, C₁₋₄alkyl, C₁₋₄alkoxy, haloC₁₋₄alkyl and haloC₁₋₄alkoxy groups, the halo and halo portions of which are selected from Cl and F;
and R′″ representing H or R″. Within this aspect of the invention, all other variables are as originally defined.

Another aspect of the invention that is of interest relates to compounds of formula I or a pharmaceutically acceptable salt or solvate thereof wherein Y represents C. Within this aspect of the invention, all other variables are as originally defined.

Another aspect of the invention that is of interest relates to compounds of formula I or a pharmaceutically acceptable salt or solvate thereof wherein Y represents N. Within this aspect of the invention, all other variables are as originally defined.

Another aspect of the invention that is of interest relates to compounds of formula I or a pharmaceutically acceptable salt or solvate thereof wherein n represents 2, 3 or 4. Within this aspect of the invention, all other variables are as originally defined.

In particular, another aspect of the invention that is of interest relates to compounds of formula I or a pharmaceutically acceptable salt or solvate thereof wherein n represents an integer 2, 3 or 4, and one or both of R^{a} and R^{b} represents H or CH₃, and the remaining R^{a} and R^{b} groups, if any, represent H. Within this aspect of the invention, all other variables are as originally defined.

Another aspect of the invention that is of interest relates to compounds of formula I or a pharmaceutically acceptable salt or solvate thereof wherein R^{c}C represents CO₂H. Within this aspect of the invention, all other variables are as originally defined.

Another aspect of the invention that is of interest relates to compounds of formula I or a pharmaceutically acceptable salt or solvate thereof wherein R^{c} represents tetrazolyl. Within this aspect of the invention, all other variables are as originally defined.

Another aspect of the invention that is of interest relates to compounds of formula I or a pharmaceutically acceptable salt or solvate thereof wherein R^{d} represents H or halo. Within this aspect of the invention, all other variables are as originally defined.

More particularly, an aspect of the invention that is of interest relates to compounds of formula I or a pharmaceutically acceptable salt or solvate thereof wherein R^{d} represents H. Within this aspect of the invention, all other variables are as originally defined.

More particularly, an aspect of the invention that is of interest relates to compounds of formula I or a pharmaceutically acceptable salt or solvate thereof wherein R^{d} represents halo, and in particular, F. Within this aspect of the invention, all other variables are as originally defined.

Another aspect of the invention that is of interest relates to compounds of formula I or a pharmaceutically acceptable salt or solveate thereof wherein one of R^{a} and R^{b} is selected from the group consisting of: C₁₋₃alkyl, haloC₁₋₃alkyl, OC₁₋₃alkyl, haloC₁₋₃alkoxy, OH and F, and the other is selected from the group consisting of: H, C₁₋₃alkyl, haloC₁₋₃alkyl, OC₁₋₃alkyl, haloC₁₋₃alkoxy, OH and F. Within this aspect of the invention, all other variables are as originally defined.

Another aspect of the invention that is of particular interest relates to compounds of formula I or a pharmaceutically acceptable salt or solveate thereof wherein one of R^{a} and R^{b} is C₁₋₃alkyl. Within this aspect of the invention, all other variables are as originally defined.

Even more particularly, another aspect of the invention that is of interest relates to compounds of formula I or a pharmaceutically acceptable salt or solveate thereof wherein one of R^{a} and R^{b} is methyl. Within this aspect of the invention, all other variables are as originally defined.

Another aspect of the invention that is of interest relates to compounds of formula I or a pharmaceutically acceptable salt or solvate thereof wherein at least one R^{d} group is selected from the group consisting of: halo, methyl and methyl substituted with 1-3 halo groups, and is located ortho or meta to R^{c}. Within this aspect of the invention, all other variables are as originally defined.

Another aspect of the invention that is of interest relates to compounds of formula I or a pharmaceutically acceptable salt or solvate thereof wherein ring B is substituted with from 1-3 groups, 1-3 of which are halo atoms, and 1-2 of which are selected from OH and NH₂. Within this aspect of the invention, all other variables are as originally defined.

Another aspect of the invention that is of interest relates to compounds of formula I or a pharmaceutically acceptable salt or solvate thereof wherein ring B represents a 12-13 membered tricyclic heteroaryl group, 0-1 members of which are O or S, and 0-4 of which are N, said group being optionally substituted with 1-3 groups, 1-3 of which are halo atoms and 1-2 of which are selected from the group consisting of:

a) OH; CO₂H; CN; NH₂; S(O)₀₋₂R^{1a};

b) C₁₋₆ alkyl and OC₁₋₆alkyl, said group being optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, Hetcy, CN;

c) Hetcy, NHC₁₋₄alkyl and N(C₁₋₄alkyl)₂, the alkyl portions of which are optionally substituted as set forth in (b) above;

d) Aryl, HAR, C(O)Aryl and C(O) HAR, the Aryl and HAR portions being optionally substituted as set forth in (b) above;

e) C(O)C₁₋₄alkyl and CO₂C₁₋₄alkyl, the alkyl portions of which are optionally substituted as set forth in (b) above; and

f) C(O)NH₂, C(O)NHC₁₋₄alkyl, C(O)N(C₁₋₄alkyl)₂, C(O)NHOC₁₋₄alkyl, C(O)N(C₁₋₄alkyl)(OC₁₋₄alkyl) and C(O)Hetcy, the alkyl portions of which are optionally substituted as set forth in (b) above;

g) NR′C(O)R″, NR′SO₂R″, NR′CO₂R″ and NR′C(O)NR″R′″ wherein:
R′ represents H, C₁₋₃alkyl or haloC₁₋₃alkyl,
R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, CN, ethynyl, Hetcy, Aryl and HAR,
said Hetcy, Aryl and HAR being further optionally substituted with 1-3 halo, C₁₋₄alkyl, C₁₋₄alkoxy, haloC₁₋₄alkyl and haloC₁₋₄alkoxy groups;
   (b) Hetcy, Aryl or HAR, said Hetcy, Aryl and HAR being further optionally substituted with 1-3 halo, C₁₋₄alkyl, C₁₋₄alkoxy, haloC₁₋₄alkyl and haloC₁₋₄alkoxy groups;
and R′″ representing H or R″.
   Within this aspect of the invention, all other variables are as originally defined.

More particularly, an aspect of the invention that is of interest relates to compounds of formula I or a pharmaceutically acceptable salt or solvate thereof wherein ring B represents a member selected from the group consisting of: 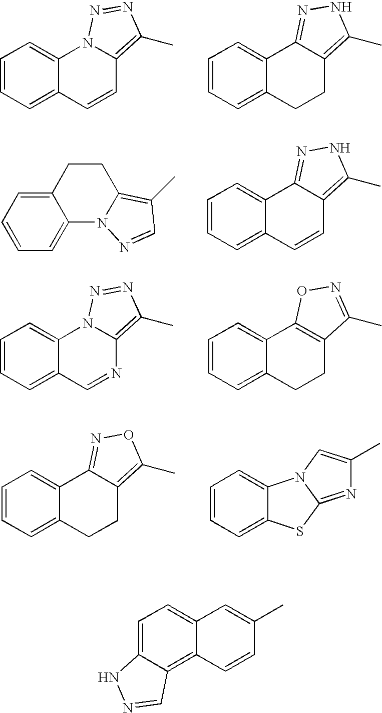
Within this aspect of the invention, all other variables are as originally defined.

Examples of compounds falling within the present invention are set forth below in Table 1:

TABLE 1

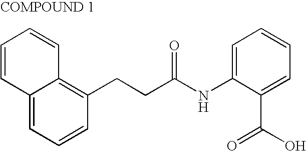

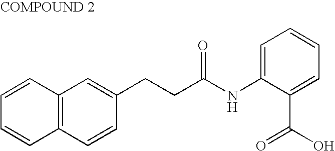

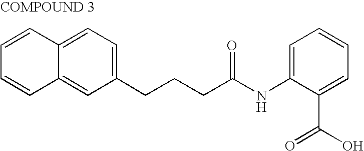

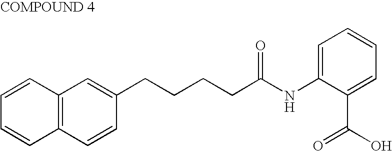

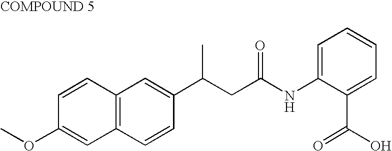

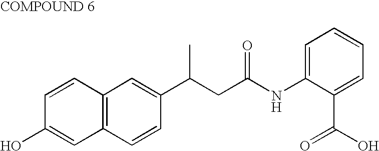

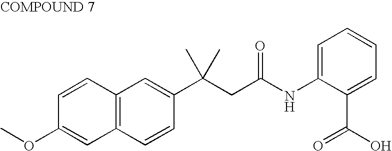

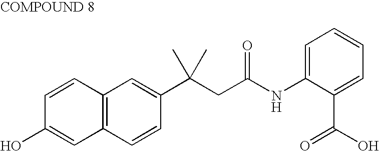

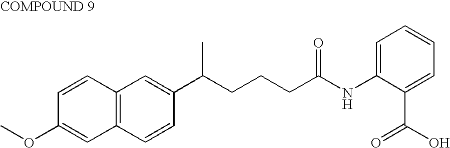

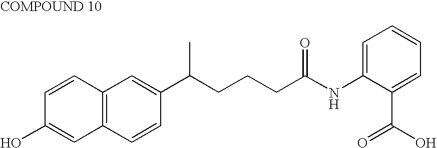

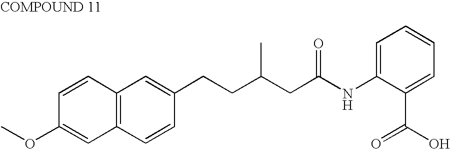

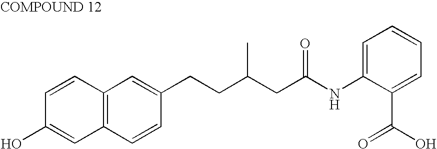

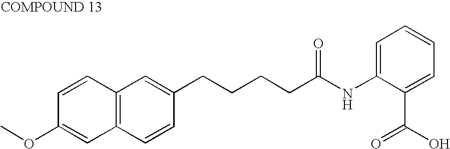

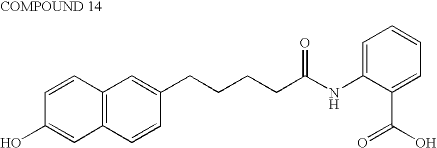

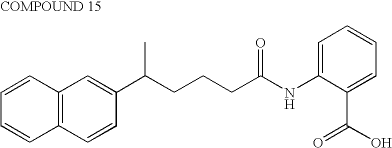

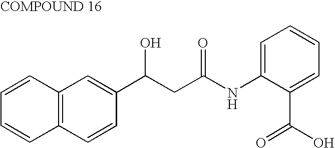

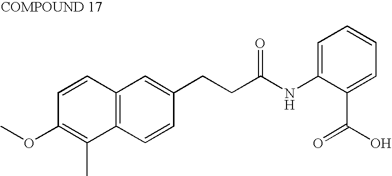

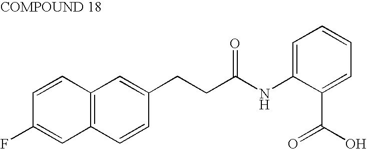

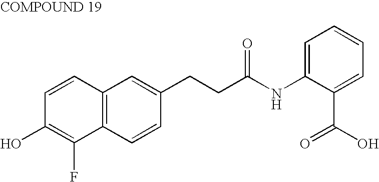

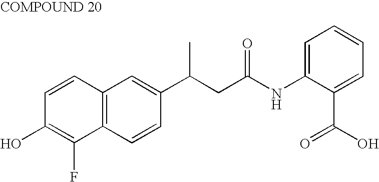

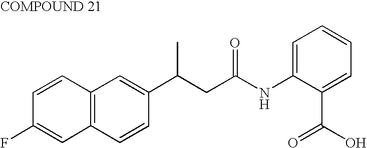

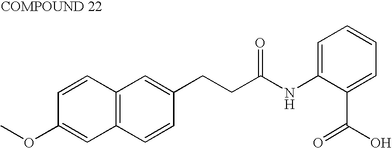

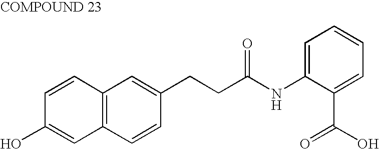

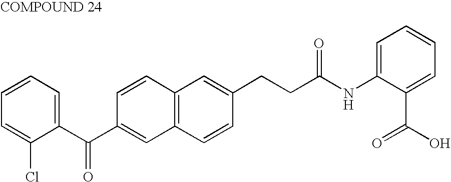

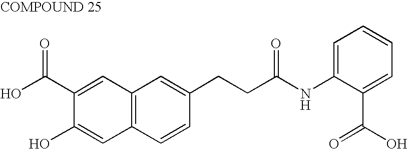

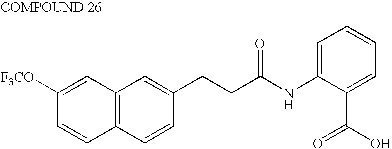

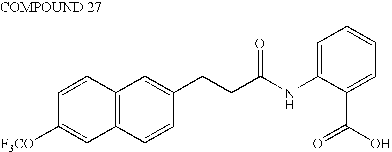

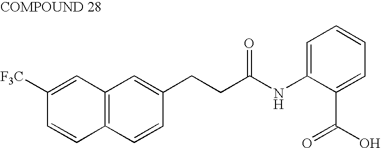

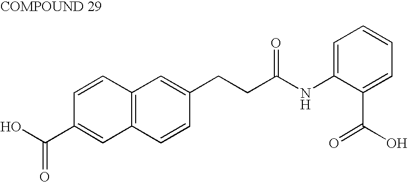

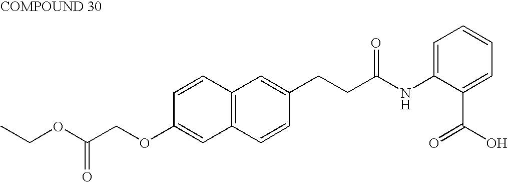

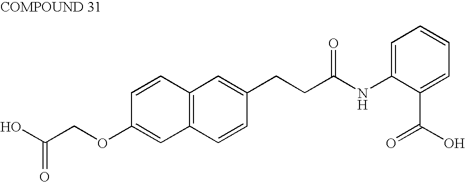

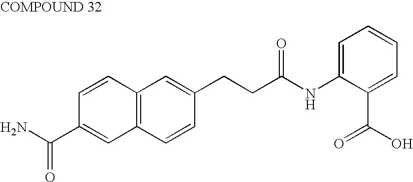

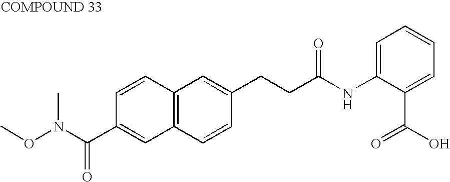

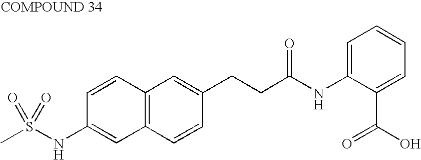

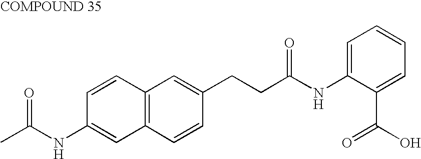

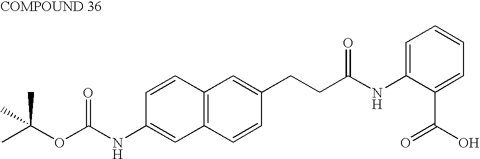

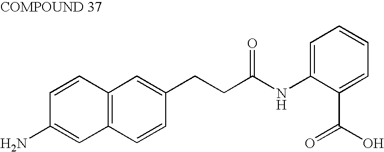

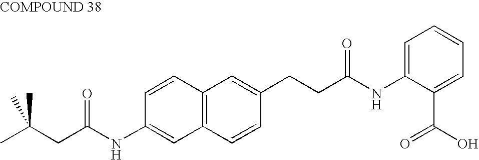

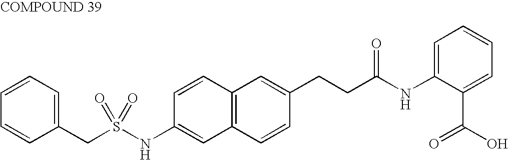

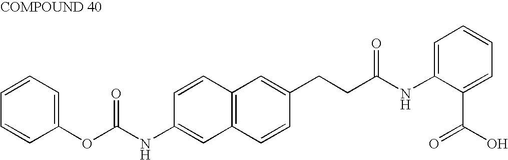

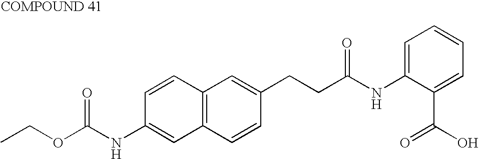

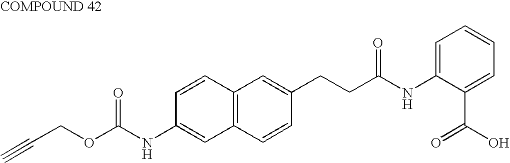

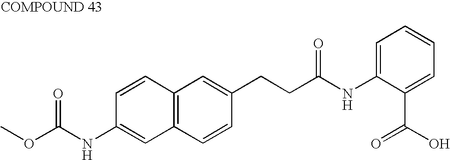

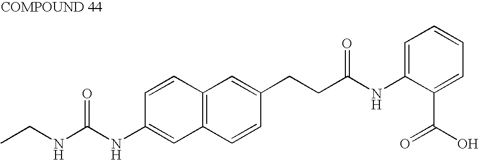

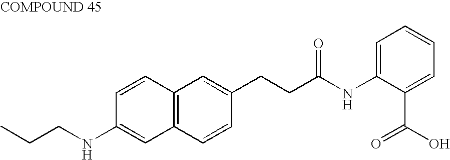

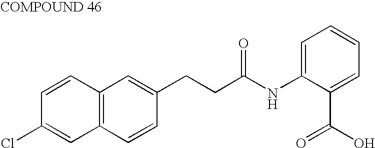

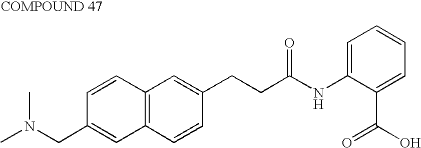

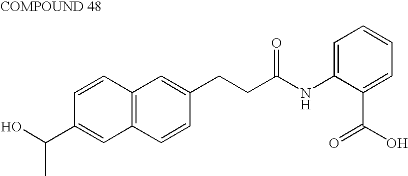

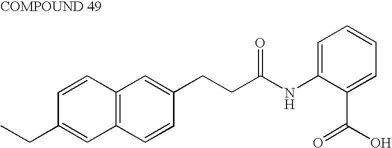

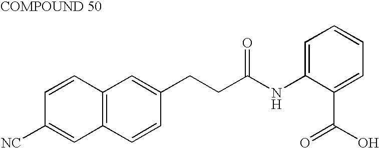

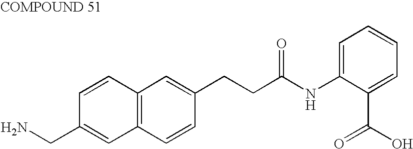

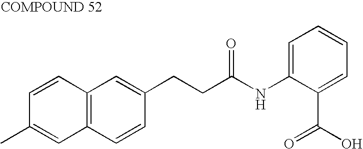

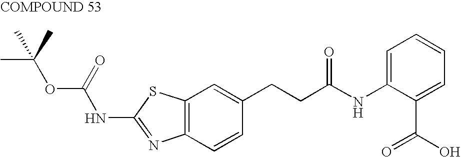

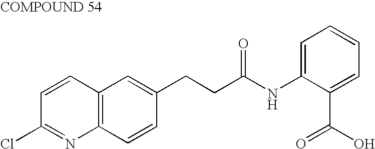

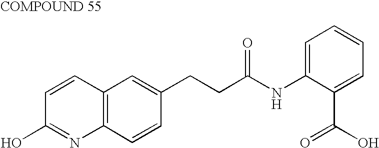

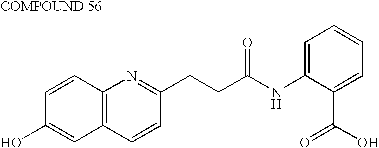

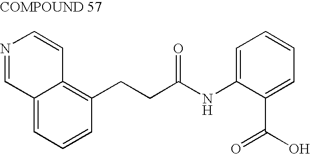

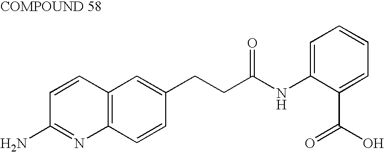

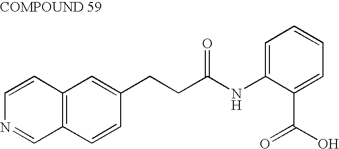

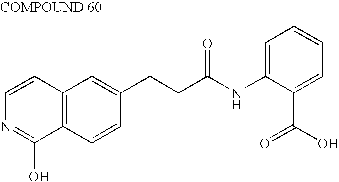

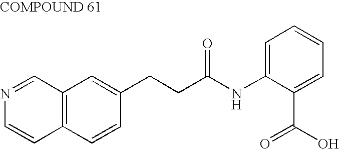

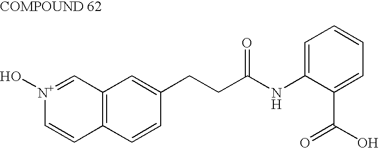

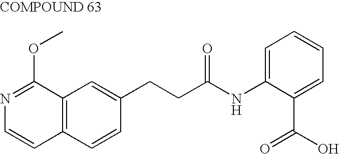

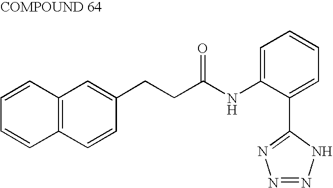

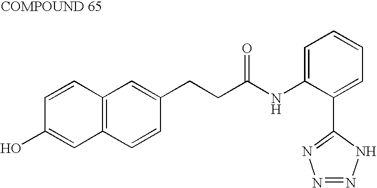

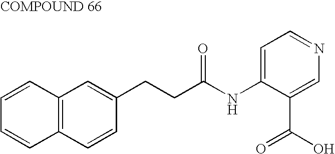

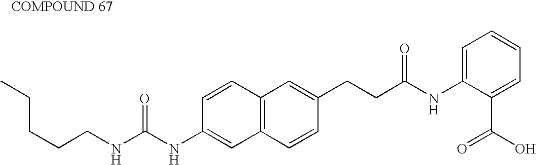

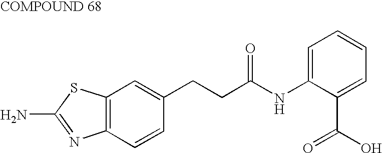

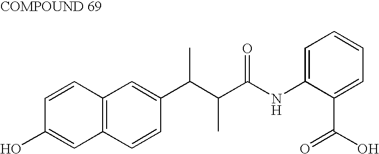

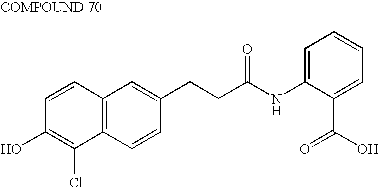

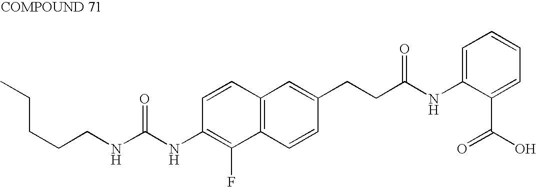

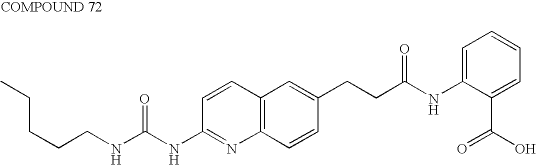

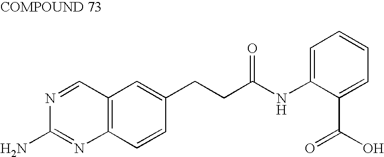

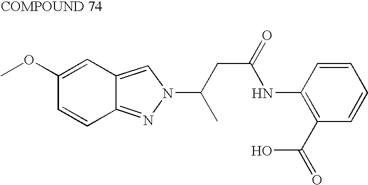

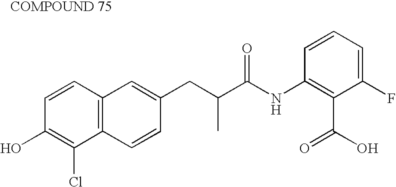

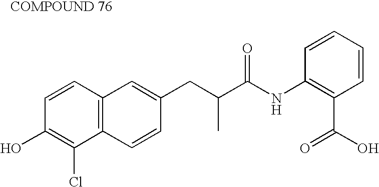

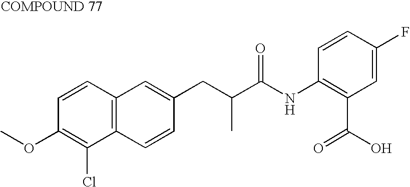

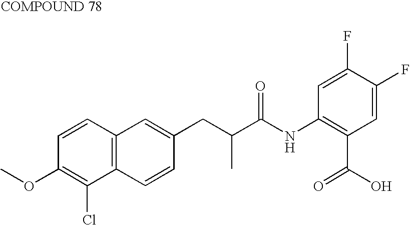

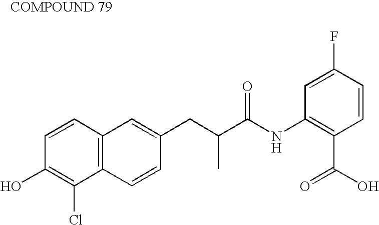

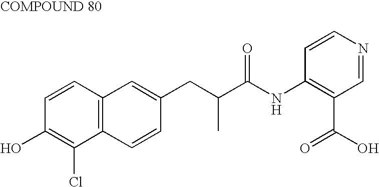

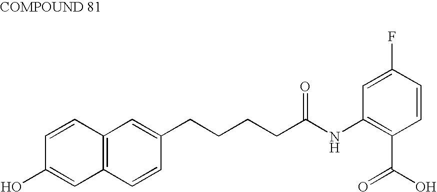

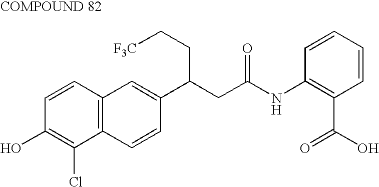

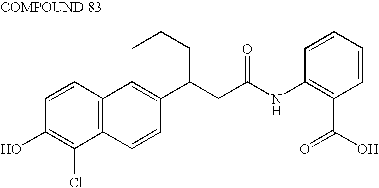

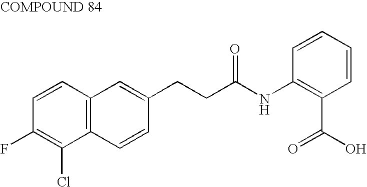

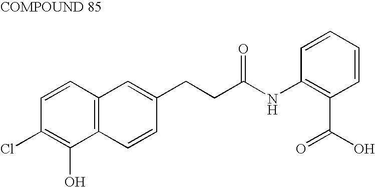

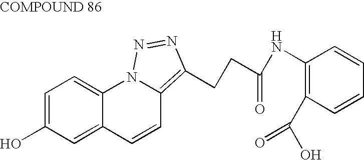

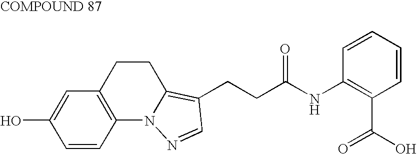

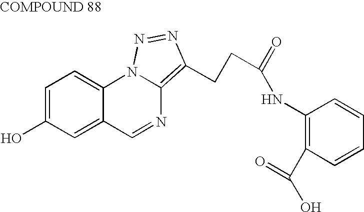

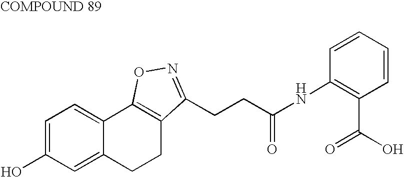

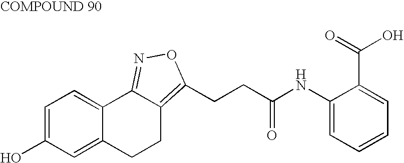

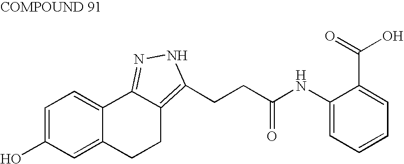

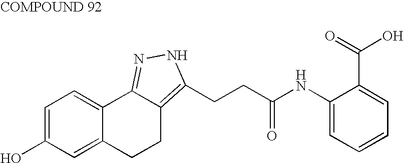

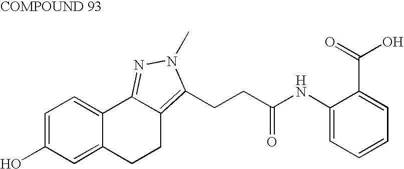

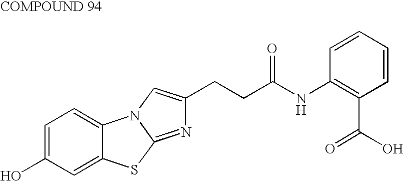

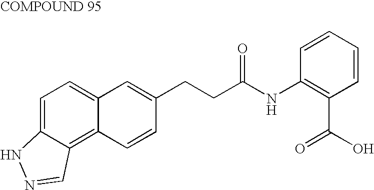

Pharmaceutically acceptable salts and solvates thereof are included as well.

Many of the compounds of formula I contain asymmetric centers and can thus occur as racemates and racemic mixtures, single enantiomers, diastereomeric mixtures and individual diastereomers. All such isomeric forms are included.

Moreover, chiral compounds possessing one stereocenter of general formula I, may be resolved into their enantiomers in the presence of a chiral environment using methods known to those skilled in the art. Chiral compounds possessing more than one stereocenter may be separated into their diastereomers in an achiral environment on the basis of their physical properties using methods known to those skilled in the art. Single diastereomers that are obtained in racemic form may be resolved into their enantiomers as described above.

If desired, racemic mixtures of compounds may be separated so that individual enantiomers are isolated. The separation can be carried out by methods well known in the art, such as the coupling of a racemic mixture of compounds of Formula I to an enantiomerically pure compound to form a diastereomeric mixture, which is then separated into individual diastereomers by standard methods, such as fractional crystallization or chromatography. The coupling reaction is often the formation of salts using an enantiomerically pure acid or base. The diasteromeric derivatives may then be converted to substantially pure enantiomers by cleaving the added chiral residue from the diastereomeric compound.

The racemic mixture of the compounds of Formula I can also be separated directly by chromatographic methods utilizing chiral stationary phases, which methods are well known in the art.

Alternatively, enantiomers of compounds of the general Formula I may be obtained by stereoselective synthesis using optically pure starting materials or reagents.

Some of the compounds described herein exist as tautomers, which have different points of attachment for hydrogen accompanied by one or more double bond shifts. For example, a ketone and its enol form are keto-enol tautomers. Or for example, a 2-hydroxyquinoline can reside in the tautomeric 2-quinolone form. The individual tautomers as well as mixtures thereof are included.

Dosing Information

The dosages of compounds of formula I or a pharmaceutically acceptable salt or solvate thereof vary within wide limits. The specific dosage regimen and levels for any particular patient will depend upon a variety of factors including the age, body weight, general health, sex, diet, time of administration, route of administration, rate of excretion, drug combination and the severity of the patient's condition. Consideration of these factors is well within the purview of the ordinarily skilled clinician for the purpose of determining the therapeutically effective or prophylactically effective dosage amount needed to prevent, counter, or arrest the progress of the condition. Generally, the compounds will be administered in amounts ranging from as low as about 0.01 mg/day to as high as about 2000 mg/day, in single or divided doses. A representative dosage is about 0.1 mg/day to about 1 g/day. Lower dosages can be used initially, and dosages increased to further minimize any untoward effects. It is expected that the compounds described herein will be administered on a daily basis for a length of time appropriate to treat or prevent the medical condition relevant to the patient, including a course of therapy lasting months, years or the life of the patient.

Combination Therapy

One or more additional active agents may be administered with the compounds described herein. The additional active agent or agents can be lipid modifying compounds or agents having other pharmaceutical activities, or agents that have both lipid-modifying effects and other pharmaceutical activities. Examples of additional active agents which may be employed include but are not limited to HMG-CoA reductase inhibitors, which include statins in their lactonized or dihydroxy open acid forms and pharmaceutically acceptable salts and esters thereof, including but not limited to lovastatin (see U.S. Pat. No. 4,342,767), simvastatin (see U.S. Pat. No. 4,444,784), dihydroxy open-acid simvastatin, particularly the ammonium or calcium salts thereof, pravastatin, particularly the sodium salt thereof (see U.S. Pat. No. 4,346,227), fluvastatin particularly the sodium salt thereof (see U.S. Pat. No. 5,354,772), atorvastatin, particularly the calcium salt thereof (see U.S. Pat. No. 5,273,995), pitavastatin also referred to as NK-104 (see PCT international publication number WO 97/23200) and rosuvastatin, also known as CRESTOR®; see U.S. Pat. No. 5,260,440); HMG-CoA synthase inhibitors; squalene epoxidase inhibitors; squalene synthetase inhibitors (also known as squalene synthase inhibitors), acyl-coenzyme A: cholesterol acyltransferase (ACAT) inhibitors including selective inhibitors of ACAT-1 or ACAT-2 as well as dual inhibitors of ACAT-1 and -2; microsomal triglyceride transfer protein (MTP) inhibitors; endothelial lipase inhibitors; bile acid sequestrants; LDL receptor inducers; platelet aggregation inhibitors, for example glycoprotein IIb/IIIa fibrinogen receptor antagonists and aspirin; human peroxisome proliferator activated receptor gamma (PPARγ) agonists including the compounds commonly referred to as glitazones for example pioglitazone and rosiglitazone and, including those compounds included within the structural class known as thiazolidine diones as well as those PPARγ agonists outside the thiazolidine dione structural class; PPARα agonists such as clofibrate, fenofibrate including micronized fenofibrate, and gemfibrozil; PPAR dual α/γ agonists; vitamin B₆ (also known as pyridoxine) and the pharmaceutically acceptable salts thereof such as the HCl salt; vitamin B₁₂ (also known as cyanocobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such as losartan; angiotensin converting enzyme inhibitors such as enalapril and captopril; renin inhibitors, calcium channel blockers such as nifedipine and diltiazem; endothelin antagonists; agents that enhance ABCA1 gene expression; cholesteryl ester transfer protein (CETP) inhibiting compounds, 5-lipoxygenase activating protein (FLAP) inhibiting compounds, 5-lipoxygenase (5-LO) inhibiting compounds, farnesoid X receptor (FXR) ligands including both antagonists and agonists; Liver X Receptor (LXR)-alpha ligands, LXR-beta ligands, bisphosphonate compounds such as alendronate sodium; cyclooxygenase-2 inhibitors such as rofecoxib and celecoxib; and compounds that attenuate vascular inflammation.

Cholesterol absorption inhibitors can also be used in the present invention. Such compounds block the movement of cholesterol from the intestinal lumen into enterocytes of the small intestinal wall, thus reducing serum cholesterol levels. Examples of cholesterol absorption inhibitors are described in U.S. Pat. Nos. 5,846,966, 5,631,365, 5,767,115, 6,133,001, 5,886,171, 5,856,473, 5,756,470, 5,739,321, 5,919,672, and in PCT application Nos. WO 00/63703, WO 00/60107, WO 00/38725, WO 00/34240, WO 00/20623, WO 97/45406, WO 97/16424, WO 97/16455, and WO 95/08532. The most notable cholesterol absorption inhibitor is ezetimibe, also known as 1-(4-fluorophenyl)-3(R)-[3 (S)-(4-fluorophenyl)-3-hydroxypropyl)]-4(S)-(4-hydroxyphenyl)-2-azetidinone, described in U.S. Pat. Nos. 5,767,115 and 5,846,966.

Therapeutically effective amounts of cholesterol absorption inhibitors include dosages of from about 0.01 mg/kg to about 30 mg/kg of body weight per day, preferably about 0.1 mg/kg to about 15 mg/kg.

For diabetic patients, the compounds used in the present invention can be administered with conventional diabetic medications. For example, a diabetic patient receiving treatment as described herein may also be taking insulin or an oral antidiabetic medication. One example of an oral antidiabetic medication useful herein is metformin.

In the event that these niacin receptor agonists induce some degree of vasodilation, it is understood that the compounds of formula I may be co-dosed with a vasodilation suppressing agent. Consequently, one aspect of the methods described herein relates to the use of a compound of formula I or a pharmaceutically acceptable salt or solvate thereof in combination with a compound that reduces flushing. Conventional compounds such as aspirin, ibuprofen, naproxen, indomethacin, other NSAIDs, COX-2 selective inhibitors and the like are useful in this regard, at conventional doses. Alternatively, DP antagonists are useful as well. Doses of the DP receptor antagonist and selectivity are such that the DP antagonist selectively modulates the DP receptor without substantially modulating the CRTH2 receptor. In particular, the DP receptor antagonist ideally has an affinity at the DP receptor (i.e., Kᵢ) that is at least about 10 times higher (a numerically lower Kᵢ value) than the affinity at the CRTH2 receptor. Any compound that selectively interacts with DP according to these guidelines is deemed “DP selective”.

Dosages for DP antagonists as described herein, that are useful for reducing or preventing the flushing effect in mammalian patients, particularly humans, include dosages ranging from as low as about 0.01 mg/day to as high as about 100 mg/day, administered in single or divided daily doses. Preferably the dosages are from about 0.1 mg/day to as high as about 1.0 g/day, in single or divided daily doses.

Examples of compounds that are particularly useful for selectively antagonizing DP receptors and suppressing the flushing effect include the following: 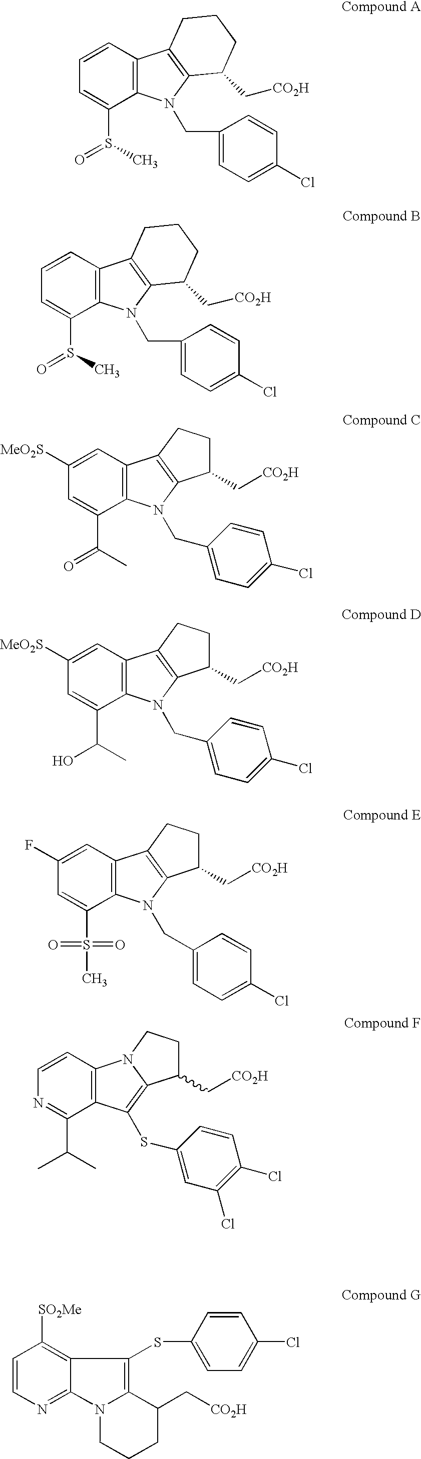 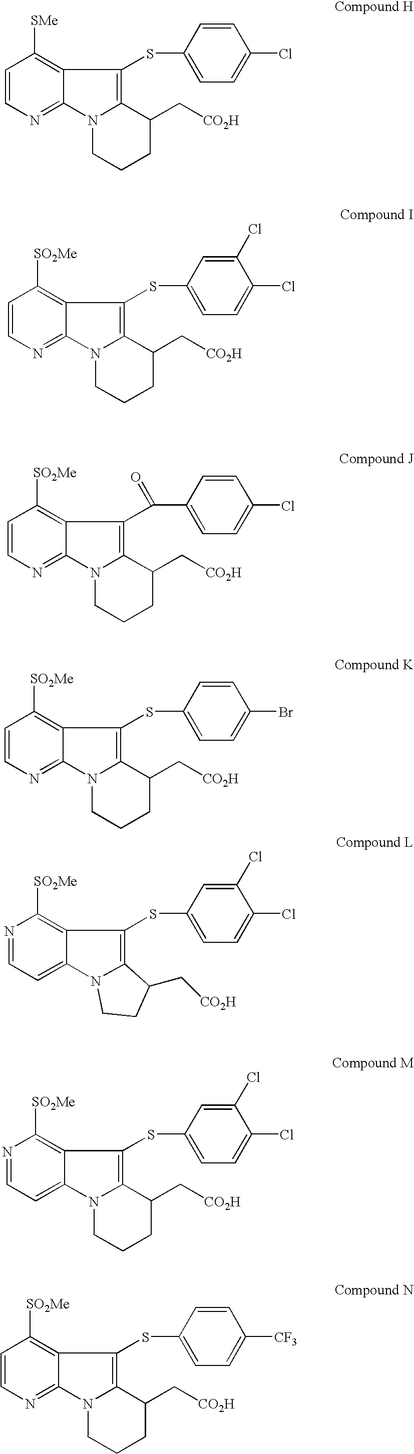 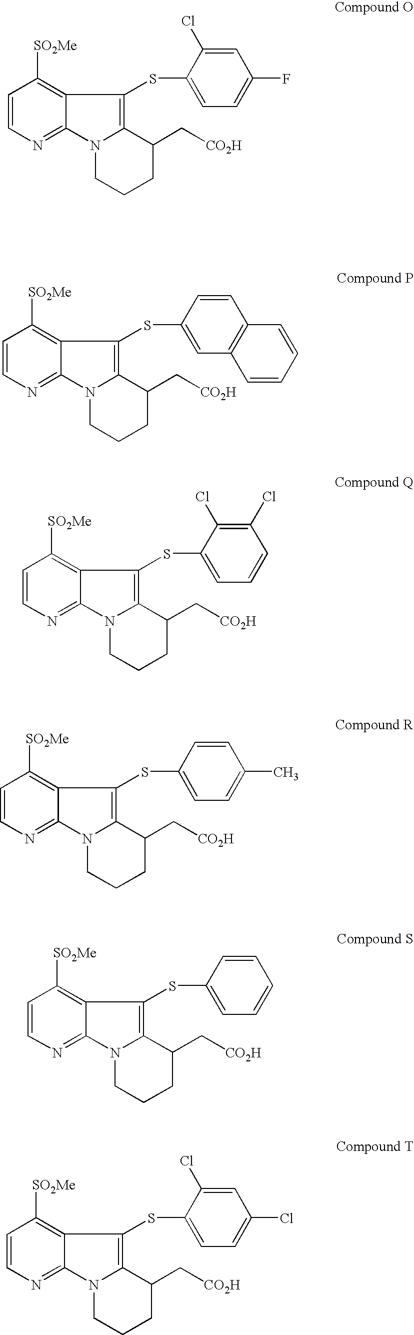 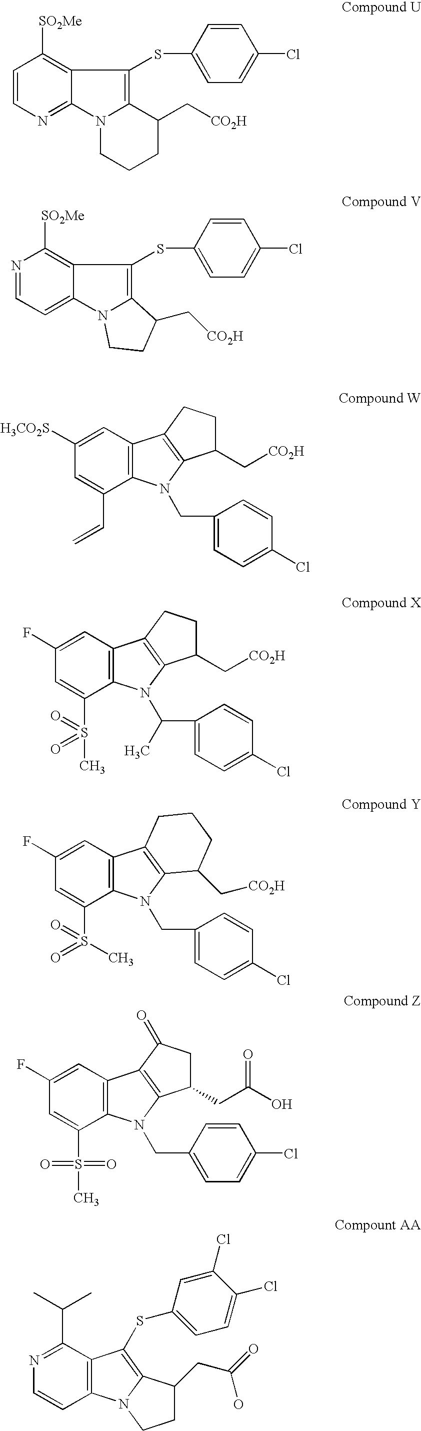 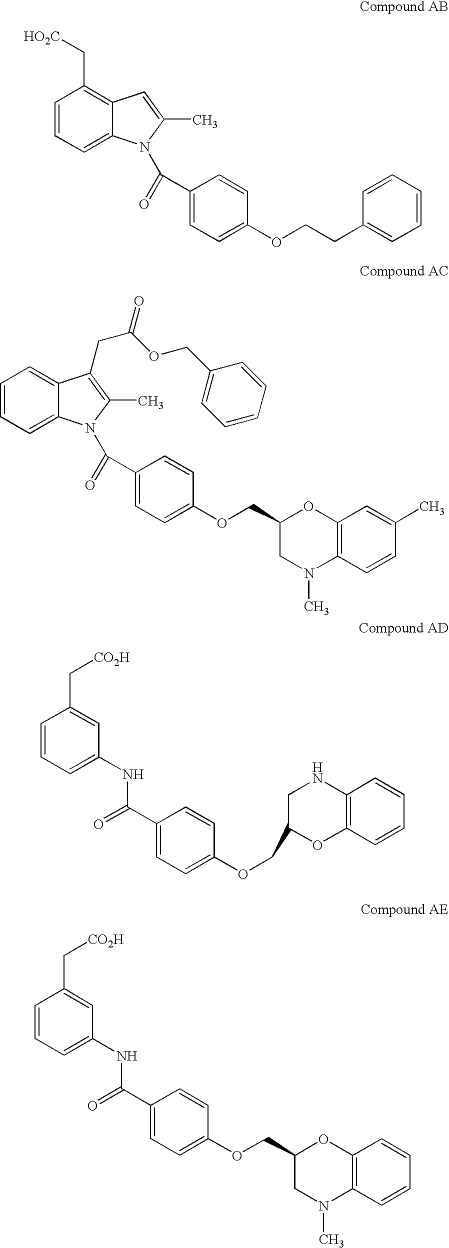 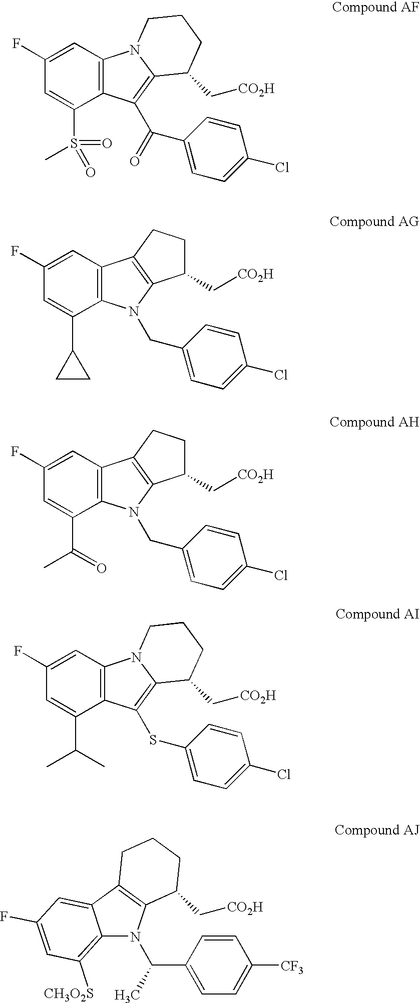
as well as the pharmaceutically acceptable salts and solvates thereof.

The compound of formula I or a pharmaceutically acceptable salt or solvate thereof and the DP antagonist can be administered together or sequentially in single or multiple daily doses, e.g., bid, tid or qid, without departing from the invention. If sustained release is desired, such as a sustained release product showing a release profile that extends beyond 24 hours, dosages may be administered every other day. However, single daily doses are preferred. Likewise, morning or evening dosages can be utilized.

Salts and Solvates

Salts and solvates of the compounds of formula I are also included in the present invention, and numerous pharmaceutically acceptable salts and solvates of nicotinic acid are useful in this regard. Alkali metal salts, in particular, sodium and potassium, form salts that are useful as described herein. Likewise alkaline earth metals, in particular, calcium and magnesium, form salts that are useful as described herein. Various salts of amines, such as ammonium and substituted ammonium compounds also form salts that are useful as described herein. Similarly, solvated forms of the compounds of formula I are useful within the present invention. Examples include the hemihydrate, mono-, di-, tri- and sesquihydrate.

The compounds of the invention also include esters that are pharmaceutically acceptable, as well as those that are metabolically labile. Metabolically labile esters include C₁₋₄ alkyl esters, preferably the ethyl ester. Many prodrug strategies are known to those skilled in the art. One such strategy involves engineered amino acid anhydrides possessing pendant nucleophiles, such as lysine, which can cyclize upon themselves, liberating the free acid. Similarly, acetone-ketal diesters, which can break down to acetone, an acid and the active acid, can be used.

The compounds used in the present invention can be administered via any conventional route of administration. The preferred route of administration is oral.

Pharmaceutical Compositions

The pharmaceutical compositions described herein are generally comprised of a compound of formula I or a pharmaceutically acceptable salt or solvate thereof, in combination with a pharmaceutically acceptable carrier.

Examples of suitable oral compositions include tablets, capsules, troches, lozenges, suspensions, dispersible powders or granules, emulsions, syrups and elixirs. Examples of carrier ingredients include diluents, binders, disintegrants, lubricants, sweeteners, flavors, colorants, preservatives, and the like. Examples of diluents include, for example, calcium carbonate, sodium carbonate, lactose, calcium phosphate and sodium phosphate. Examples of granulating and disintegrants include corn starch and alginic acid. Examples of binding agents include starch, gelatin and acacia. Examples of lubricants include magnesium stearate, calcium stearate, stearic acid and talc. The tablets may be uncoated or coated by known techniques. Such coatings may delay disintegration and thus, absorption in the gastrointestinal tract and thereby provide a sustained action over a longer period.

In one embodiment of the invention, a compound of formula I or a pharmaceutically acceptable salt or solvate thereof is combined with another therapeutic agent and the carrier to form a fixed combination product. This fixed combination product may be a tablet or capsule for oral use.

More particularly, in another embodiment of the invention, a compound of formula I or a pharmaceutically acceptable salt or solvate thereof (about 1 to about 1000 mg) and the second therapeutic agent (about 1 to about 500 mg) are combined with the pharmaceutically acceptable carrier, providing a tablet or capsule for oral use.

Sustained release over a longer period of time may be particularly important in the formulation. A time delay material such as glyceryl monostearate or glyceryl distearate may be employed. The dosage form may also be coated by the techniques described in the U.S. Pat. Nos. 4,256,108; 4,166,452 and 4,265,874 to form osmotic therapeutic tablets for controlled release.

Other controlled release technologies are also available and are included herein. Typical ingredients that are useful to slow the release of nicotinic acid in sustained release tablets include various cellulosic compounds, such as methylcellulose, ethylcellulose, propylcellulose, hydroxypropylcellulose, hydroxyethylcellulose, hydroxypropylmethylcellulose, microcrystalline cellulose, starch and the like. Various natural and synthetic materials are also of use in sustained release formulations. Examples include alginic acid and various alginates, polyvinyl pyrrolidone, tragacanth, locust bean gum, guar gum, gelatin, various long chain alcohols, such as cetyl alcohol and beeswax.

Optionally and of even more interest is a tablet as described above, comprised of a compound of formula I or a pharmaceutically acceptable salt or solvate thereof, and further containing an HMG Co-A reductase inhibitor, such as simvastatin or atorvastatin. This particular embodiment optionally contains the DP antagonist as well.

Typical release time frames for sustained release tablets in accordance with the present invention range from about 1 to as long as about 48 hours, preferably about 4 to about 24 hours, and more preferably about 8 to about 16 hours.

Hard gelatin capsules constitute another solid dosage form for oral use. Such capsules similarly include the active ingredients mixed with carrier materials as described above. Soft gelatin capsules include the active ingredients mixed with water-miscible solvents such as propylene glycol, PEG and ethanol, or an oil such as peanut oil, liquid paraffin or olive oil.

Aqueous suspensions are also contemplated as containing the active material in admixture with excipients suitable for the manufacture of aqueous suspensions. Such excipients include suspending agents, for example sodium carboxymethylcellulose, methylcellulose, hydroxypropylmethylcellulose, sodium alginate, polyvinylpyrrolidone, tragacanth and acacia; dispersing or wetting agents, e.g., lecithin; preservatives, e.g., ethyl, or n-propyl para-hydroxybenzoate, colorants, flavors, sweeteners and the like.

Dispersible powders and granules suitable for preparation of an aqueous suspension by the addition of water provide the active ingredients in admixture with a dispersing or wetting agent, suspending agent and one or more preservatives. Suitable dispersing or wetting agents and suspending agents are exemplified by those already mentioned above.

Syrups and elixirs may also be formulated.

More particularly, a pharmaceutical composition that is of interest is a sustained release tablet that is comprised of a compound of formula I or a pharmaceutically acceptable salt or solvate thereof, and a DP receptor antagonist that is selected from the group consisting of compounds A through AJ in combination with a pharmaceutically acceptable carrier.

Yet another pharmaceutical composition that is of more interest is comprised of a compound of formula I or a pharmaceutically acceptable salt or solvate thereof and a DP antagonist compound selected from the group consisting of compounds A, B, D, E, X, AA, AF, AG, AH, AI and AJ, in combination with a pharmaceutically acceptable carrier.

Yet another pharmaceutical composition that is of more particular interest relates to a sustained release tablet that is comprised of a compound of formula I or a pharmaceutically acceptable salt or solvate thereof, a DP receptor antagonist selected from the group consisting of compounds A, B, D, E, X, AA, AF, AG, AH, AI and AJ, and simvastatin or atorvastatin in combination with a pharmaceutically acceptable carrier.

The term “composition”, in addition to encompassing the pharmaceutical compositions described above, also encompasses any product which results, directly or indirectly, from the combination, complexation or aggregation of any two or more of the ingredients, active or excipient, or from dissociation of one or more of the ingredients, or from other types of reactions or interactions of one or more of the ingredients. Accordingly, the pharmaceutical composition of the present invention encompasses any composition made by admixing or otherwise combining the compounds, any additional active ingredient(s), and the pharmaceutically acceptable excipients.

Another aspect of the invention relates to the use of a compound of formula I or a pharmaceutically acceptable salt or solvate thereof and a DP antagonist in the manufacture of a medicament. This medicament has the uses described herein.

More particularly, another aspect of the invention relates to the use of a compound of formula I or a pharmaceutically acceptable salt or solvate thereof, a DP antagonist and an HMG Co-A reductase inhibitor, such as simvastatin, in the manufacture of a medicament. This medicament has the uses described herein.

Compounds of the present invention have anti-hyperlipidemic activity, causing reductions in LDL-C, triglycerides, apolipoprotein a and total cholesterol, and increases in HDL-C. Consequently, the compounds of the present invention are useful in treating dyslipidemias. The present invention thus relates to the treatment, prevention or reversal of atherosclerosis and the other diseases and conditions described herein, by administering a compound of formula I or a pharmaceutically acceptable salt or solvate in an amount that is effective for treating, preventing or reversing said condition. This is achieved in humans by administering a compound of formula I or a pharmaceutically acceptable salt or solvate thereof in an amount that is effective to treat or prevent said condition, while preventing, reducing or minimizing flushing effects in terms of frequency and/or severity.

One aspect of the invention that is of interest is a method of treating atherosclerosis in a human patient in need of such treatment comprising administering to the patient a compound of formula I or a pharmaceutically acceptable salt or solvate thereof in an amount that is effective for treating atherosclerosis in the absence of substantial flushing.

Another aspect of the invention that is of interest relates to a method of raising serum HDL levels in a human patient in need of such treatment, comprising administering to the patient a compound of formula I or a pharmaceutically acceptable salt or solvate thereof in an amount that is effective for raising serum HDL levels.

Another aspect of the invention that is of interest relates to a method of treating dyslipidemia in a human patient in need of such treatment comprising administering to the patient a compound of formula I or a pharmaceutically acceptable salt or solvate thereof in an amount that is effective for treating dyslipidemia.

Another aspect of the invention that is of interest relates to a method of reducing serum VLDL or LDL levels in a human patient in need of such treatment, comprising administering to the patient a compound of formula I or a pharmaceutically acceptable salt or solvate thereof in an amount that is effective for reducing serum VLDL or LDL levels in the patient in the absence of substantial flushing.

Another aspect of the invention that is of interest relates to a method of reducing serum triglyceride levels in a human patient in need of such treatment, comprising administering to the patient a compound of formula I or a pharmaceutically acceptable salt or solvate thereof in an amount that is effective for reducing serum triglyceride levels.

Another aspect of the invention that is of interest relates to a method of reducing serum Lp(a) levels in a human patient in need of such treatment, comprising administering to the patient a compound of formula I or a pharmaceutically acceptable salt or solvate thereof in an amount that is effective for reducing serum Lp(a) levels. As used herein Lp(a) refers to lipoprotein (a).

Another aspect of the invention that is of interest relates to a method of treating diabetes, and in particular, type 2 diabetes, in a human patient in need of such treatment comprising administering to the patient a compound of formula I or a pharmaceutically acceptable salt or solvate thereof in an amount that is effective for treating diabetes.

Another aspect of the invention that is of interest relates to a method of treating metabolic syndrome in a human patient in need of such treatment comprising administering to the patient a compound of formula I or a pharmaceutically acceptable salt or solvate thereof in an amount that is effective for treating metabolic syndrome.

Another aspect of the invention that is of particular interest relates to a method of treating atherosclerosis, dyslipidemias, diabetes, metabolic syndrome or a related condition in a human patient in need of such treatment, comprising administering to the patient a compound of formula I or a pharmaceutically acceptable salt or solvate thereof and a DP receptor antagonist, said combination being administered in an amount that is effective to treat atherosclerosis, dyslipidemia, diabetes or a related condition in the absence of substantial flushing.

Another aspect of the invention that is of particular interest relates to the methods described above wherein the DP receptor antagonist is selected from the group consisting of compounds A through AJ and the pharmaceutically acceptable salts and solvates thereof.

Methods of Synthesis for Compounds of Formula I

Compounds of formula I have been prepared by the following reaction schemes. It is understood that other synthetic routes to these structure classes are conceivable to one skilled in the art of organic synthesis. Therefore these reaction schemes should not be construed as limiting the scope of the invention. All substituents are as defined above unless indicated otherwise.

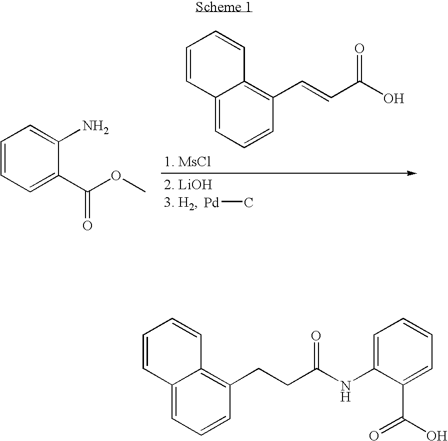

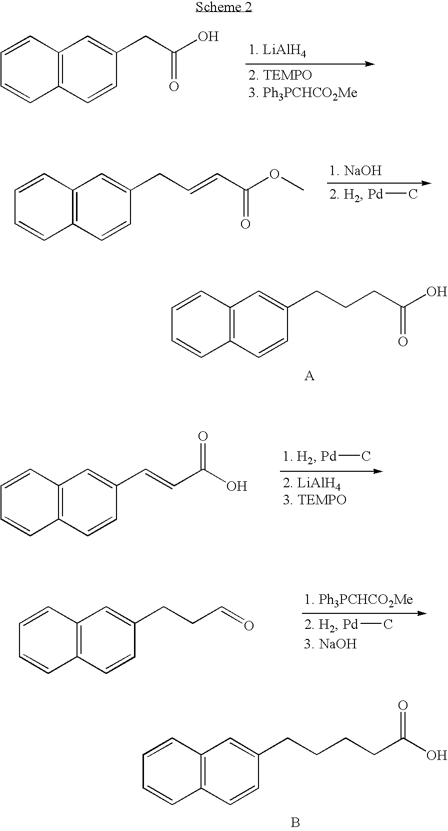

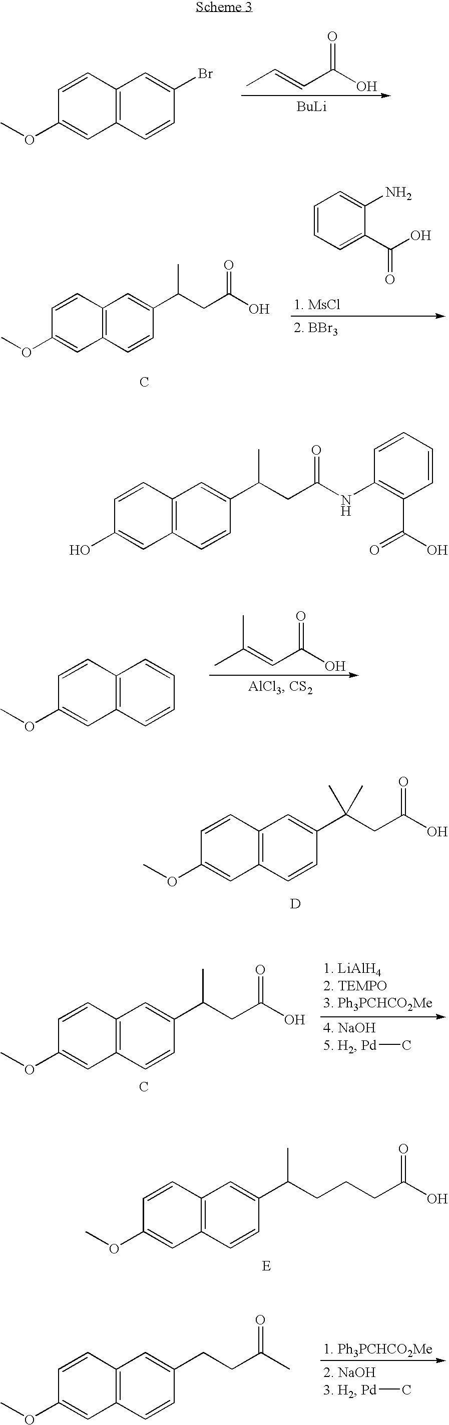

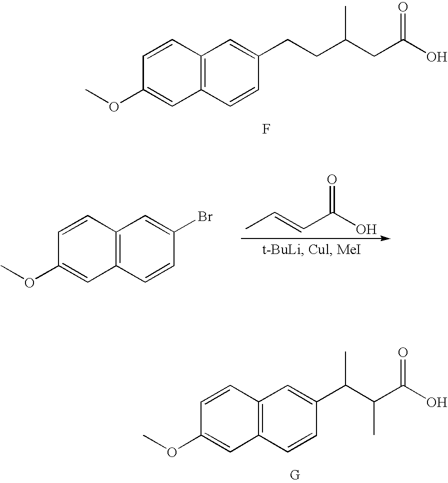

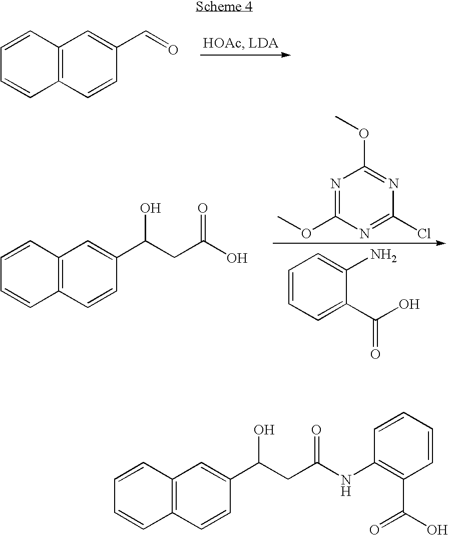

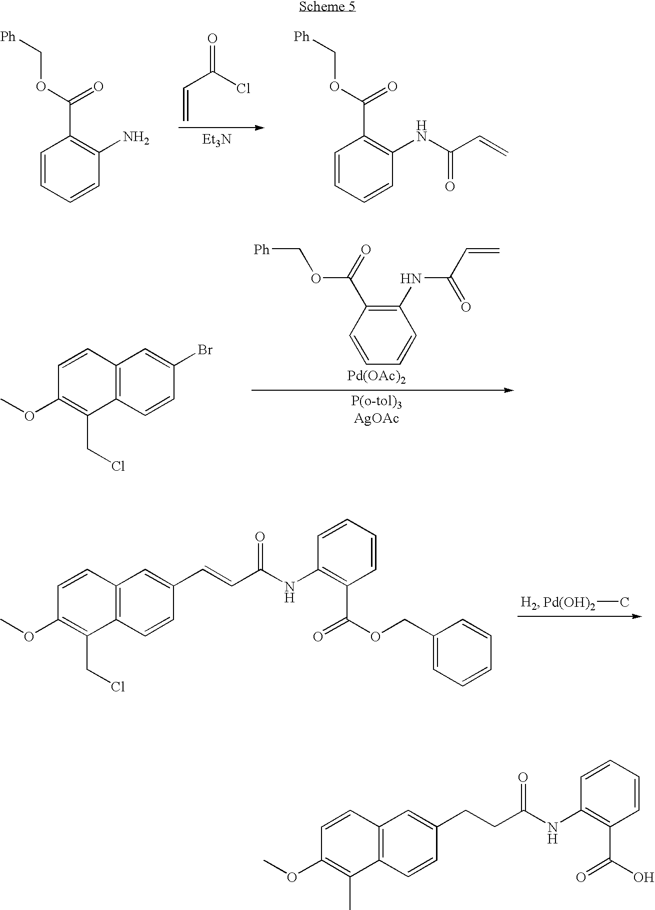

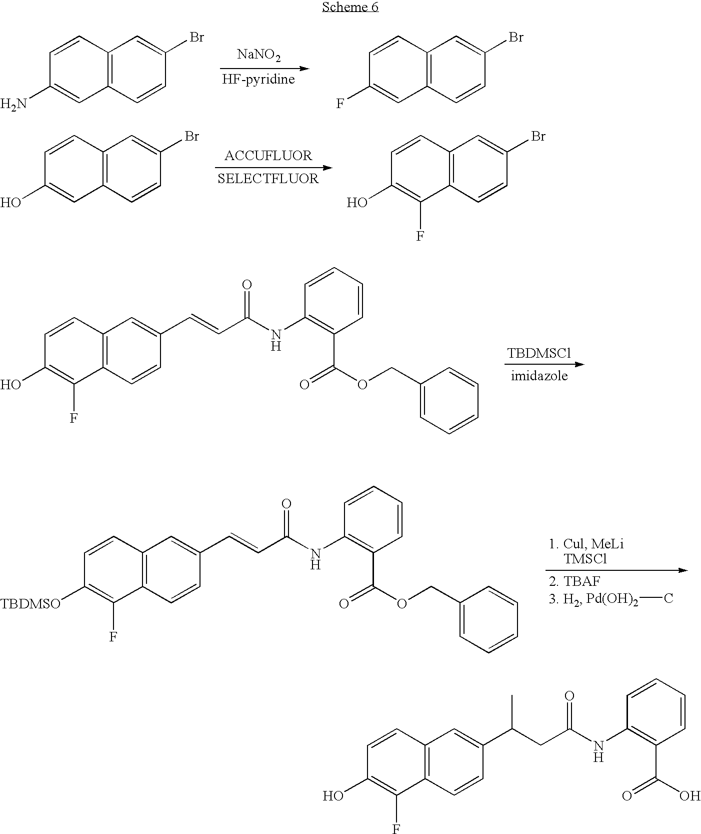

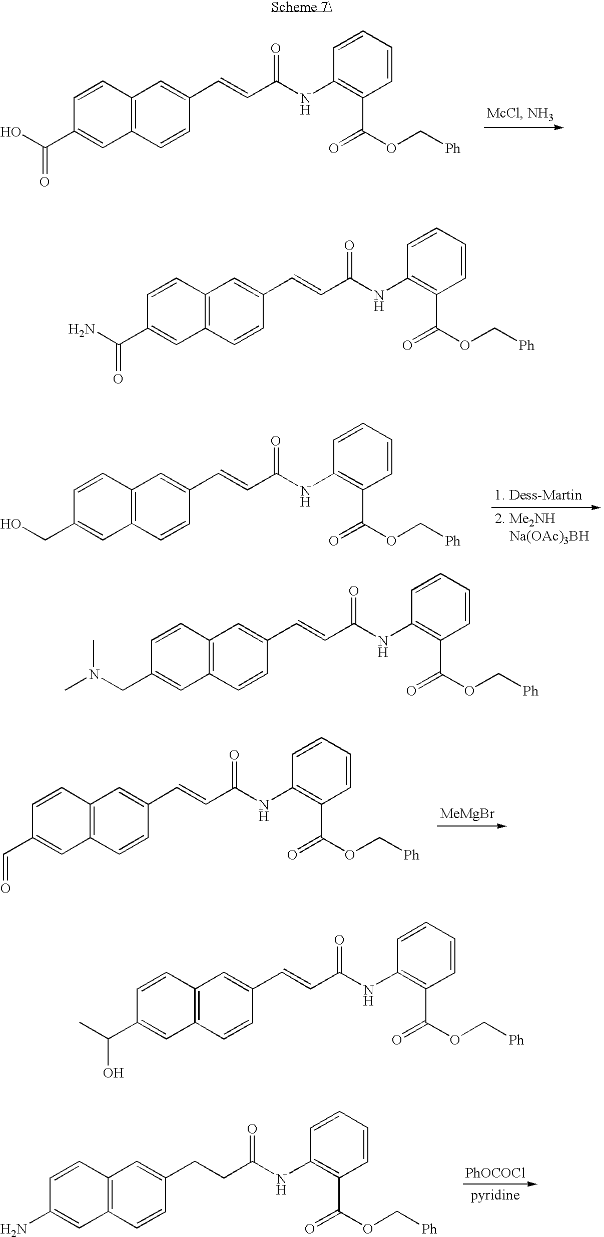

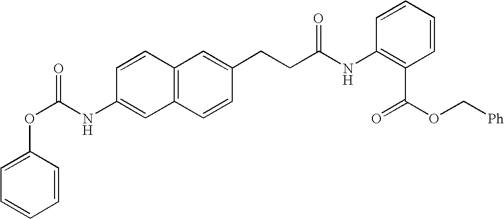

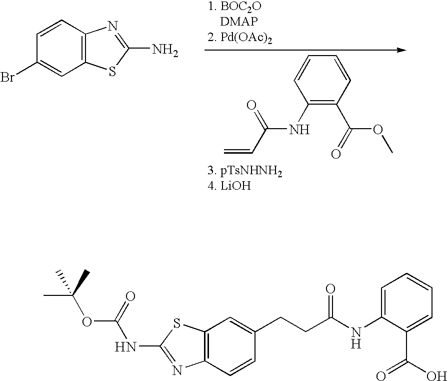

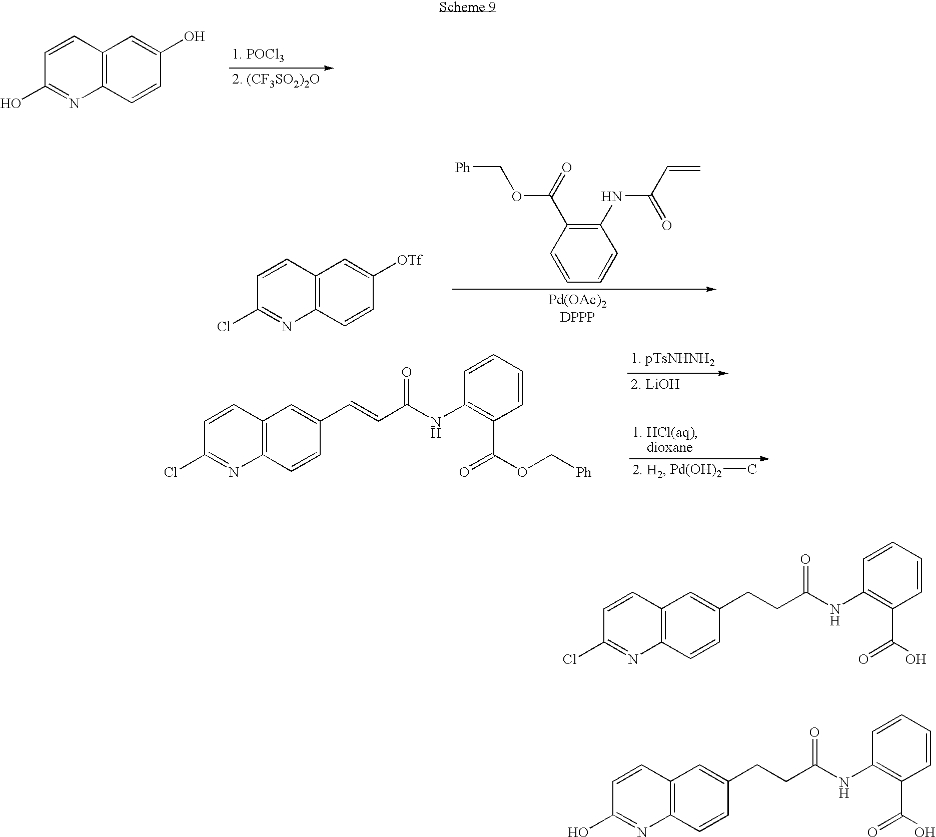

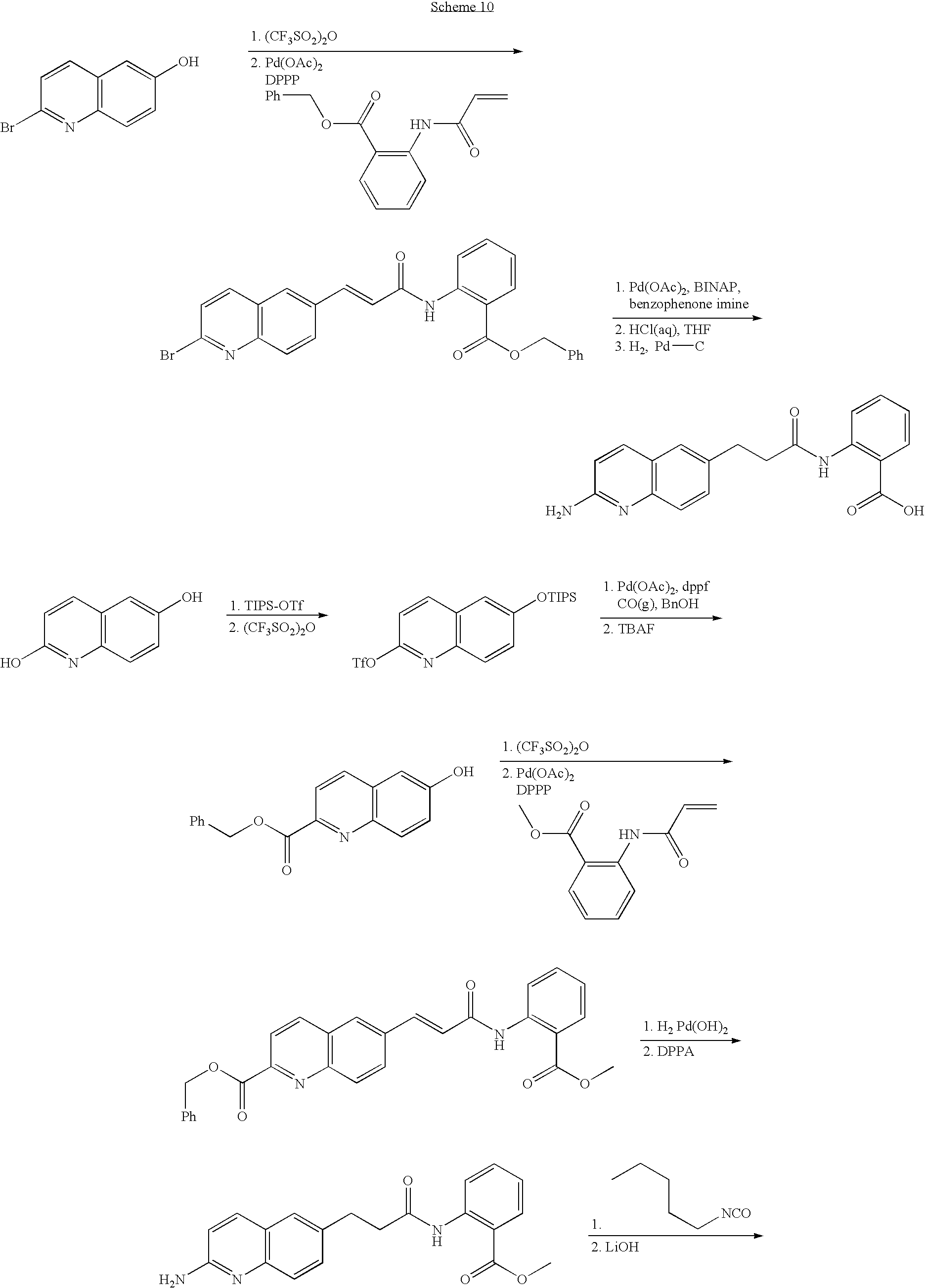

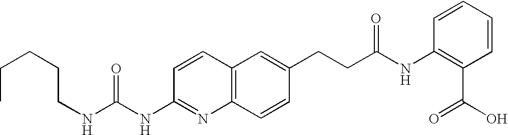

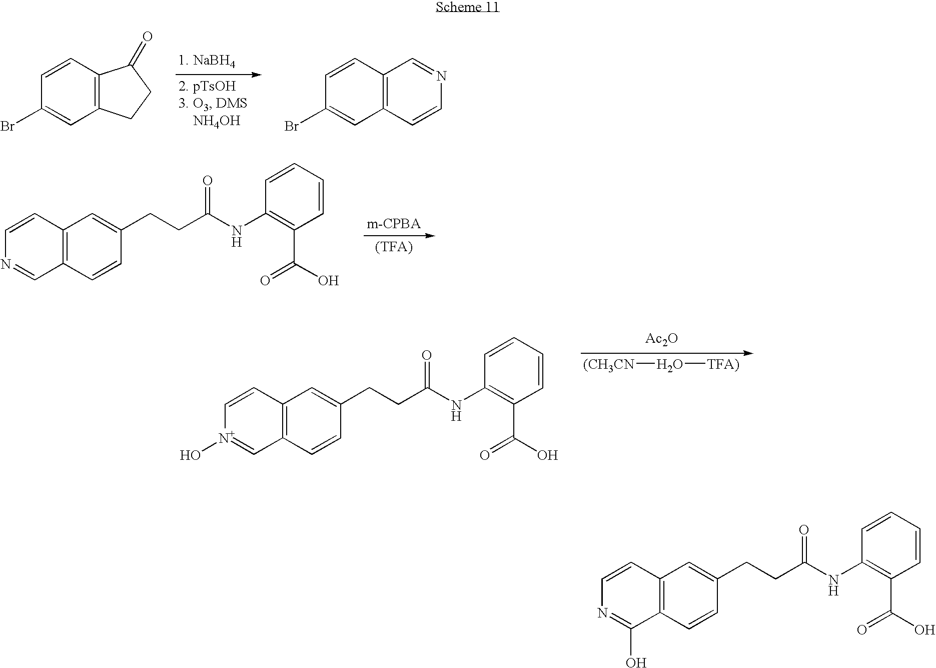

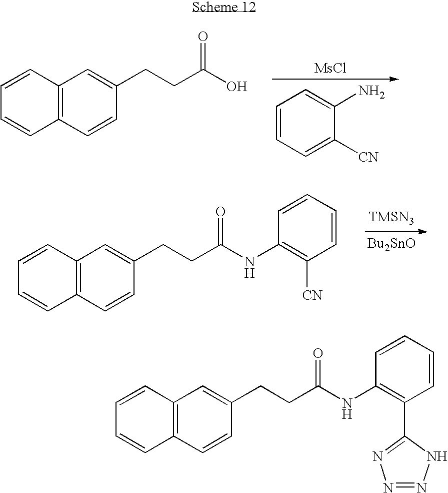

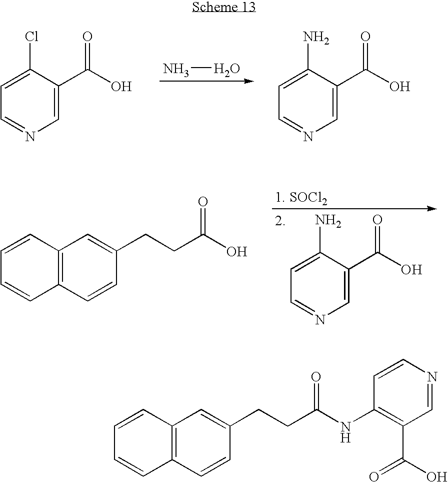

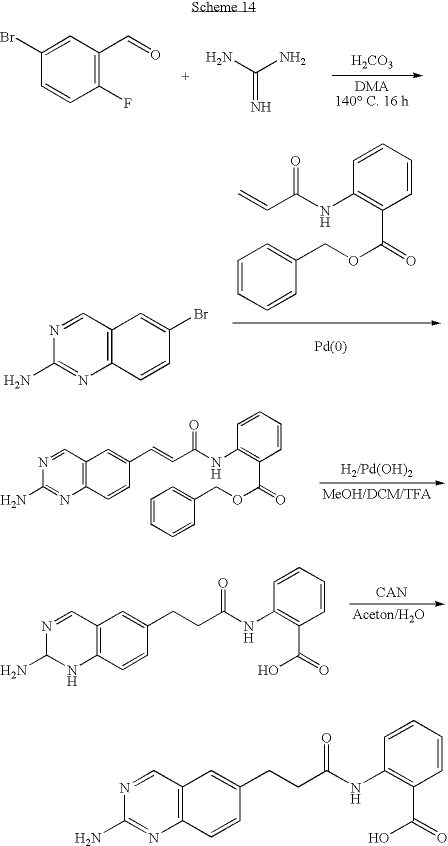

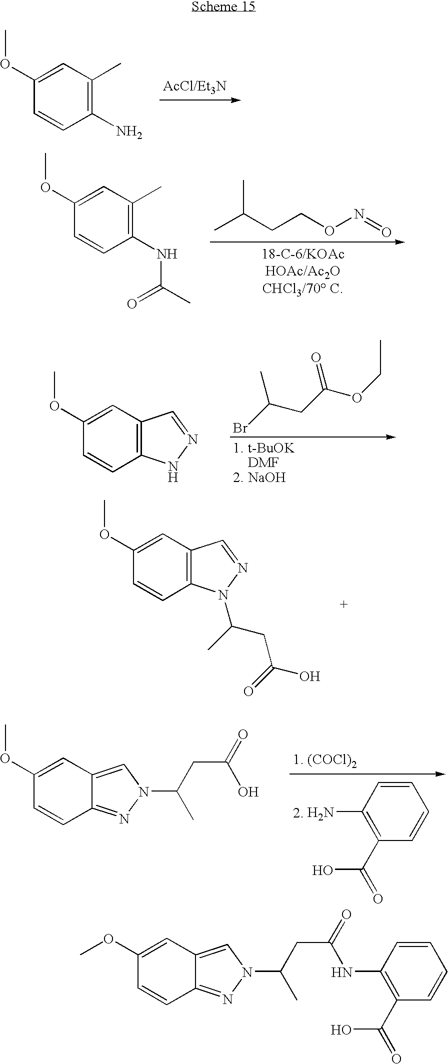

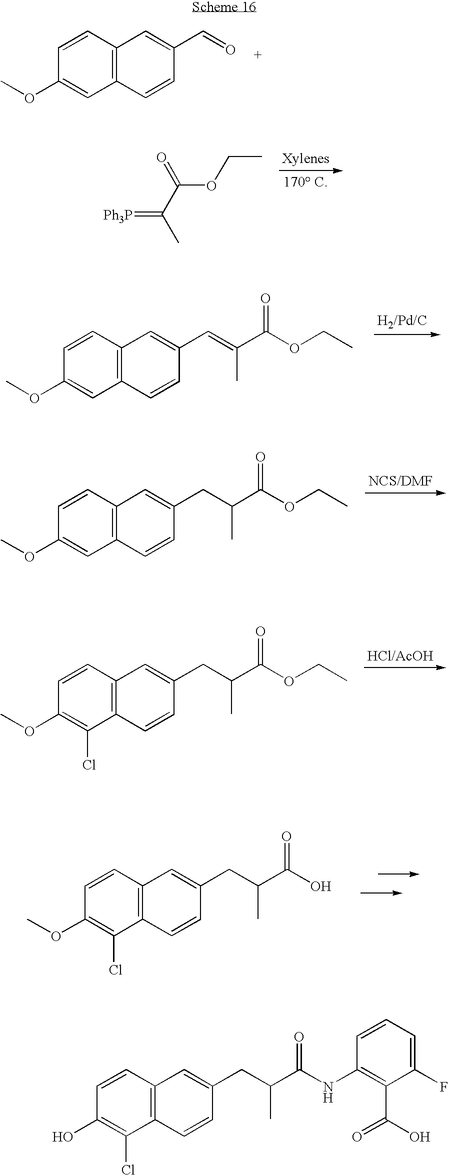

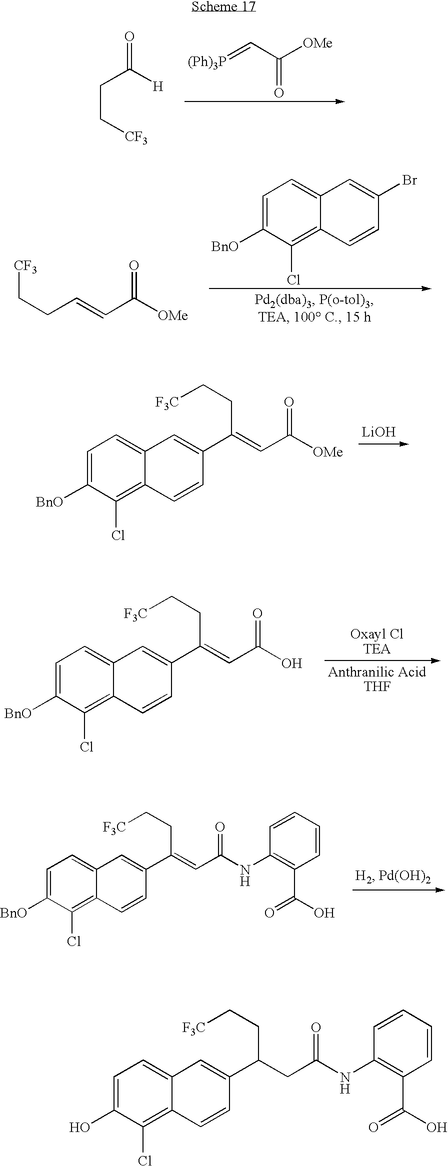

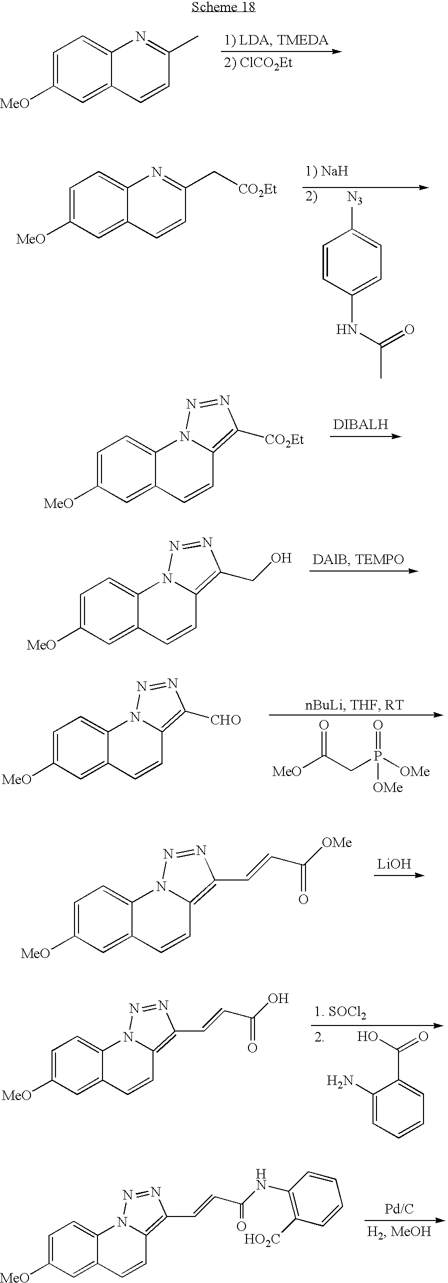

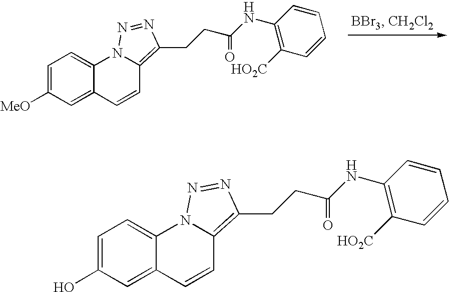

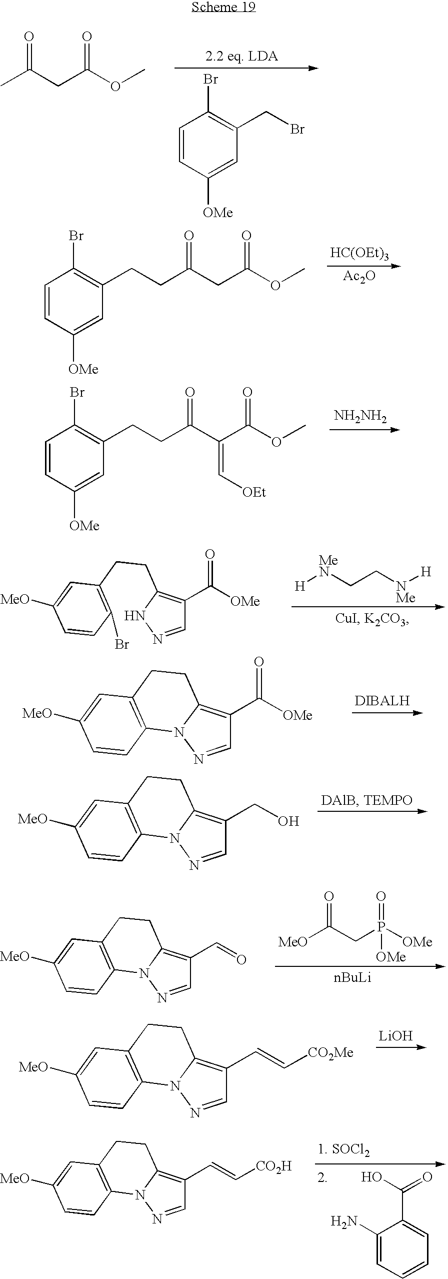

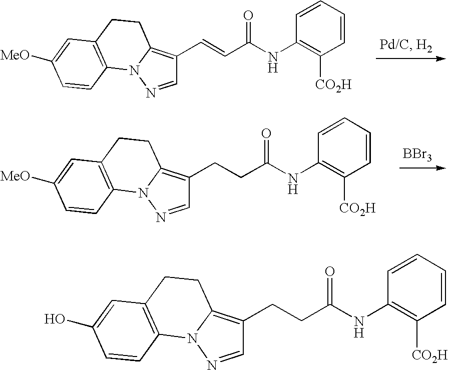

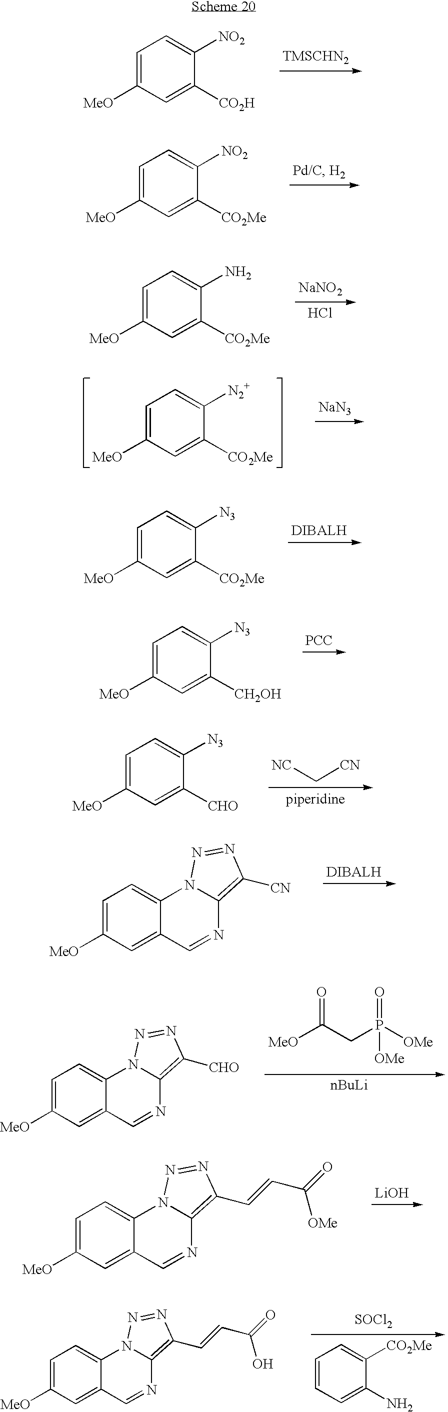

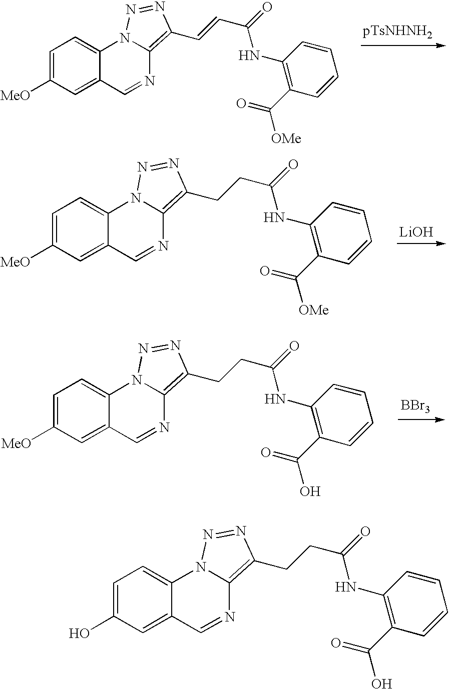

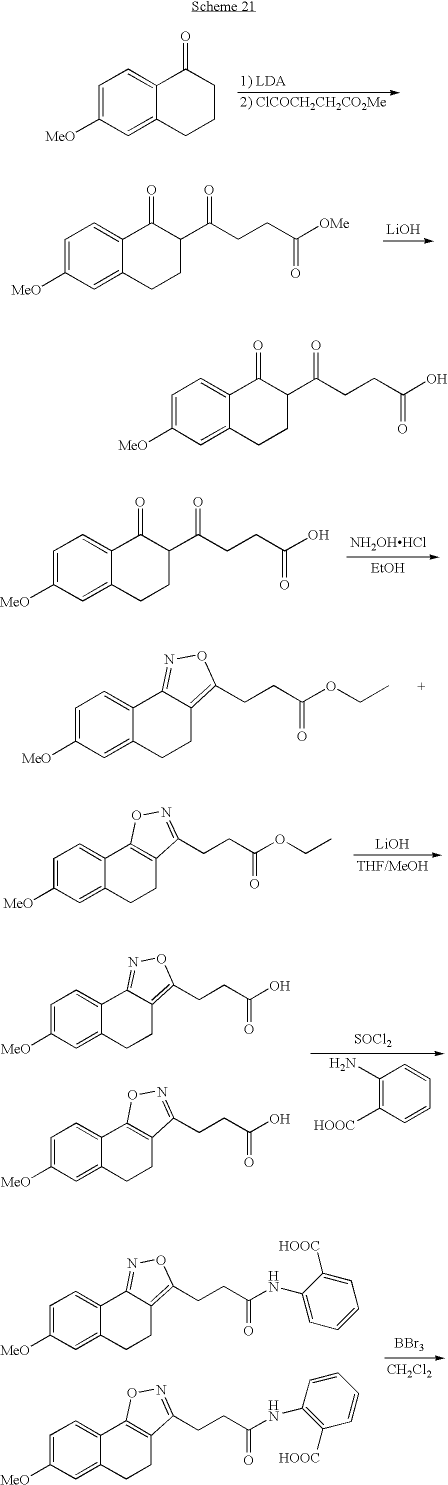

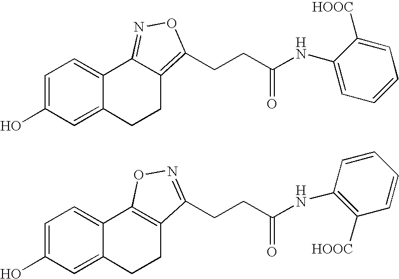

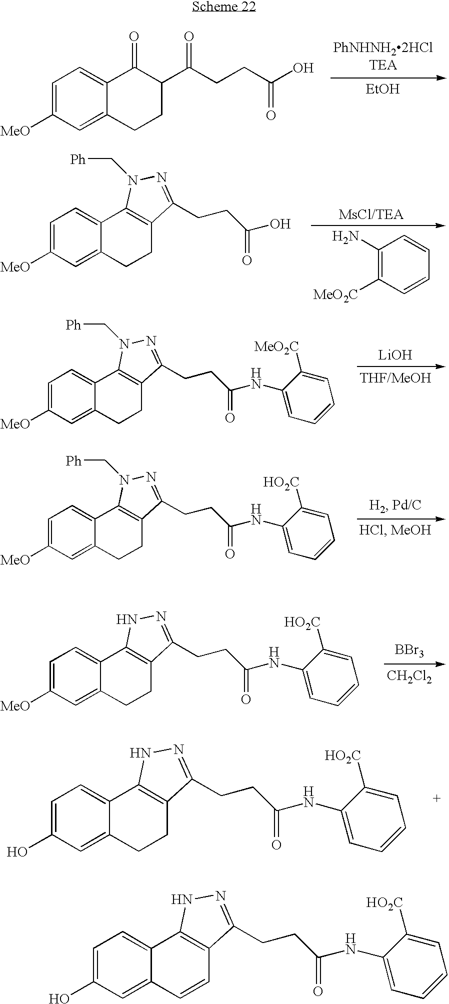

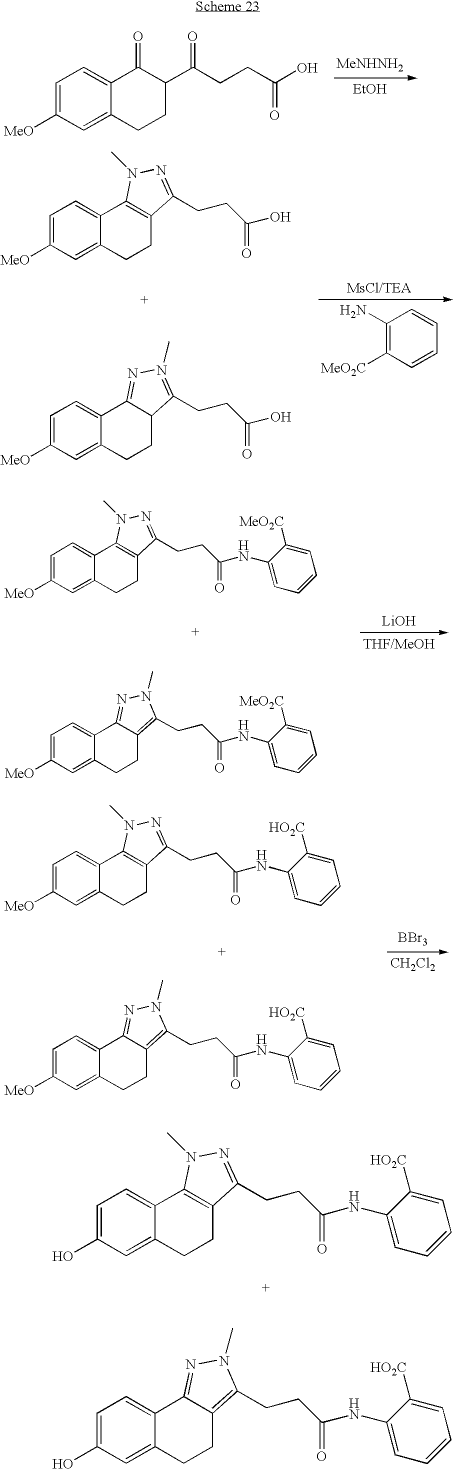

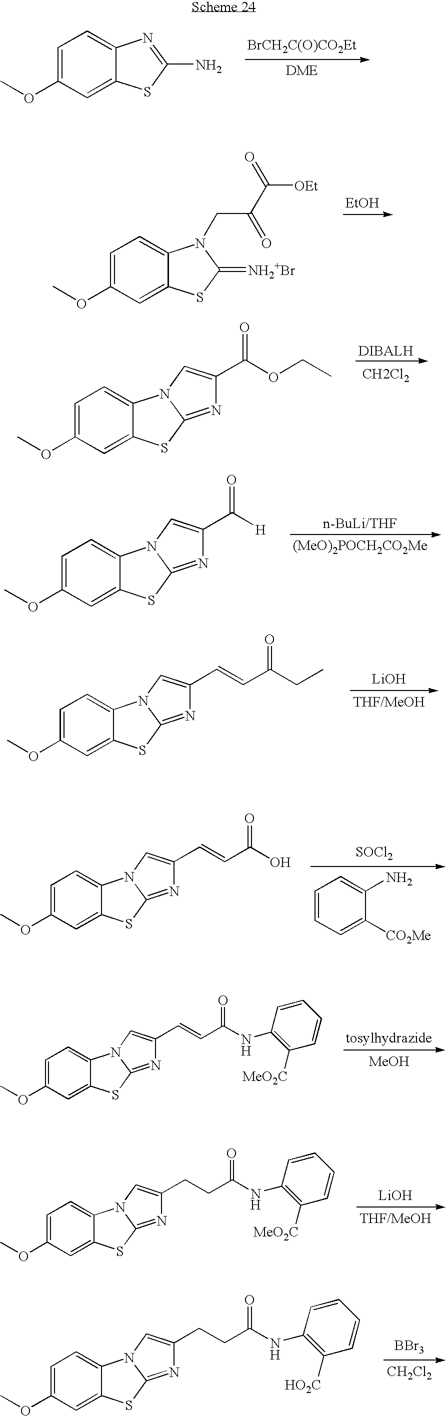

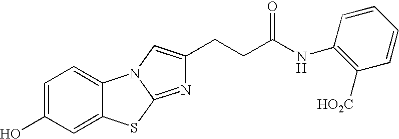

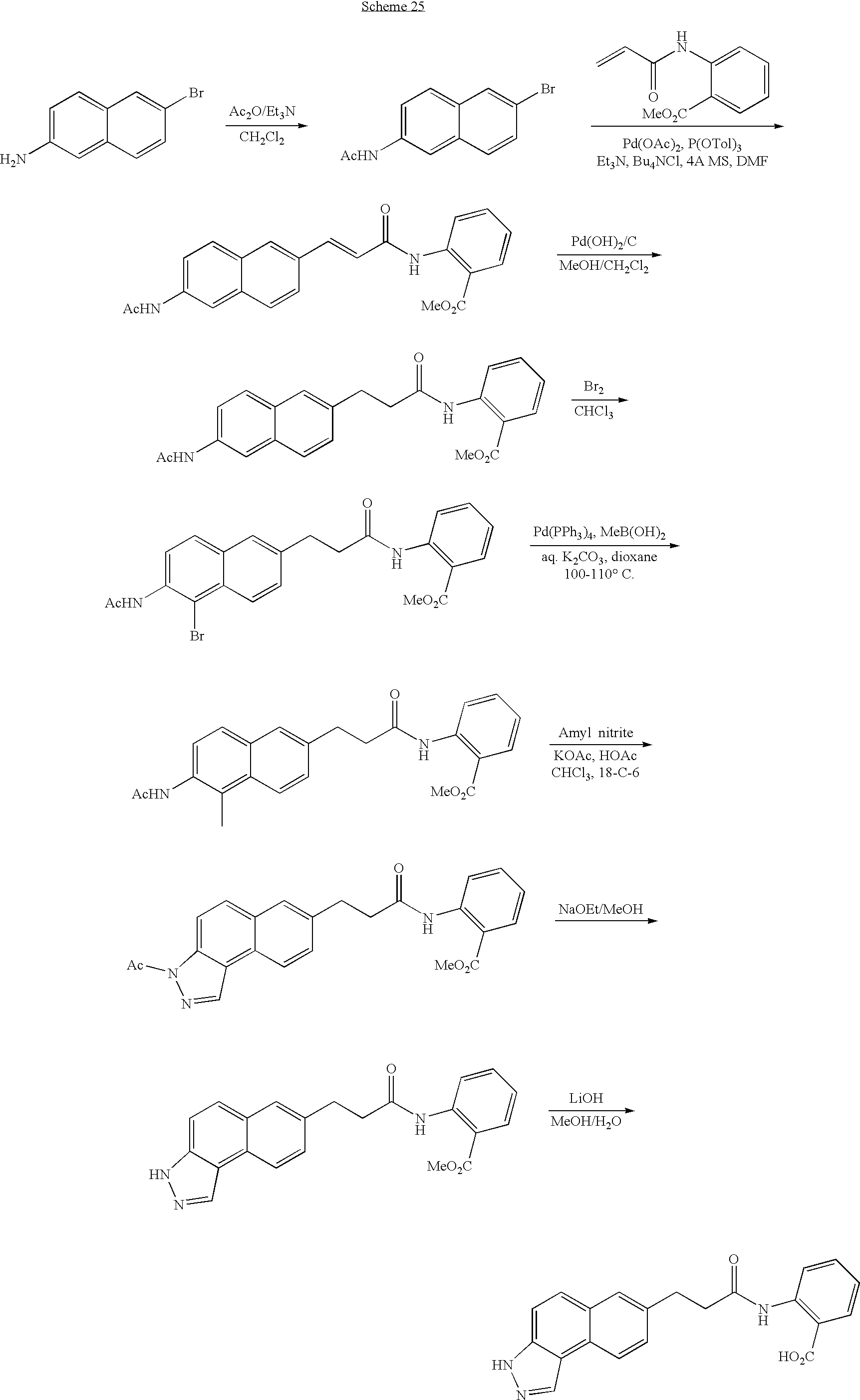

### REPRESENTATIVE EXAMPLES

The following examples are provided to more fully illustrate the present invention, and shall not be construed as limiting the scope in any manner. Unless stated otherwise:

(i) all operations were carried out at room or ambient temperature, that is, at a temperature in the range 18-25° C.;

(ii) evaporation of solvent was carried out using a rotary evaporator under reduced pressure (4.5-30 mmHg) with a bath temperature of up to 50° C.;

(iii) the course of reactions was followed by thin layer chromatography (TLC) and/or tandem high performance liquid chromatography (HPLC) followed by mass spectroscopy (MS), herein termed LCMS, and any reaction times are given for illustration only;

(iv) the structure of all final compounds was assured by at least one of the following techniques: MS or proton nuclear magnetic resonance (¹H NMR) spectrometry, and the purity was assured by at least one of the following techniques: TLC or HPLC;

(v) ¹H NMR spectra were recorded on either a Varian Unity or a Varian Inova instrument at 500 or 600 MHz using the indicated solvent; when line-listed, NMR data is in the form of delta values for major diagnostic protons, given in parts per million (ppm) relative to residual solvent peaks (multiplicity and number of hydrogens); conventional abbreviations used for signal shape are: s. singlet; d. doublet (apparent); t. triplet (apparent); m. multiplet; br. broad; etc.;

(vi) automated purification of compounds by preparative reverse phase HPLC was performed on a Gilson system using a YMC-Pack Pro C18 column (150×20 mm i.d.) eluting at 20 mL/min with 0-50% acetonitrile in water (0.1% TFA);

(vii) column chromatography was carried out on a glass silica gel column using Kieselgel 60, 0.063-0.200 mm (Merck), or a Biotage cartridge system;

(viii) MS data were recorded on a Waters Micromass unit, interfaced with a Hewlett-Packard (Agilent 1100) HPLC instrument, and operating on MassLynx/OpenLynx software; electrospray ionization was used with positive (ES+) or negative ion (ES−) detection; the method for LCMS ES+ was 1-2 mL/min, 10-95% B linear gradient over 5.5 min (B=0.05% TFA-acetonitrile, A=0.05% TFA-water), and the method for LCMS ES− was 1-2 mL/min, 10-95% B linear gradient over 5.5 min (B=0.1% formic acid−acetonitrile, A=0.1% formic acid−water), Waters XTerra C18-3.5 um-50×3.0 mmID and diode array detection;

(ix) the purification of compounds by preparative reverse phase HPLC(RPHPLC) was conducted on either a Waters Symmetry Prep C18-5 um-30×100 mmID, or a Waters Atlantis Prep dC18-5 um-20×100 mmID; 20 mL/min, 10-100% B linear gradient over 15 min (B=0.05% TFA-acetonitrile, A=0.05% TFA-water), and diode array detection;

(x) the purification of compounds by preparative thin layer chromatography (PTLC) was conducted on 20×20 cm glass prep plates coated with silica gel, commercially available from Analtech;

(xi) chemical symbols have their usual meanings; the following abbreviations have also been used v (volume), w (weight), b.p. (boiling point), m.p. (melting point), L (litre(s)), mL (millilitres), g (gram(s)), mg (milligrams(s)), mol (moles), mmol (millimoles), eq or equiv (equivalent(s)), IC50 (molar concentration which results in 50% of maximum possible inhibition), EC50 (molar concentration which results in 50% of maximum possible efficacy), uM (micromolar), nM (nanomolar);

(xii) the definitions of acronyms are as follows:

| | |
|---|---|
| THF is tetrahydrofuran | DMSO is dimethylsulfoxide |
| DMF is dimethylformamide | DMK is dimethylketone (acetone) |
| DMIDO is 1,3-dimethylimidazolidin-2-one | UV is ultraviolet |
| SELECTFLUOR is 1-(chloromethyl)-4- | ACCUFLUOR is 1-fluoro-4-hydroxy-1,4- |
| fluoro-1,4-diazoniabicyclo[2.2.2]octane | diazabicyclo[2.2.2]octane |
| bis(tetrafluoroborate) | bis(tetrafluoroborate) |
| EDCI is 1-ethyl-3-(3- | DPPP is 1,3-bis(diphenylphosphino)propane |
| dimethylaminopropyl)carbodiimide | |
| hydrochloride | |
| TEMPO is 2,2,6,6-tetramethyl-1- | BINAP is 2,2′-bis(diphenylphosphino)-1,1′- |
| piperidinyloxy, free radical | binaphthyl |
| DPPF is 1,1′- | DMAP is 4-(N,N-dimethylamino)pyridine |
| bis(diphenylphosphino)ferrocene | |
| RT is room temperature | DMA is dimethylacetamide |
| DIBALH is diisobutylaluminum hydride | DME is 1,2-dimethoxyethane |
| (ee) is enantiomeric excess | |

### Example 1

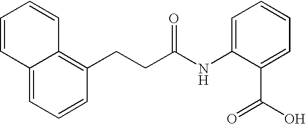

Commercially available 3(1-naphthyl)acrylic acid (250 mg, 1.26 mmol) was dissolved in 6 mL of anhydrous methylene chloride under nitrogen atmosphere, treated with triethylamine (525 uL, 3.78 mmol) and then methanesulfonyl chloride (310 uL, 3.78 mmol). The reaction mixture was then treated with methyl anthranilate (163 uL, 1.26 mmol), aged and monitored hourly by LCMS. The reaction mixture was partitioned between saturated aqueous NaHCO₃ and methylene chloride, the organic phase was separated and dried over anhydrous sodium sulfate, and then evaporated under reduced pressure. The crude product was saponified directly with excess aqueous 1N LiOH in (3:1:1) THF-MeOH—H₂O. The reaction mixture was concentrated to a minimal volume, co-dissolved with DMSO, and purified directly via preparative RPHPLC. A portion of this enoic acid product (8 mg, 0.025 mmol) was then dissolved in ethyl acetate (2 mL), treated with catalytic palladium on carbon, and hydrogenated at 1 atmosphere with a hydrogen-filled balloon. The reaction mixture was filtered over celite and concentrated in vacuo. The residue was purified via preparative RPHPLC to give the desired product.

¹H NMR (acetone-d₆, 500 MHz) δ 11.3 (s, 1H), 8.8 (d, 1H), 8.2 (d, 1H), 8.1 (d, 1H), 7.9 (d, 1H), 7.8 (d, 1H), 7.6 (m, 2H) 7.5 (m, 2H), 7.4 (m, 1H), 7.2 (t, 1H), 3.6 (t, 2H), 2.9 (t, 2H); LCMS m/z 320 (M⁺+1).

### Example 2

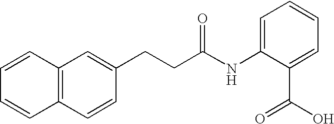

EXAMPLE 2 was prepared in a similar manner as in EXAMPLE 1 and illustrated in Scheme 1 from the commercially available 3(2-naphthyl)acrylic acid: ¹H NMR (DMSO-d₆, 500 MHz) δ 11.2 (s, 1H), 8.5 (d, 1H), 8.0 (d, 1H), 7.9 (m, 3H), 7.8 (s, 1H), 7.6 (t, 1H), 7.5 (m, 3H), 7.1 (t, 1H), 3.1 (t, 2H), 2.8 (t, 2H); LCMS m/z 320 (M⁺+1), 342 (M⁺+Na).

### Example 3

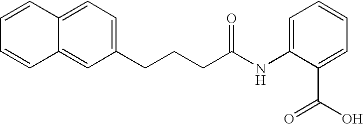

Commercially available 2-naphthylacetic acid (3 g, 16.1 mmol) in 8 mL of anhydrous diethyl ether was added dropwise to a solution of lithium aluminum hydride (1.2 g, 32.2 mmol) in 8 mL of anhydrous diethyl ether under nitrogen atmosphere. The reaction mixture was aged for 2 h, quenched with aqueous Rochelle salt, stirred for an additional 2 h, partitioned between saturated aqueous NaHCO₃ and diethyl ether, the organic phase was separated and dried over anhydrous sodium sulfate, and then evaporated under reduced pressure to provide the crude alcohol product (1.4 g). This alcohol (1.0 g, 5.81 mmol) was oxidized directly with iodobenzene diacetate (2.1 g, 6.5 mmol) and catalytic TEMPO (10%) in methylene chloride solvent (20 mL). The reaction mixture was quenched with aqueous sodium thiosulfate, partitioned with methylene chloride, the organic phase washed with aqueous NaHCO₃, and the organic phase concentrated in vacuo to provide the clean aldehyde product. This crude aldehyde intermediate (500 mg, 2.9 mmol) was combined with methyl (triphenylphosphoranylidene) acetate (1.47 g, 4.4 mmol) in toluene (10 mL), and the reaction mixture heated at reflux for 4 h. The mixture was concentrated in vacuo to a residue which was purified by flash column chromatography (SiO₂, EtOAc/hexanes) to give the desired methyl enoate. This intermediate was then dissolved in tetrahydrofuran (10 mL), treated with aqueous 1N NaOH (5 mL), refluxed for 2 h, the mixture cooled, acidified and extracted with ethyl acetate. The organic phase was concentrated in vacuo to provide the enoic acid, which was treated with catalytic palladium on carbon in methanol (20 mL), and hydrogenated at 1 atmosphere with a hydrogen-filled balloon for 12 h. The reaction mixture was filtered over celite and concentrated in vacuo to provide the clean crude acid defined as Compound A in Scheme 2. Compound A (50 mg, 0.234 mmol) was converted into EXAMPLE 3 in a similar manner as in EXAMPLE 1 and illustrated in Scheme 1 using anthranilic acid directly in the amide coupling reaction. The product was purified via preparative RPHPLC and then recrystallization (diethyl ether/hexane) to give the desired product. ¹H NMR (CDCl₃, 500 MHz) δ 10.93 (s, 1H), 8.79 (d, 1H), 8.11 (d, 1H), 7.80 (m, 3H), 7.68 (s, 1H), 7.61 (t, 1H), 7.40 (m, 2H), 7.38 (d, 1H), 7.12 (t, 1H), 2.92 (t, 2H), 2.53 (t, 2H), 2.22 (m, 2H); LCMS m/z 332 (M⁺−1).

### Example 4

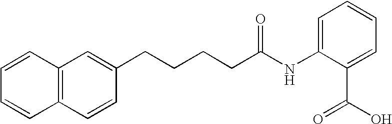

Commercially available 3(2-naphthyl)acrylic acid (5 g) in 50 mL of (1:1) methanol-methylene chloride was treated with catalytic palladium on carbon, and hydrogenated at 1 atmosphere with a hydrogen-filled balloon for 12 h. The reaction mixture was filtered over celite and concentrated in vacuo to provide the clean crude acid. This intermediate (1 g, 5 mmol) in diethyl ether (100 mL) was added dropwise to a solution of lithium aluminum hydride (380 mg, 10 mmol) in 100 mL of anhydrous diethyl ether under nitrogen atmosphere. The reaction mixture was aged for 12 h, quenched with aqueous Rochelle salt, stirred for an additional 2 h, partitioned between saturated aqueous NaHCO₃ and diethyl ether, the organic phase was separated and dried over anhydrous sodium sulfate, and then evaporated under reduced pressure to provide the crude alcohol product. This alcohol (1.0 g, 5.4 mmol) was oxidized directly with iodobenzene diacetate (1.7 g, 5.9 mmol) and catalytic TEMPO (10%) in methylene chloride solvent (30 mL). After 2 h, the reaction mixture was quenched with aqueous sodium thiosulfate, partitioned with methylene chloride, the organic phase washed with aqueous NaHCO₃, and the organic phase concentrated in vacuo to provide the clean aldehyde product as an oil. This crude aldehyde intermediate (240 mg, 1.3 mmol) was combined with methyl (triphenylphosphoranylidene) acetate (650 mg, 1.94 mmol) in toluene (5 mL), and the reaction mixture heated at reflux for 2 h. The mixture was concentrated in vacuo to a residue which was purified by flash column chromatography (SiO₂, EtOAc/hexanes) to give the desired methyl enoate. This intermediate was then treated with catalytic palladium on carbon in methanol (10 mL), and hydrogenated at 1 atmosphere with a hydrogen-filled balloon for 4 h. The reaction mixture was filtered over celite and concentrated in vacuo to provide the clean crude ester which was dissolved in (3:1:1) THF-MeOH—H₂O (10 mL), treated with aqueous 1N NaOH (2.6 mL), aged for 6 h, the mixture acidified and extracted with diethyl ether. The organic phase was concentrated in vacuo to provide the clean acid, which is defined as Compound B in Scheme 2. Compound B (50 mg, 0.22 mmol) was converted into EXAMPLE 4 in a manner similar to EXAMPLE 1 and illustrated in Scheme 1 using anthranilic acid directly in the amide coupling reaction. The product was purified via preparative RPHPLC to give the desired product. ¹H NMR (CDCl₃, 500 MHz) δ 8.78 (d, 1H), 8.12 (d, 1H), 7.81 (d, 1H), 7.77 (d, 2H), 7.65 (s, 1H), 7.62 (t, 1H), 7.43 (m, 2H), 7.35 (d, 1H), 7.14 (t, 1H), 2.87 (t, 2H), 2.53 (t, 2H), 1.86 (m, 4H); LCMS m/z 346 (M⁺−1).

### Example 5

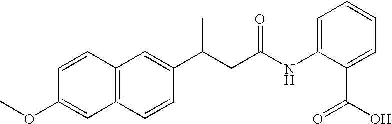

Commercially available 2-bromo-6-methoxynaphthalene (2.9 g, 12.2 mmol) in anhydrous tetrahydrofuran (20 mL) was chilled to −78° C. under nitrogen, and treated dropwise with a solution of n-butyllithium (1.6 M, 7.6 mL, 12.2 mmol). The reaction mixture was aged for 10 min, and then treated with a solution of 2-butenoic acid (500 mg, 5.8 mmol) in 30 mL of anhydrous tetrahydrofuran under nitrogen atmosphere. The reaction mixture was aged for 1 h at −78° C., quenched with water, partitioned with ethyl acetate, the aqueous phase acidified with 2N HCl to pH 2, washed with ethyl acetate, the organic phase was separated and dried over anhydrous sodium sulfate, and then evaporated under reduced pressure to provide the crude acid product which is defined as Compound C in Scheme 3. Compound C (120 mg, 0.49 mmol) was converted into EXAMPLE 5 in a manner similar to EXAMPLE 1 and illustrated in Scheme 1 using anthranilic acid directly in the amide coupling reaction. The product was purified via preparative RPHPLC to give the desired product. ¹H NMR (CDCl₃, 500 MHz) δ 10.88 (s, 1H), 8.75 (d, 1H), 8.11 (d, 1H), 7.71 (d, 2H), 7.68 (s, 1H), 7.60 (t, 1H), 7.41 (d, 1H), 7.14 (m, 3H), 3.92 (s, 3H), 3.60 (m, 1H), 2.87 (m, 1H), 2.76 (m, 1H), 1.49 (d, 3H); LCMS m/z 362 (M⁺−1).

### Example 6

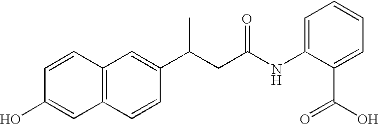

EXAMPLE 5 (27 mg, 0.074 mmol) in anhydrous methylene chloride (3 mL) was chilled to −78° C. under nitrogen, and treated with a solution of boron tribromide (1M, 0.45 mL, 0.45 mmol). The reaction mixture was warmed to room temperature, aged for 3 h, and then partitioned between methylene chloride and water, the organic phase was separated and dried over anhydrous sodium sulfate, and then evaporated under reduced pressure. The product was purified via preparative RPHPLC to give the desired product. ¹H NMR (CDCl₃, 500 MHz) δ 10.88 (s, 1H), 8.74 (d, 1H), 8.08 (d, 1H), 7.70 (d, 1H), 7.67 (s, 1H), 7.65 (d, 2H), 7.61 (d, 1H), 7.44 (d 1H), 7.13 (m, 2H), 7.08 (d, 1H), 3.59 (m, 1H), 2.85 (m, 1H), 2.76 (m, 1H), 1.48 (d, 3H); LCMS m/z 348 (M⁺−1).

### Chiral Resolution of Compound C as its Methyl Ester

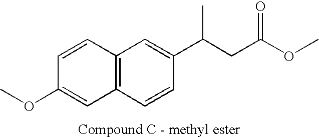

Compound C in Scheme 3 can also be generated as its methyl ester by a Heck coupling of commercially available 2-bromo-6-methoxynaphthalene with methyl 2-butenoate in the presence of catalytic palladium acetate, P(O-tol)₃, and triethylamine at 100° C. for 5 h. Following standard hydrogenation conditions (Pd—C in methanol) to reduce the resultant olefin, the racemic methyl ester of Compound C was resolved into its enantiomers: Preparative Chiralcel OJ column; isocratic elution with 35% isopropanol-heptane; 9 mL/min; UV=229 nm; retention times of 26.83 minutes (99.9% ee) and 31.50 minutes (92% ee). Upon demethylation of the methyl esters with potassium trimethylsilanolate in THF, the subsequent single enantiomers of Compound C in Scheme 3 were converted into single enantiomers of EXAMPLES 5 and 6 under conditions described above.

### Example 7

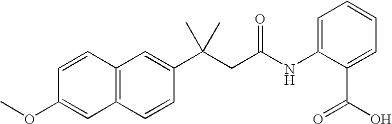

Commercially available 2-methoxynaphthalene (6.3 g, 36.6 mmol) was combined with AlCl₃ (2.7 g, 20 mmol) in CS₂ (30 mL) at 0° C., and the mixture treated with a solution of 3-methylcrotonic acid (2 g, 20 mmol) in CS₂ (15 mL) over 40 min. The mixture was treated again with AlCl₃ (2.7 g, 20 mmol) and a solution of 3-methylcrotonic acid (2 g, 20 mmol) in CS₂ (15 mL) at 0° C. The mixture was aged for 2 h, warmed to room temperature, aged further for 6 h, and then quenched with aqueous 4% NaOH, the aqueous phase acidified with cold concentrated HCl, extracted with diethyl ether, the organic phase was separated and dried over anhydrous sodium sulfate, and then evaporated under reduced pressure to provide the crude acid product which is defined as Compound D in Scheme 3. Compound D (150 mg, 0.58 mmol) was converted into EXAMPLE 7 in a manner similar to EXAMPLE 1 and illustrated in Scheme 1 using anthranilic acid directly in the amide coupling reaction. The product was purified via preparative RPHPLC to give the desired product. ¹H NMR (CD₃OD, 500 MHz) δ 8.44 (d, 1H), 7.99 (d, 1H), 7.76 (s, 1H), 7.71 (t, 2H), 7.59 (d 1H), 7.48 (t, 1H), 7.16 (m, 1H), 7.07 (m, 2H), 3.90 (s, 3H), 2.79 (s, 2H), 1.62 (s, 6H); LCMS m/z 376 (M⁺−1).

### Example 8

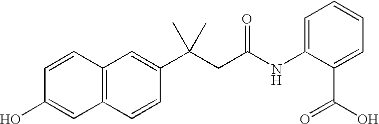

EXAMPLE 8 was prepared from EXAMPLE 7 (33.6 mg, 0.09 mmol) in a manner similar to EXAMPLE 6 and illustrated in Scheme 3 using boron tribromide. The product was purified via preparative RPHPLC to give the desired product. ¹H NMR (CDCl₃, 500 MHz) δ 10.94 (s, 1H), 8.79 (d, 1H), 8.00 (d, 1H), 7.73 (s, 1H), 7.66 (d, 1H), 7.59 (d 1H), 7.54 (d, 1H), 7.48 (t, 1H), 7.08 (t, 1H), 7.02 (m, 2H), 2.78 (s, 2H), 1.61 (s, 6H); LCMS m/z 362 (M⁺−1).

### Example 9

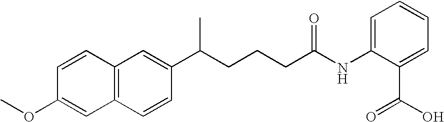

Compound C from EXAMPLE 5 (250 mg, 1.0 mmol) in diethyl ether (15 mL) was added dropwise to a solution of lithium aluminum hydride (76 mg, 2.0 mmol) in 15 mL of anhydrous diethyl ether under nitrogen atmosphere. The reaction mixture was aged, quenched with aqueous Rochelle salt, stirred for an additional 2 h, partitioned between saturated aqueous NaHCO₃ and diethyl ether, the organic phase was separated and dried over anhydrous sodium sulfate, and then evaporated under reduced pressure to provide the crude alcohol product (200 mg). This alcohol (180 mg, 0.75 mmol) was oxidized directly with iodobenzene diacetate (266 mg, 0.83 mmol) and catalytic TEMPO (10%) in methylene chloride solvent (15 mL). The reaction mixture was quenched with aqueous sodium thiosulfate, partitioned with methylene chloride, the organic phase washed with aqueous NaHCO₃, and the organic phase concentrated in vacuo to provide the clean aldehyde product. This crude aldehyde intermediate (180 mg, 0.75 mmol) was combined with methyl (triphenylphosphoranylidene) acetate (376 mg, 1.1 mmol) in toluene (20 mL), and the reaction mixture heated at reflux. The mixture was concentrated in vacuo to a residue which was purified by flash column chromatography (SiO₂, EtOAc/hexanes) to give the desired methyl enoate. This intermediate was dissolved in tetrahydrofuran (20 mL), treated with aqueous 1N NaOH (2 mL), refluxed, the mixture cooled, acidified and extracted with diethyl ether. The organic phase was concentrated in vacuo to provide the clean enoic acid, which was then treated directly with catalytic palladium on carbon in methanol (15 mL), and hydrogenated at 1 atmosphere with a hydrogen-filled balloon. The reaction mixture was filtered over celite and concentrated in vacuo to provide the clean crude acid which is defined as Compound E in Scheme 3. Compound E (130 mg, 0.48 mmol) was converted into EXAMPLE 9 in a manner similar to EXAMPLE 1 and illustrated in Scheme 1 using anthranilic acid directly in the amide coupling reaction. The product was purified via preparative RPHPLC to give the desired product. ¹H NMR (CDCl₃, 500 MHz) δ 10.89 (s, 1H), 8.76 (d, 1H), 8.09 (d, 1H), 7.68 (d, 2H), 7.61 (t, 1H), 7.57 (s, 1H), 7.32 (d, 1H), 7.13 (m, 3H), 3.93 (s, 3H), 2.91 (m, 1H), 2.44 (t, 2H), 1.79 (m, 4H), 1.35 (d, 3H); LCMS m/z 390 (M⁺−1).

### Example 10

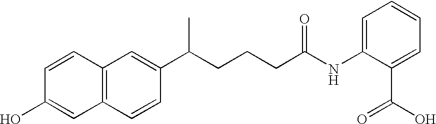

EXAMPLE 10 was prepared from EXAMPLE 9 (34 mg, 0.087 mmol) in a manner similar to EXAMPLE 6 and illustrated in Scheme 3 using boron tribromide. The product was purified via preparative RPHPLC to give the desired product. ¹H NMR (CD₃OD, 500 MHz) δ 8.55 (d, 1H), 8.08 (d, 1H), 7.64 (d, 1H), 7.58 (d, 1H), 7.33 (m, 2H), 7.29 (d, 1H), 7.14 (t, 1H), 7.06 (s, 1H), 7.03 (d, 1H), 2.88 (m, 1H), 2.43 (t, 2H), 1.76 (m, 3H), 1.62 (m, 1H), 1.33 (d, 3H); LCMS m/z 376 (M⁺−1).

### Chiral Resolution of Compound E as its Methyl Ester

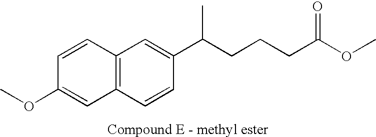

The racemic methyl ester of Compound E in Scheme 3 was resolved into its enantiomers: Preparative Chiralcel OJ column; isocratic elution with 35% isopropanol-heptane; 9 mL/min; UV=217 nm; retention times of 20.79 and 28.14 minutes. Upon demethylation of the methyl esters with potassium trimethylsilanolate in THF, the subsequent single enantiomers of Compound E in Scheme 3 were converted into single enantiomers of EXAMPLES 9 and 10 under conditions described above.

### Example 11

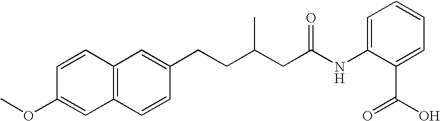

Commercially available nabumetone (600 mg, 2.63 mmol) was combined with methyl (triphenylphosphoranylidene) acetate (1.23 g, 3.68 mmol) in toluene (50 mL), and the reaction mixture heated at 160° C. in a sealed tube for 16 h. The mixture was cooled, concentrated in vacuo, and the residue was purified by flash column chromatography (SiO₂, EtOAc/hexanes) to give the desired methyl enoate as a (1:1) mixture of cis/trans olefin isomers. This material (660 mg, 2.6 mmol) was dissolved in tetrahydrofuran (50 mL), treated with aqueous 1N NaOH (5.2 mL), refluxed, the mixture cooled, acidified and extracted with diethyl ether. The organic phase was concentrated in vacuo to provide the clean enoic acid, which was then treated directly with catalytic palladium on carbon in methanol (30 mL), and hydrogenated at 1 atmosphere with a hydrogen-filled balloon. The reaction mixture was filtered over celite and concentrated in vacuo to provide the clean crude acid which is defined as Compound F in Scheme 3. Compound F (90 mg, 0.33 mmol) was converted into EXAMPLE 11 in a manner similar to EXAMPLE 1 and illustrated in Scheme 1 using anthranilic acid directly in the amide coupling reaction. The product was purified via preparative RPHPLC to give the desired product. ¹H NMR (CDCl₃, 500 MHz) δ 10.89 (s, 1H), 8.79 (d, 1H), 8.10 (d, 1H), 7.66 (m, 2H), 7.62 (t, 1H), 7.57 (s, 1H), 7.31 (m, 1H), 7.13 (m, 3H), 3.93 (s, 3H), 2.90 (m, 1H), 2.79 (m, 1H), 2.54 (m, 1H), 2.33 (m, 1H), 2.22 (m, 1H), 1.83 (m, 1H), 1,67 (m, 1H), 1.13 (d, 3H); LCMS m/z 390 (M⁺−1).

### Example 12

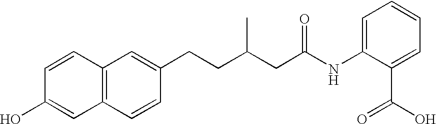

EXAMPLE 12 was prepared from EXAMPLE 11 (17 mg, 0.044 mmol) in a manner similar to EXAMPLE 6 and illustrated in Scheme 3 using boron tribromide. The product was purified via preparative RPHPLC to give the desired product. ¹H NMR (CD₃OD, 500 MHz) δ 8.58 (d, 1H), 8.10 (d, 1H), 7.61 (d, 1H), 7.55 (m, 3H), 7.25 (d, 1H), 7.16 (t, 1H), 7.06 (s, 1H), 7.03 (m, 1H), 2.85 (m, 1H), 2.74 (m, 1H), 2.55 (m, 1H), 2.33 (m, 1H), 2.14 (m, 1H), 1.83 (m, 1H), 1,67 (m, 1H), 1.11 (d, 3H); LCMS m/z 376 (M⁺−1).

### Example 13

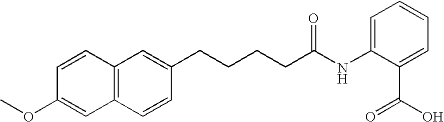

EXAMPLE 13 was prepared from commercially available 6-methoxy-2-naphthaldehyde and methyl (triphenylphosphoranylidene) acetate via methods known to those skilled in the art, and in a manner similar to the examples above and illustrated in Scheme 2 for the synthesis of Compound B. The desired product was purified via preparative RPHPLC. ¹H NMR (CDCl₃, 500 MHz) δ 11.03 (s, 1H), 8.78 (d, 1H), 8.12 (d, 1H), 7.68 (m, 2H), 7.62 (t, 1H), 7.58 (s, 1H), 7.32 (d, 1H), 7.15 (m, 2H), 7.13 (s, 1H), 3.94 (s, 3H), 2.83 (t, 2H), 2.52 (t, 2H), 1.86 (m, 4H); LCMS m/z 376 (M⁺−1).

### Example 14

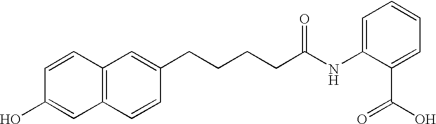

EXAMPLE 14 was prepared from EXAMPLE 13 (11 mg, 0.028 mmol) in a manner similar to EXAMPLE 6 and illustrated in Scheme 3 using boron tribromide. The product was purified via preparative RPHPLC to give the desired product. ¹H NMR (CD₃OD, 500 MHz) δ 8.55 (d, 1H), 8.08 (d, 1H), 7.61 (d, 1H), 7.54 (m, 3H), 7.24 (d, 1H), 7.13 (t, 1H), 7.05 (s, 1H), 7.01 (m, 1H), 2.77 (t, 2H), 2.48 (t, 2H), 1.79 (m, 4H); LCMS m/z 362 (M⁺−1).

### Example 15

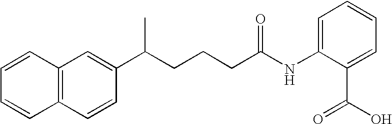

EXAMPLE 15 was prepared from commercially available 2-bromonaphthalene in a manner similar to EXAMPLE 9 and illustrated in Scheme 3 for the conversion of Compound C to Compound E. The desired product was purified via preparative RPHPLC. ¹H NMR (CD₃OD, 500 MHz) δ 8.53 (d, 1H), 8.07 (d, 1H), 7.78 (m, 3H), 7.64 (s, 1H), 7.53 (t, 1H), 7.40 (m, 3H), 7.13 (t, 1H), 2.93 (m, 1H), 2.42 (t, 2H), 1.76 (m, 3H), 1.62 (m, 1H), 1.35 (d, 3H); LCMS m/z 360 (M⁺−1).

### Example 16

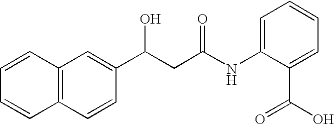

Acetic acid (1.15 g, 19.2 mmol) in 140 mL of tetrahydrofuran was cooled to −78° C., and treated with lithium diisopropylamide (1.8 M, 22.2 mL, 40 mmol). The mixture was maintained for 30 min, and then commercially available 2-naphthaldehyde (2.5 g, 16.0 mmol) was added as a solution in 20 mL of tetrahydrofuran. The mixture was warmed to room temperature, aged for 3 h, partitioned between water and diethyl ether, the aqueous phase acidified with 2N HCl to pH 2, and extracted with ethyl acetate. The organic phase was concentrated in vacuo to provide the clean hydroxy acid (1.6 g). This acid intermediate (240 mg, 1.11 mmol) was then diluted into tetrahydrofuran (10 mL), cooled to 0° C., and treated with chlorodimethoxytriazine (215 mg, 1.22 mmol) and N-methyl morpholine (123 mg, 1.22 mmol). The reaction mixture was aged for 1 h, then treated with anthranilic acid (393 mg, 2.87 mmol), aged 30 min, and warmed to room temperature overnight. The mixture was partitioned between water and ethyl acetate, and the organic phase was concentrated in vacuo to provide a residue which was purified via preparative RPHPLC to give the desired product. ¹H NMR (CD₃OD, 500 MHz) δ 8.59 (d, 1H), 8.06 (d, 1H), 7.89 (s, 1H), 7.83 (m, 2H), 7.82 (m, 3H), 7.54 (m, 2H), 7.45 (m, 2H), 7.13 (t, 1H), 5.37 (m, 1H), 2.88 (m, 2H); LCMS m/z 334 (M⁺−1).

### Example 17

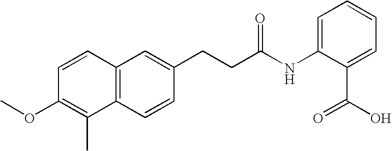

Commercially available benzyl anthranilate (1.0 g, 4.41 mmol) in 10 mL of methylene chloride was cooled to 0° C., and treated with triethylamine (3.1 mL, 22.0 mmol), followed by acryolyl chloride (725 uL, 8.8 mmol). The reaction mixture was warmed to room temperature, partitioned between water and methylene chloride, and the organic phase was separated and concentrated in vacuo. The residue was purified by flash column chromatography (SiO₂, EtOAc/hexanes) to give the desired acrylamide. This acrylamide benzyl ester (100 mg, 0.37 mmol) was then combined with commercially available 6-bromo-(1-chloromethyl)-2-methoxynaphthalene (103 mg, 0.36 mmol), diluted into dry degassed DMF (5 mL), treated with powdered sieves, triethylamine (0.15 mL, 1.08 mmol), AgOAc (180 mg, 1.08 mmol), palladium acetate (20 mg), P(O-tolyl)₃ (40 mg), and the mixture heated to 100° C. for 15 h in a sealed tube. The reaction mixture was partitioned between water and ethyl acetate, and the organic phase was filtered over celite, concentrated in vacuo to provide a residue which was passed through a plug of silica gel (EtOAc-hexane), concentrated in vacuo, and purified via preparative RPHPLC. The material was further purified by PTLC (SiO₂, 2×1500 um, 25% DMK-hexane). This intermediate was treated with catalytic palladium hydroxide on carbon in (1:1) methanol-methylene chloride (10 mL), and hydrogenated at 1 atmosphere with a hydrogen-filled balloon for 1 h. The reaction mixture was filtered over celite, concentrated in vacuo, passed through a plug of silica gel (EtOAc-hexane then 10% MeOH—CH₂Cl₂), concentrated in vacuo, and purified via preparative RPHPLC to give the desired product:

LCMS m/z 364 (M⁺+1).

### Example 18

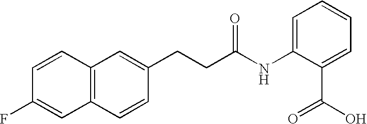

Commercially available 6-bromo-2-aminonaphthalene (100 mg, 0.45 mmol) in 4 mL of HF-pyridine was treated with sodium nitrite (101 mg, 1.35 mmol), aged to a thickly turbid mixture after 2 h, heated in a sealed tube at 85° C. for 2 h, the mixture cooled, and partitioned between chloroform and water. The organic phase was separated and concentrated in vacuo to provide the clean 6-bromo-2-fluoronaphthalene product. EXAMPLE 18 was prepared from this 6-bromo-2-fluoronaphthalene intermediate in a manner similar to EXAMPLE 17 above and illustrated in Scheme 5. The desired product was purified via preparative RPHPLC. ¹H NMR (CD₃OD, 600 MHz) δ 8.54 (d, 1H), 8.05 (dd, 1H), 7.81 (dd, 1H), 7.66-7.38 (m, 2H), 7.54 (dt, 1H), 7.46-7.44 (m, 2H), 7.24 (dt, 1H), 7.12 (t, 1H), 3.20 (t, 2H), 2.83 (t, 2H); LCMS m/z 338 (M⁺+1).

### Example 19

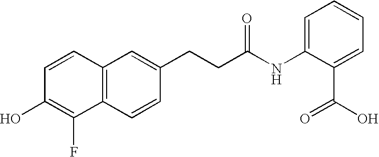

Commercially available 6-bromo-2-hydroxynaphthalene (3 g, 13.5 mmol) in 100 mL of methanol was treated with SELECTFLUOR (4.1 g, 11.5 mmol) and ACCUFLUOR (0.63 g, 1.9 mmol) at 0° C., warmed to room temperature, aged for 15 h, and the mixture concentrated in vacuo. The residue was purified by flash column chromatography (SiO₂, diethyl ether/hexanes) to give the desired 6-bromo-1-fluoro-2-hydroxynaphthalene product. EXAMPLE 19 was prepared from this intermediate in a manner similar to EXAMPLE 17 above and illustrated in Scheme 5. The desired product was purified via preparative RPHPLC. ¹H NMR (CD₃OD, 600 MHz) δ 8.51 (d, 1H), 8.02 (dd, 1H), 7.82 (d, 1H), 7.61 (s, 1H), 7.51 (t, 1H), 7.42 (d, 1H), 7.38 (dd, 1H), 7.13-7.08 (m, 2H), 3.14 (t, 2H), 2.79 (t, 2H); LCMS m/z 354 (M⁺+1).

### Example 20

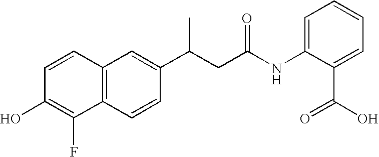

As shown in Scheme 6, the naphthyl acrylamide benzyl ester that was prepared in a manner similar to the above examples, (100 mg, 0.23 mmol) was dissolved in methylene chloride (5 mL), treated with imidazole (40 mg, 0.59 mmol), followed by t-butyldimethylsilyl chloride (55 mg, 0.36 mmol), and the reaction mixture was aged for 15 h. The crude reaction mixture was purified by PTLC (SiO₂, 25% DMK-hexane) to provide the desired silyl ether. This intermediate (25 mg, 0.045 mmol) in diethyl ether (1 mL) was added at −78° C. to a reaction mixture consisting of CuI (17 mg, 0.09 mmol) in diethyl ether (1 mL) that was treated with methyl lithium (1.6 M Et₂O, 112 uL, 0.18 mmol) at 0° C., cooled to −78° C., and had been treated with trimethylsilyl chloride (13 uL, 0.09 mmol) in diethyl ether (0.5 mL). The reaction mixture was warmed to room temperature, then 32° C. overnight. [The addition of 5 molar equivalents excess triethylamine to the final reaction mixture dramatically increases the rate of reaction and efficiency of product formation.] This intermediate (7 mg, 0.012 mmol) in tetrahydrofuran (0.5 mL) was treated twice with tetrabutylammonium fluoride (1 M THF, 62 uL, 0.062 mmol), and after 1 h the mixture was partitioned between saturated aqueous NH₄Cl and ethyl acetate. The organic phase was separated, concentrated in vacuo, and the residue was purified via preparative RPHPLC. The product was diluted into (1:1) methanol-methylene chloride (4 mL), treated with catalytic palladium hydroxide on carbon, and the mixture hydrogenated at 1 atmosphere with a hydrogen-filled balloon for 2 h. The reaction mixture was filtered over celite, concentrated in vacuo, and purified via preparative RPHPLC to give the desired product. ¹H NMR (CD₃OD, 600 MHz) δ 8.45 (d, 1H), 8.01 (dd, 1H), 7.83 (d, 1H), 7.63 (s, 1H), 7.59-7.43 (m, 3H), 7.12-7.07 (m, 2H), 3.49-3.46 (m, 1H), 2.80-2.72 (m, 2H), 1.41 (d, 3H); LCMS m/z 368 (M⁺+1).

### Example 21

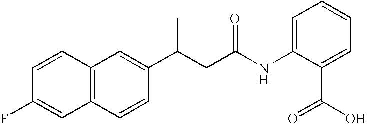

EXAMPLE 21 was prepared in a manner similar to EXAMPLE 20 above and illustrated in Scheme 6, beginning with 6-bromo-2-fluoronaphthalene described in EXAMPLE 18. The desired product was purified via preparative RPHPLC. ¹H NMR (CD₃OD, 600 MHz) δ 8.44 (d, 1H), 7.99 (dd, 1H), 7.79 (dd, 1H), 7.74-7.72 (m, 2H), 7.48-7.45 (m, 2H), 7.40 (dd, 1H), 7.20 (dt, 1H), 7.06 (t, 1H), 3.49 (q, 1H), 2.79 (m, 2H), 1.41 (d, 3H); LCMS m/z 352 (M⁺+1).

### Chiral Resolution of EXAMPLE 21 as its Benzyl Ester

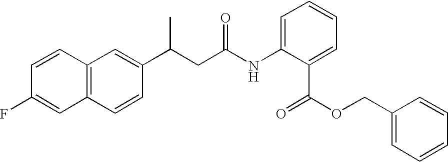

### Example 21-benzyl ester

The racemic benzyl ester intermediate of EXAMPLE 21 was resolved into its enantiomers: Preparative Chiralpak AD column; isocratic elution with 30% ethanol-hexane; retention times of 17.8 minutes (99.9% ee) and 21.3 minutes (97.2% ee). Upon hydrogenolysis of the benzyl esters with Pearlman's catalyst as in EXAMPLE 20, the subsequent single enantiomers of EXAMPLE 21 were isolated.

### Example 22

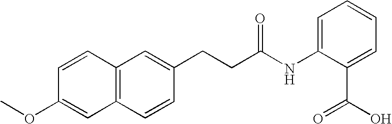

EXAMPLE 22 was prepared from commercially available 2-bromo-6-methoxynaphthalene in a manner similar to EXAMPLE 17 and illustrated in Scheme 5. The desired product was purified via preparative RPHPLC. ¹H NMR (CD₃OD, 600 MHz) δ 8.53 (d, 1H), 8.03 (dd, 1H), 7.66 (d, 1H), 7.63 (d, 1H), 7.61 (s, 1H), 7.52 (t, 1H), 7.33 (dd, 1H), 7.15 (d, 1H), 7.11 (t, 1H), 7.05 (dd, 1H), 3.85 (s, 3H), 3.15 (t, 2H), 2.79 (t, 2H); LCMS m/z 371.99 (M⁺+Na).

### Example 23

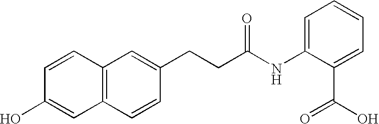

EXAMPLE 23 was prepared from commercially available 6-bromo-2-naphthol in a manner similar to EXAMPLE 17 and illustrated in Scheme 5. The desired product was purified via preparative RPHPLC: ¹H NMR (CD₃OD, 500 MHz) δ 8.46 (d, 1H), 7.97 (dd, 1H), 7.55-7.45 (m, 4H), 7.22 (d, 1H), 6.97 (t, 1H), 6.97-6.93 (m, 2H), 3.06 (t, 2H), 2.71 (t, 2H); LCMS m/z 336 (M⁺+1).

### Example 24

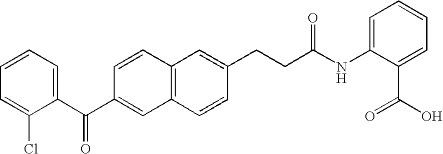

EXAMPLE 24 was prepared from commercially available 6-bromo-2-(2-chlorobenzoyl)naphthalene in a manner similar to EXAMPLE 17 and illustrated in Scheme 5. The desired product was characterized by: ¹H NMR (DMSO-d6, 500 MHz) δ 10.29 (s, 1H), 7.80 (s, 1H), 7.59 (d, 1H), 7.31 (d, 1H), 7.15)d, 1H), 7.08 (d, 1H), 6.76-6.62 (m, 8H), 6.26 (t, 1H), 3.18 (s, 1H), 2.31 (t, 2H), 1.95 (t, 2H); LCMS m/z 457.96 (M⁺+1).

### Example 25

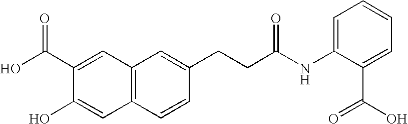

EXAMPLE 25 was prepared from commercially available 7-bromo-3-hydroxy-2-naphthoic acid in a manner similar to EXAMPLE 17 and illustrated in Scheme 5. The desired product was characterized by: ¹H NMR (CD₃OD, 500 MHz) δ 8.45 (d, 1H), 8.37 (s, 1H), 7.96 (d, 1H), 7.62 (s, 1H), 7.55 (d, 1H), 7.45 (t, 1H), 7.35 (d, 1H), 7.11 (s, 1H), 7.04 (t, 1H), 3.08 (t, 2H), 2.73 (t, 2H); LCMS m/z 380 (M⁺+1).

### Example 26

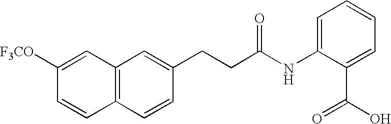

EXAMPLE 26 was prepared from 2-bromo-7-(trifluoromethoxy)naphthalene in a manner similar to EXAMPLE 17 and illustrated in Scheme 5. The 2-bromo-7-(trifluoromethoxy)naphthalene was prepared from 2-bromo-7-(trifluoromethoxy)-1,4-dihydro-1,4-epoxynaphthalene [ref: Schlosser, M., Castgnetti, E., Eur. J. Org. Chem. 2001, 3991-3997] and 3 equivalents of NaI, dissolved in dry CH₃CN (0.1M reaction concentration), followed by the addition of 3 equivalents of trimethylsilyl chloride. The reaction mixture was stirred for 2-3 h, quenched with 5% Na₂SO₃, and extracted with ether. The ether solution was washed with 5% Na₂SO₃, brine, and dried over Na₂SO₄. The crude product was chromatographed (SiO₂, hexanes) to give 2-bromo-7-(trifluoromethoxy)naphthalene. EXAMPLE 26 was purified via preparative RPHPLC. ¹H NMR (CD₃OD, 600 MHz) δ 8.54 (d, 1H), 8.05 (dd, 1H), 7.90 (d, 1H), 7.85 (d, 1H), 7.77 (s, 1H), 7.67 (s, 1H), 7.55 (t, 1H), 7.49 (dd, 1H), 7.31 (dd, 1H), 7.12 (t, 1H), 3.22 (t, 2H), 2.85 (t, 2H); LCMS m/z 404 (M⁺+1).

### Example 27

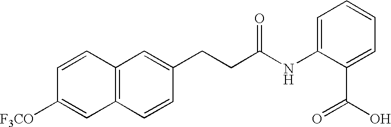

EXAMPLE 27 was prepared from 2-bromo-6-(trifluoromethoxy)naphthalene in a manner similar to EXAMPLE 17 and illustrated in Scheme 5. The 2-bromo-6-(trifluoromethoxy)naphthalene was prepared according to the conditions for 2-bromo-7-(trifluoromethoxy)naphthalene described above in EXAMPLE 26. EXAMPLE 27 was purified via preparative RPHPLC. ¹H NMR (CD₃OD, 600 MHz) δ 8.52 (d, 1H), 8.02 (dd, 1H), 7.86 (d, 1H), 7.81 (d, 1H), 7.77 (s, 1H), 7.68 (s, 1H), 7.53-7.48 (m, 2H), 7.32 (d, 1H), 7.11 (t, 1H), 3.20 (t, 2H), 2.83 (t, 2H); LCMS m/z 404 (M⁺+1).

### Example 28

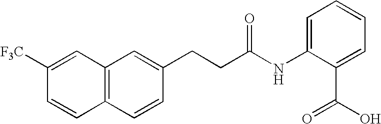

EXAMPLE 28 was prepared from 2-bromo-7-(trifluoromethyl)naphthalene in a manner similar to EXAMPLE 17 and illustrated in Scheme 5. The 2-bromo-7-(trifluoromethyl)naphthalene was prepared in the following manner:

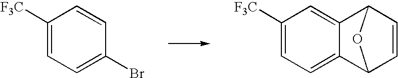

To 25 mL of tetrahydrofuran at −78° C. was added n-butyllithium (13.9 mL, 22.2 mmol) followed by diisopropylamine (3.1 mL, 22.2 mmol). The resultant mixture was stirred at −78° C. for 10 minutes, and furan (24 mL, 330 mmol) was added slowly. 4-Bromobenzotrifluoride (5 g, 22.2 mmol) was added to the reaction mixture as a solution in 10 mL of tetrahydrofuran, the cold bath was removed, and the mixture allowed to warm to ambient temperature over 2.5 h. Water was added, the mixture poured into hexanes, and the organic layer washed successively with two portions of 1N HCl and one portion of brine. The organic layer was dried over magnesium sulfate, concentrated in vacuo, and the oily residue purified by flash column chromatography (SiO₂, 5% ethyl acetate/hexanes) to give 6-(trifluoromethyl)-1,4-dihydro-1,4-epoxynaphthalene.

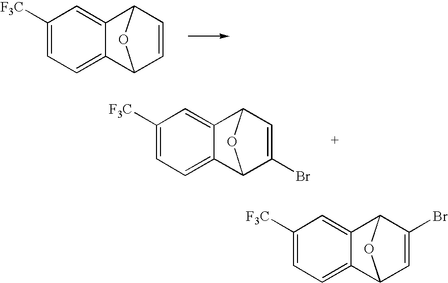

This 6-(trifluoromethyl)-1,4-dihydro-1,4-epoxynaphthalene (380 mg, 1.79 mmol) and sodium carbonate (200 mg, 1.89 mmol) were combined in 11 mL of carbon tetrachloride and heated to 70° C. Bromine (288 mg, 1.80 mmol) was added drop-wise as a solution in 3 mL of carbon tetrachloride, and the resultant mixture heated at 80° C. for 10 minutes. The pale yellow solution was cooled, filtered through a pad of sodium sulfate, and concentrated in vacuo. The oily residue obtained was suspended in 4 mL of tetrahydrofuran and added to a suspension of potassium tert-butoxide (638 mg, 5.4 mmol) in 5 mL of tetrahydrofuran at 50° C. After heating at 50° C. for 24 h, the mixture was cooled, poured into hexanes, and washed successively with two portions of water and one portion of brine. The organic layer was dried over magnesium sulfate, concentrated in vacuo, and purified by preparative TLC (SiO₂, 5% ethyl acetate/hexanes) to give a 2:1 mixture of vinyl bromide regioisomers.

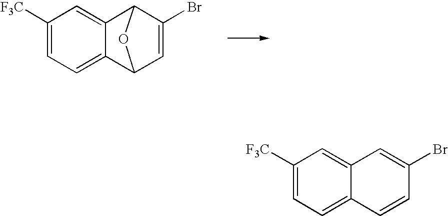

2-Bromo-7-(trifluoromethyl)-1,4-dihydro-1,4-epoxynaphthalene and sodium iodide (3 equiv.) were dissolved in dry acetonitrile (0.1 M), and trimethylsilyl chloride (3 equiv.) was added. The reaction mixture was stirred for 3-4 h, poured into hexanes, and the organic layer washed successively with two portions of water and one portion of brine. The organic layer was dried over magnesium sulfate, concentrated in vacuo, and the residue purified by flash column chromatography (SiO₂, 5% ethyl acetate/hexanes) to give 2-bromo-7-(trifluoromethyl)naphthalene.

EXAMPLE 28 was purified via preparative RPHPLC. ¹H NMR (CD₃OD, 600 MHz) δ 8.49 (d, 1H), 8.03 (s, 1H), 7.87 (d, 1H), 7.75-7.70 (m, 2H), 7.51 (d, 1H), 7.43-7.34 (m, 3H), 6.99 (t, 1H), 3.11 (t, 2H), 2.74 (t, 2H); LCMS m/z 388 (M⁺−1).

### Example 29

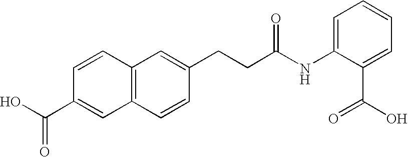

EXAMPLE 29 was prepared from commercially available 6-bromo-2-naphthoic acid in a manner similar to EXAMPLE 17 and illustrated in Scheme 5. The desired product was purified via preparative RPHPLC: ¹H NMR (CD₃OD, 500 MHz) δ 8.48-8.45 (d, 3H), 7.97 (d, 1H), 7.91 (d, 1H), 7.85 (d, 1H), 7.73 (s, 1H), 7.46-7.43 (m, 2H), 7.05 (t, 1H), 3.17 (t, 2H), 2.79 (t, 2H); LCMS m/z 363 (M⁺+1).

### Example 30

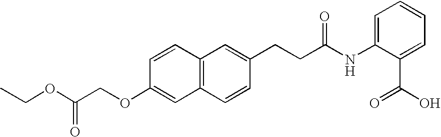

EXAMPLE 30 was prepared from commercially available ethyl 2-(2-(6-bromo)naphthoxy)acetate in a manner similar to EXAMPLE 17 and illustrated in Scheme 5. The desired product was purified via preparative RPHPLC: ¹H NMR (DMSO-d₆, 500 MHz) δ 10.27 (s, 1H), 7.60 (d, 1H), 7.09 (d, 1H), 6.90 (d, 1H), 6.85-6.83 (m, 2H), 6.70 (t, 1H), 6.53 (d, 1H), 6.36 (d, 1H), 6.31 (dd, 1H), 6.28 (t, 1H), 3.99 (s, 2H), 3.32 (q, 2H), 2.22 (t, 2H), 1.93 (t, 2H), 0.35 (t, 3H); LCMS m/z 422 (M⁺+1).

### Example 31

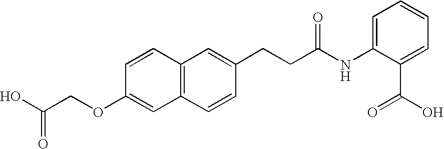

EXAMPLE 31 was prepared from the diester intermediate in EXAMPLE 30 via saponification with LiOH followed by hydrogenation under conditions described above. The desired product was purified via preparative RPHPLC: ¹H NMR (CD₃OD, 500 MHz) δ 8.44 (d, 1H), 7.95 (dd, 1H), 7.57 (t, 2H), 7.54 (s, 1H), 7.42 (t, 1H), 7.26 (dd, 1H), 7.25-7.00 (m, 3H), 3.22 (2, 3H), 3.06 (t, 2H), 2.71 (t, 2H); LCMS m/z 393.98 (M⁺+1).

### Example 32

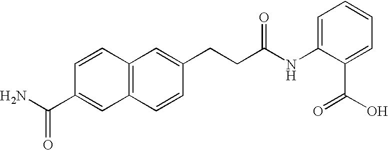

EXAMPLE 32 was prepared from the benzyl ester acrylamide intermediate in EXAMPLE 29 (24 mg, 0.05 mmol). This material was diluted into methylene chloride (1 mL), chilled to 0° C., and combined with triethylamine (20 uL, 0.15 mmol) and methanesulfonyl chloride (10 uL, 0.10 mmol). The reaction mixture was aged for 0.5 h, warmed to room temperature, and bubbled through with ammonia gas for 5 min. After 30 minutes, the mixture was concentrated in vacuo, and the desired product was purified via preparative RPHPLC. This intermediate was hydrogenated in a similar manner as described in the examples above to provide the desired product. ¹H NMR (CD₃OD, 500 MHz) δ 8.47 (d, 1H), 8.31 (d, 1H), 7.97 (d, 1H), 7.85-7.80 (m, 3H), 7.73 (s, 1H), 7.44 (s, 2H), 7.05 (d, 1H), 3.17 (t, 1H), 2.80 (t, 2H); LCMS m/z 362.99 (M⁺+1).

### Example 33

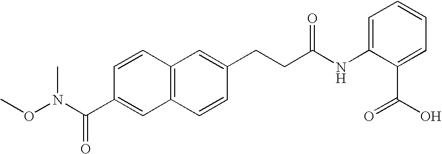

EXAMPLE 33 was prepared from the benzyl ester acrylamide intermediate in EXAMPLE 29 (100 mg, 0.22 mmol). This material was diluted into methylene chloride (4 mL), and combined with diisopropylethylamine (110 uL, 0.66 mmol), EDCl (288 mg, 0.33 mmol), N,N-methoxy(methyl)amine hydrochloride (32 mg, 0.33 mmol), and the reaction mixture was aged for 15 h. The mixture was partitioned between saturated aqueous ammonium chloride and ethyl acetate, the organic phase separated and concentrated in vacuo. The desired product was purified via preparative RPHPLC. This intermediate was hydrogenated in a similar manner as described in the examples above to provide the desired product: ¹H NMR (CD₃OD, 500 MHz) δ 8.44 (d, 1H), 8.03 (s, 1H), 7.93 (dd, 1H), 7.78 (d, 1H), 7.73 (d, 1H), 7.67 (s, 1H), 7.55 (dd, 1H), 7.43 (dt, 1H), 7.40 (dd, 1H), 3.49 (s, 3H), 3.29 (s, 3H), 3.13 (t, 2H), 2.75 (t, 2H); LCMS m/z 407 (M⁺+1).

### Example 34

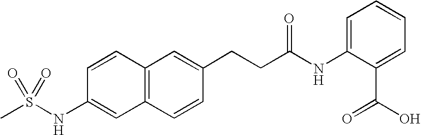

EXAMPLE 34 was prepared from commercially available 6-bromo-2-aminonaphthalene by first sulfonylation of the amine under standard conditions known to those skilled in the art, and subsequent homologation in a manner similar to EXAMPLE 17 and illustrated in Scheme 5. The desired product was purified via preparative RPHPLC. ¹H NMR (CD₃OD, 600 MHz) δ 8.52 (d, 1H), 8.02 (dd, 1H), 7.74 (d, 1H), 7.70 (d, 1H), 7.67 (s, 1H), 7.64 d, 1H), 7.52 (t, 1H), 7.40 (dd, 1H), 7.33 (dd, 1H), 7.10 (t, 1H), 3.18 (t, 2H), 2.81 (t, 2H); LCMS m/z 413 (M⁺+1).

### Example 35

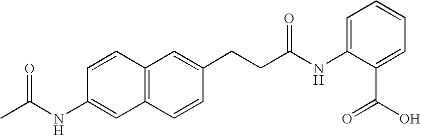

EXAMPLE 35 was prepared from commercially available 6-bromo-2-aminonaphthalene by first acetylation of the amine under standard conditions known to those skilled in the art, and subsequent homologation in a manner similar to EXAMPLE 17 and illustrated in Scheme 5. The desired product was purified via preparative RPHPLC. ¹H NMR (CD₃OD, 600 MHz) δ 8.52 (d, 1H), 8.01 (d, 1H), 8.03 (dd, 1H), 7.69 (t, 2H), 7.64 (s, 1H), 7.51 (t, 1H), 7.47 (dd, 1H), 7.37 (dd, 1H), 7.09 (t, 1H), 3.17 (t, 2H), 2.81 (t, 2H), 2.14 (s, 3H); LCMS m/z 377 (M⁺+1).

### Example 36

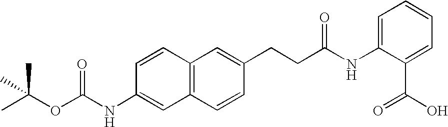

EXAMPLE 36 was prepared from commercially available 6-bromo-2-aminonaphthalene by first carbamoylation of the amine with di-tert-butyl dicarbonate under standard conditions known to those skilled in the art, and subsequent homologation in a manner similar to EXAMPLE 17 and illustrated in Scheme 5. The desired product was purified via preparative RPHPLC. ¹H NMR (CD₃OD, 600 MHz) δ 8.54 (d, 1H), 8.05 (d, 1H), 7.91 (s, 1H), 7.67 (d, 1H), 7.65 (d, 1H), 7.61 (s, 1H), 7.53 (t, 1H), 7.40 (dd, 1H), 7.34 (dd, 1H), 7.12 (t, 1H), 3.16 (t, 2H), 2.81 (t, 2H), 1.54 (s, 9H); LCMS m/z 435 (M⁺+1).

### Example 37

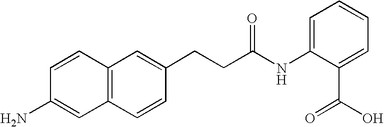

EXAMPLE 37 was prepared from EXAMPLE 36 with the use of trifluoroacetic acid under standard conditions known to those skilled in the art, and the desired product was purified via preparative RPHPLC. ¹H NMR (CD₃OD, 600 MHz) δ 8.51 (d, 1H), 8.02 (dd, 1H), 7.92 (d, 1H), 7.82 (d, 1H), 7.79 (s, 1H), 7.74 (d, 1H), 7.53-7.49 (m, 2H), 7.36 (dd, 1H), 7.11 (t, 1H), 3.22 (t, 2H), 2.84 (t, 2H).

### Example 38

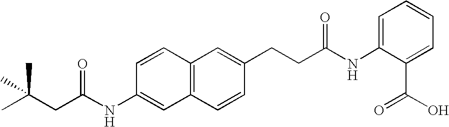

EXAMPLE 38 was synthesized from EXAMPLE 37 as its methyl ester (prepared in an analogous fashion to the benzyl ester described in the examples above and illustrated in Scheme 5) via tert-butylacetylchloride and subsequent saponification with LiOH under standard conditions known to those skilled in the art. The desired product was purified via preparative RPHPLC. ¹H NMR (CD₃OD, 500 MHz) δ 8.45 (d, 1H), 8.05 (d, 1H), 7.97 (dd, 1H), 7.64 (t, 2H), 7.59 (s, 1H), 7.43-7.40 (m, 2H), 7.32 (dd, 1H), 7.03 (t, 1H), 3.11 (t, 2H), 2.75 (t, 2H), 2.21 (s, 2H), 1.04 (s, 9H); LCMS m/z 433 (M⁺+1).

### Example 39

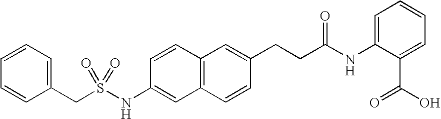

EXAMPLE 39 was synthesized from EXAMPLE 37 as its benzyl ester (described in the examples above and illustrated in Scheme 5) via benzylsulfonyl chloride under standard conditions known to those skilled in the art, and subsequent hydrogenation to provide the desired product, purified via preparative RPHPLC. ¹H NMR (C₃OD, 500 MHz) δ 8.46 (d, 1H), 7.96 (dd, 1H), 7.66-7.59 (m, 3H), 7.52 (d, 1H), 7.45 (t, 1H), 7.33 (d, 1H), 7.22-7.16 (m, 5H), 7.03 (t, 1H), 5.41 (s, 2H), 3.11 (t, 2H), 2.76 (t, 2H); LCMS m/z 489 (M⁺+1).

### Example 40

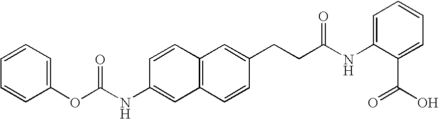

EXAMPLE 40 was prepared from EXAMPLE 37 as its benzyl ester (20 mg, 0.05 mmol) described in the examples above and illustrated in Scheme 7. This amine was diluted into pyridine (0.04 mL, 0.50 mmol), cooled to 0° C., treated with phenyl chloroformate (0.02 mL, 0.15 mmol), the reaction mixture warmed to room temperature overnight and then 40° C. for 1.5 h. The mixture was cooled, partitioned between aqueous citric acid and ethyl acetate, the organic phase separated and concentrated in vacuo. The product was purified via preparative RPHPLC. This intermediate was hydrogenated with Pearlman's catalyst in a similar manner as described in the examples above to provide the desired product. ¹H NMR (CD₃OD, 500 MHz) δ 8.47 (d, 1H), 7.98 (dd, 1H), 7.93 (s, 1H), 7.64 (d, 1H), 7.60 (d, 1H), 7.48-7.38 (m, 2H), 7.37-7.30 (m, 3H), 7.24-7.13 (m, 2H), 7.04 (t, 1H), 3.11 (t, 2H), 2.76 (t, 2H); LCMS m/z 455 (M⁺+1).

### Example 41

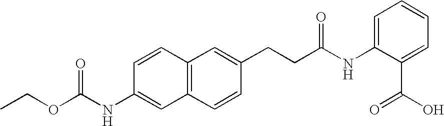

EXAMPLE 41 was synthesized from EXAMPLE 37 as its methyl ester via ethyl chloroformate under conditions described in the examples above. The desired product was purified via preparative RPHPLC: ¹H NMR (CD₃OD, 500 MHz) δ 8.46 (d, 1H), 7.97 (dd, 1H), 7.85 (s, 1H), 7.60 (t, 2H), 7.55 (s, 1H), 7.46 (t, 1H), 7.36 (dd, 1H), 7.28 (dd, 1H), 7.04 (t, 1H), 4.13 (q, 2H), 3.09 (t, 2H), 2.74 (t, 2H), 1.25 (t, 3H); LCMS m/z 407 (M⁺+1).

### Example 42

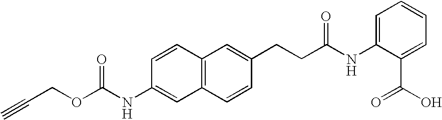

EXAMPLE 42 was synthesized from EXAMPLE 37 as its methyl ester via propargyl chloroformate under conditions described in the examples above. The desired product was purified via preparative RPHPLC. ¹H NMR (CD₃OD, 500 MHz) δ 8.46 (d, 1H), 7.97 (d, 1H), 7.87 (s, 1H), 7.68-7.59 (m, 2H), 7.56 (s, 1H), 7,45 (t, 1H), 7.38 (d, 1H), 7.29 (d, 1H), 7.04 (t, 1H), 4.71 (s, 2H), 3.09 (t, 2H), 2.85 (s, 1H), 2.74 (t, 2H); LCMS m/z 417 (M⁺+1).

### Example 43

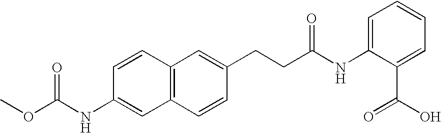

EXAMPLE 43 was synthesized from EXAMPLE 37 as its methyl ester via methyl chloroformate under conditions described in the examples above. The desired product was purified via preparative RPHPLC. ¹H NMR (CD₃OD, 500 MHz) δ 8.46 (d, 1H), 7.97 (dd, 1H), 7.86 (s, 1H), 7.61 (t, 2H), 7.56 (s, 1H), 7.46 (dt, 1H), 7.36 (dd, 1H), 7.29 (dd, 1H), 7.05 (t, 1H), 3.69 (s, 3H), 3.09 (t, 2H), 2.74 (t, 2H); LCMS m/z 393 (M⁺+1).

### Example 44

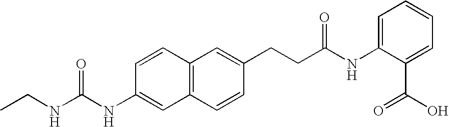

EXAMPLE 44 was synthesized from EXAMPLE 37 as its benzyl ester via ethyl isocyanate under conditions described in the examples above. The desired product was purified via preparative RPHPLC. ¹H NMR (CD₃OD, 500 MHz) δ 8.46 (d, 1H), 7.96 (d, 1H), 7.79 (s, 1H), 7.59-7.54 (m, 4H), 7.47 (t, 1H), 7.30-7.27 (m, 2H), 7.05 (t, 1H), 3.16 (q, 2H), 3.09 (t, 2H), 2.74 (t, 2H), 1.13 (t, 3H); LCMS m/z 406 (M⁺+1).

### Example 45

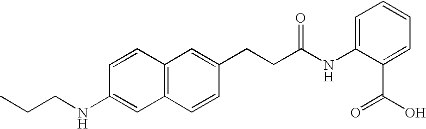

EXAMPLE 45 was prepared by reductive amination of EXAMPLE 37 as its benzyl ester (20 mg, 0.05 mmol), with propionaldehyde (10 uL, 0.08 mmol), diisopropylethylamine (30 uL, 0.15 mmol), sodium triacetoxyborohydride (21 mg, 0.10 mmol), and powdered sieves in methylene chloride (1 mL). The reaction mixture was aged 15 h, partitioned between saturated aqueous NaHCO₃ and ethyl acetate, the organic phase separated, and concentrated in vacuo. The product was purified via preparative RPHPLC (10 mg). This benzyl ester intermediate was hydrogenated with Pearlman's catalyst in a similar manner as described in the examples above to provide the desired product. ¹H NMR (CD₃OD, 500 MHz) δ 8.41 (d, 1H), 7.91 (dd, 1H), 7.81 (d, 1H), 7.73 (d, 1H), 7.67 (s, 1H), 7.42-7.39 (m, 3H), 7.89 (d, 1H), 7.01 (t, 1H), 3.27 (t, 2H), 3.11 (t, 2H), 2.73 (t, 2H), 1.68-1.63 (m, 2H), 0.94 (t, 3H); LCMS m/z 377 (M⁺+1).

### Example 46

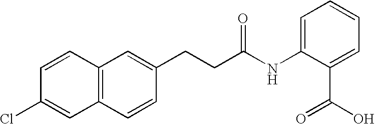

Example 46 was prepared by Sandmeyer reaction of EXAMPLE 37 as its methyl ester (30 mg, 0.09 mmol), with tert-butylnitrite (10 uL, 0.11 mmol), CuCl (445 mg, 4.5 mmol), CuCl₂ (726 mg, 5.4 mmol), and 48% (aq) HBF₄ (11 uL, 0.11 mmol) in acetonitrile (1 mL). Upon reaction completion, the reaction mixture was partitioned between saturated aqueous ammonium chloride and ethyl acetate, the organic phase separated, dried, and concentrated in vacuo. The product was purified via preparative RPHPLC (4 mg). This methyl ester intermediate was saponified with LiOH in a similar manner as described in the examples above to provide the desired product. ¹H NMR (CD₃OD, 500 MHz) δ 8.45 (d, 1H), 7.96 (d, 1H), 7.74 (s, 1H), 7.70-7.67 (m, 2H), 7.46 (t, 1H), 7.38 (d, 1H), 7.31 (dd, 1H), 7.05 (t, 1H), 3.12 (t, 2H), 2.76 (t, 2H); LCMS m/z 354 (M⁺+1).

### Example 47

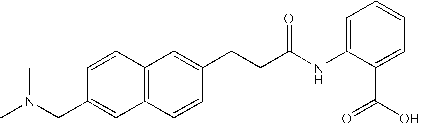

EXAMPLE 47 was prepared from commercially available 6-bromo-2-(tert-butyldimethylsilyloxymethyl)naphthalene in a manner similar to EXAMPLE 17 and illustrated in Scheme 5. The resultant benzyl ester acrylamide was desilylated under standard conditions known to those skilled in the art to provide the hydroxymethylene intermediate shown in Scheme 7. This alcohol (50 mg, 0.11 mmol) was oxidized with Dess-Martin periodinane (243 mg, 0.57 mmol) in methylene chloride (5 mL) with the addition of solid NaHCO₃ (291 mg, 2.75 mmol). Upon reaction completion, the reaction mixture was partitioned between water and ethyl acetate, the organic phase separated, dried, and concentrated in vacuo. The aldehyde product was purified via preparative RPHPLC (40 mg). This aldehyde intermediate was reductively aminated with a dimethylamine solution in THF (2 M, 3 equiv) in a similar manner as described in EXAMPLE 45 above to provide the N,N-dimethylaminomethylene naphthyl intermediate shown in Scheme 7. After preparative RPHPLC purification, this intermediate was then hydrogenated with Pearlman's catalyst in a similar manner as described in the examples above to provide the desired product. ¹H NMR (CD₃OD, 500 MHz) δ 8.46 (d, 1H), 7.96 (dd, 1H), 7.88 (s, 1H), 7.84 (d, 1H), 7.79 (d, 1H), 7.73 (s, 1H), 7.47-7.42 (m, 3H), 7.04 (t, 1H), 3.17 (t, 2H), 2.80 (s, 6H), 2.78 (t, 2H); LCMS m/z 377 (M⁺+1).

### Example 48

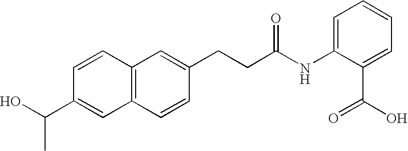

EXAMPLE 48 was prepared from the aldehyde intermediate in EXAMPLE 47 above and illustrated in Scheme 7. The benzyl ester acrylamide aldehyde (23 mg, 0.05 mmol) was diluted into dry tetrahydrofuran (2 mL), cooled to −78° C., and treated with methyl magnesium bromide (1.4 M THF, 180 uL, 0.25 mmol). The reaction mixture was warmed to room temperature, treated with an additional 5 equivalents methyl magnesium bromide (1.4 M THF, 180 uL, 0.25 mmol), aged 15 h, quenched with a few drops of glacial acetic acid, and the reaction mixture then partitioned between saturated aqueous ammonium chloride and ethyl acetate, the organic phase separated, dried, and concentrated in vacuo. The residue was purified via preparative RPHPLC to provide two products; the secondary benzylic alcohol and the eliminated vinyl naphthalene. The secondary benzylic alcohol intermediate was then hydrogenated with Pearlman's catalyst in a similar manner as described in the examples above to provide the desired product: ¹H NMR (CD₃OD, 500 MHz) δ 8.45 (d, 1H), 7.96 (dd, 1H), 7.68-7.65 (m, 3H), 7.60 (s, 1H), 7.45 (t, 1H), 7.38 (dd, 1H), 7.31 (dd, 1H), 7.04 (t, 1H), 4.86 (m, 1H), 3.11 (t, 2H), 2.76 (t, 2H), 1.42 (d, 3H); LCMS m/z 386 (M⁺+Na).

### Example 49

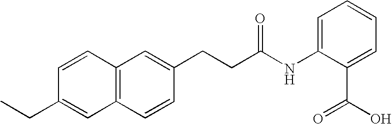

EXAMPLE 49 was prepared from the vinyl naphthalene intermediate in EXAMPLE 48 above. Thus the benzyl ester acrylamide alkenyl intermediate was hydrogenated with Pearlman's catalyst in a similar manner as described in the examples above and purified via preparative RPHPLC to provide the desired product: ¹H NMR (CD₃OD, 500 MHz) δ 8.44 (d, 1H), 7.94 (dd, 1H), 7.59-7.51 (m, 3H), 7.44-7.37 (m, 2H), 7.23 (dd, 1H), 7.19 (d, 1H), 7.00 (t, 1H), 3.06 (t, 2H), 2.71-2.63 (m, 4H), 1.18 (t, 3H); LCMS m/z 348 (M⁺+1).

### Examples 50-52

EXAMPLES 50-52 were prepared from commercially available 2,6-dibromonaphthalene in a manner similar to EXAMPLE 17 and illustrated in Scheme 5. The resultant benzyl ester acrylamide bromide intermediate (20 mg, 0.041 mmol) was diluted into DMIDO (0.5 mL), treated with 5 equivalents of CuCN (18 mg, 0.21 mmol), and the reaction mixture was heated at 160° C. for 3 h. The nitrile product was purified via preparative RPHPLC. This cyano benzyl ester acrylamide intermediate was then hydrogenated with Pearlman's catalyst in a similar manner as described in the examples above to provide the three products characterized below.

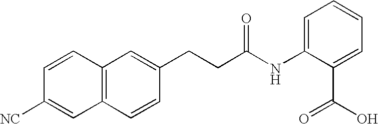

Nitrile EXAMPLE 50 was purified via preparative RPHPLC. ¹H NMR (CD₃OD, 600 MHz) δ 8.53 (d, 1H), 8.30 (s, 1H), 8.04 (dd, 1H), 7.93-7.91 (m, 2H), 7.83 (s, 1H), 7.62-7.57 (m, 2H), 7.53 (t, 1H), 7.13 (t, 1H), 3.27 (t, 2H), 2.87 (t, 2H); LCMS m/z 433 (M⁺+1).

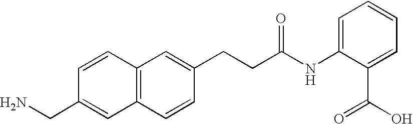

Aminomethylene EXAMPLE 51 was purified via preparative RPHPLC. ¹H NMR (CD₃OD, 600 MHz) δ 8.53 (d, 1H), 8.05 (dd, 1H), 7.90-7.87 (m, 2H), 7.84 (d, 1H), 7.78 (s, 1H), 7.53 (t, 1H), 7.50 (d, 1H), 7.12 (t, 1H), 4.26 (s, 2H), 3.23 (t, 2H), 2.86 (t, 2H); LCMS m/z 347 (M⁺−1).

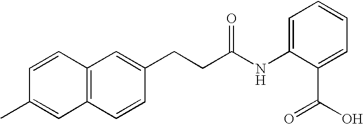

Methylnaphthalene EXAMPLE 52 was purified via preparative RPHPLC. ¹H NMR (CD₃OD, 600 MHz) δ 8.55 (d, 1H), 8.06 (d, 1H), 7.68-7.65 (m, 2H), 7.57-7.53 (m, 2H), 7.36 (dd, 1H), 7.28 (dd, 1H), 7.13 s, 1H), 3.19 (t, 2H), 2.82 (t, 2H), 2.43 (s, 3H); LCMS m/z 332 (M⁺−1).

### Example 53

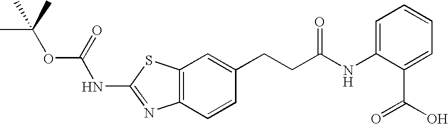

Commercially available 2-amino-6-bromobenzothiazole (2 g, 8.7 mmol) was diluted into methylene chloride (15 mL), combined with DMAP (1.1 g, 8.7 mmol) in tetrahydrofuran (10 mL), and treated with di-tert-butyl dicarbonate (2.1 g, 9.6 mmol) at 0° C. The reaction mixture was warmed to room temperature and aged overnight. The mixture was then filtered, the filtrate concentrated in vacuo, and the solid purified by flash column chromatography (Biotage, SiO₂, 5-10% EtOAc-hexane) to provide the tert-butylcarbamate-protected bromide intermediate. Commercially available methyl anthranilate was converted to the desired acrylamide using acryolyl chloride under similar conditions described in Example 17. This acrylamide methyl ester (69 mg, 0.33 mmol) was then combined with the tert-butylcarbamate-protected bromide intermediate (110 mg, 0.33 mmol), diluted into dry degassed DMF (5 mL), treated with powdered sieves, triethylamine (0.14 mL, 0.99 mmol), Bu₄NCl (92 mg, 0.33 mmol), palladium acetate (20 mg), P(O-tolyl)₃ (40 mg), and the reaction mixture heated to 100° C. for 15 h in a sealed tube. The reaction mixture was cooled to room temperature and directly purified by flash column chromatography (Biotage, SiO₂, 5-50% EtOAc-hexane) to provide the acrylamide methyl ester. This acrylamide intermediate (90 mg, 0.2 mmol) was reduced by the addition of p-toluenesulfonyl hydrazide (370 mg, 2.0 mmol) in methanol (50 mL). The reaction mixture was refluxed for 24 h, treated again with p-toluenesulfonyl hydrazide (200 mg, 1.1 mmol) and refluxed for an additional 24 h. The reaction mixture was then cooled to room temperature, and the product purified via preparative RPHPLC. The methyl ester intermediate (46 mg, 0.1 mmol) was then saponified with LiOH (1 M, 2 mL) in (3:1:1) THF-MeOH—H₂O (2 mL) for 4 h. The reaction mixture was then concentrated in vacuo, diluted with water (20 mL), extracted with chloroform (15 mL), the aqueous phase separated, acidified with conc. HCl to pH 3, and then extracted with 30% isopropanol-chloroform (50 mL). The organic partition was separated, dried over anhydrous sodium sulfate, concentrated in vacuo, and the residue was purified via preparative RPHPLC to give the desired product: ¹H NMR (DMSO-d₆, 500 MHz) δ 11.7 (s, 1H), 11.2 (s, 1H), 8.44 (d, 1H), 7.94 (d, 1H), 7.79 (s, 1H), 7.57 (d, 1H), 7.53 (d, 1H), 7.28 (dd, 1H), 7.12 (t, 1H), 3.02 (t, 2H), 2.75 (t, 2H), 1.47 (s, 9H); LCMS m/z 440 (M⁺−1).

### Example 54

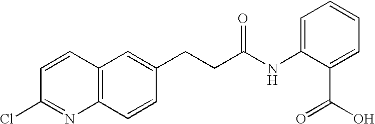

Commercially available 2,6-dihydroxyquinoline (100 mg, 0.62 mmol) was diluted into phosphorus oxychloride (2 mL), and heated at 80° C. for 1 h. The reaction mixture was cooled to room temperature, partitioned between saturated aqueous NaHCO₃ and chloroform, the organic phase separated, dried, concentrated in vacuo, and the residue purified via preparative RPHPLC. The chloroalcohol intermediate (400 mg, 2.23 mmol) was diluted into methylene chloride (5 mL), and then treated with triethylamine (620 uL, 4.5 mmol), and trifluoromethanesulfonic anhydride (591 uL, 3.4 mmol). Upon reaction completion, the reaction mixture was concentrated in vacuo, vacuum dried, and the triflate was used without purification. The chlorotriflate intermediate (69 mg, 0.22 mmol) was combined with the acrylamide benzyl ester (125 mg, 0.45 mmol) described in EXAMPLE 17, along with triethylamine (34 uL, 0.24 mmol), palladium acetate (4 mg, 2.5%), DPPP (2.5 mg, 0.006 mmol), and diluted into dry degassed DMF (5 mL). The reaction mixture was heated to 80° C. overnight in a sealed tube, cooled to room temperature, filtered, partitioned between water and ethyl acetate, and the organic phase separated, dried, and concentrated in vacuo. The residue was purified via preparative RPHPLC. This acrylamide benzyl ester chloroquinoline (15 mg, 0.034 mmol) was reduced by the addition of p-toluenesulfonyl hydrazide (82 mg, 0.44 mmol) in methanol (50 mL). The reaction mixture was refluxed for 24 h, cooled to room temperature, and the product purified via preparative RPHPLC. The benzyl ester intermediate was then saponified with LiOH in (3:1:1) THF-MeOH—H₂O in a similar manner as described in the examples above, and the acid was purified via preparative RPHPLC to provide the desired product: ¹H NMR (CDCl₃, 500 MHz) δ 10.9 (s, 1H), 8.7 (d, 1H), 8.3 (d, 1H), 8.1 (m, 2H), 8.0 (m, 3H), 7.7 (d, 2H), 7.6 (s, 1H), 7.4 (d, 2H), 7.1 (t, 1H), 3.3 (t, 2H), 2.9 (t, 2H).

### Example 55

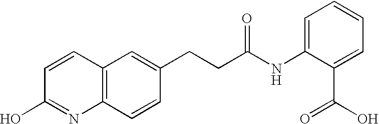

EXAMPLE 55 was prepared from the acrylamide benzyl ester chloroquinoline intermediate from EXAMPLE 54 as illustrated in Scheme 9. This chloroquinoline (15 mg, 0.034 mmol) was diluted into (1:1) 4 M HCl(aq)-dioxane, and heated at 65° C. overnight. The reaction mixture was cooled to room temperature, concentrated in vacuo, and the residue purified with PTLC (SiO₂, 30% EtOAc-hexane) by isolation of the baseline fraction. This hydroxyl intermediate was hydrogenated with Pearlman's catalyst in a similar manner as described in the examples above to provide the desired product: ¹H NMR (DMSO-d₆, 500 MHz) δ 11.6 (s, 1H), 8.4 (d, 1H), 7.9 (d, 1H), 7.8 (d, 1H), 7.5 (m, 2H), 7.4 (d, 1H), 7.2 (d, 1H), 7.1 (t, 1H), 6.5 (d, 1H), 3.0 (t, 2H), 2.8 (t, 2H); LCMS m/z 337 (M⁺+1).

### Example 56

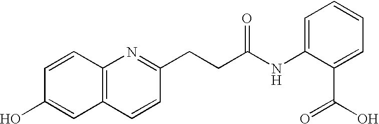

Commercially available 2,6-dihydroxyquinoline (100 mg, 0.62 mmol) was diluted into methylene chloride (3 mL), and then treated with triethylamine (86 uL, 0.62 mmol) and trifluoromethanesulfonic anhydride (105 uL, 0.62 mmol). Upon reaction completion, the reaction mixture was concentrated in vacuo, vacuum dried, and the triflate was used without purification. This hydroxy triflate intermediate (100 mg, 0.34 mmol) was combined with the acrylamide benzyl ester (192 mg, 0.68 mmol) described in EXAMPLE 17, along with triethylamine (52 uL, 0.38 mmol), palladium acetate (2.5%, 6 mg, 0.009 mmol), DPPP (4 mg, 0.009 mmol), and diluted into dry degassed DMF (5 mL). The reaction mixture was heated to 80° C. for 10 h in a sealed tube, cooled to room temperature, filtered, partitioned between water and ethyl acetate, and the organic phase separated, dried, and concentrated in vacuo. The residue was purified via preparative RPHPLC. This acrylamide benzyl ester hydroxyquinoline intermediate was hydrogenated with Pearlman's catalyst in a similar manner as described in the examples above to provide the desired product: ¹H NMR (CD₃OD, 500 MHz) δ 8.5 (d, 1H), 8.0 (m, 2H), 7.8 (s, 1H), 7.3 (m, 3H), 7.0 (m, 2H), 3.3 (t, 2H), 2.9 (t, 2H); LCMS m/z 337 (M⁺+1).

### Example 57

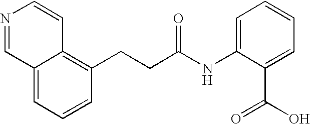

EXAMPLE 57 was prepared from commercially available 5-bromoisoquinoline in a manner similar to EXAMPLE 17 and illustrated in Scheme 5. The desired product was purified via preparative RPHPLC ¹H NMR (CD₃OD, 500 MHz) δ 9.7 (s, 1H), 8.7 (d, 1H), 8.6 (s, 1H), 8.5 (d, 1H), 8.3 (d, 1H), 8.1 (d, 1H), 8.0 (d, 1H), 7.9 (t, 1H), 7.5 (t, 1H), 7.1 (t, 1H), 3.6 (t, 2H), 2.9 (t, 2H); LCMS m/z 321 (M⁺+1).

### Example 58

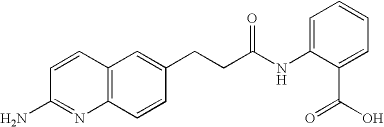

EXAMPLE 58 was prepared from commercially available 2,6-dihydroxyquinoline by first bromination with POBr₃, followed by triflation and Heck coupling in a similar manner as described in EXAMPLE 54 above and illustrated in Scheme 9. The resultant bromoquinoline acrylamide benzyl ester intermediate (12 mg, 0.025 mmol) was combined with 1.2 equivalents of benzophenone imine, excess cesium carbonate, catalytic palladium acetate and BINAP, and diluted into dry tetrahydrofuran. The reaction mixture was heated to 70° C. overnight, cooled to room temperature, diluted into a 10-fold volume of diethyl ether, filtered, and concentrated in vacuo. The crude imine intermediate was cleaved with 2N HCl(aq) in tetrahydrofuran, concentrated in vacuo and the residue purified via preparative RPHPLC. This aminoquinoline acrylamide benzyl ester intermediate (4.4 mg, 0.01 mmol) was hydrogenated with catalytic palladium on carbon in a similar manner as described in the examples above to provide the desired product. ¹H NMR (CD₃OD, 500 MHz) δ 8.6 (d, 1H), 8.3 (d, 1H), 8.0 (d, 1H), 7.9 (s, 1H), 7.7 (d, 1H), 7.6 (m, 2H), 7.2 (m, 1H), 7.0 (d, 1H), 3.2 (t, 2H), 2.9 (t, 2H); LCMS m/z 336 (M⁺+1).

### Example 59

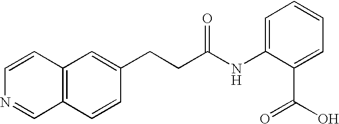

Commercially available 5-bromoindanone (5.1 g, 24.3 mmol) was diluted into methanol (150 mL), cooled to 0° C., and treated with sodium borohydride (1.8 g, 48.6 mmol). The reaction mixture was warmed to room temperature, aged overnight, and then partitioned between water and methylene chloride, the organic phase separated, dried and concentrated in vacuo. The clean crude alcohol (5.0 g, 97%) was isolated and used in the next step without purification. This hydroxybromoindane (5.04 g, 23.6 mmol) was diluted into toluene (100 mL), treated with catalytic p-toluenesulfonic acid (400 mg), and the reaction mixture refluxed under Dean-Stark trap conditions for 6 h. The mixture was cooled to room temperature, extracted with saturated aqueous sodium bicarbonate, and the organic phase separated, dried and concentrated in vacuo. The clean crude bromoindene (4.6 g, 100%) was isolated as an oil and used in the next step without purification. This bromoindene (4.5 g) was diluted into (1:1) methanol-methylene chloride (150 mL), chilled to −78° C., and treated with ozone for 30 minutes, removed from the ozonator, warmed to room temperature, and treated with solid sodium bicarbonate (2.5 g) and dimethylsulfide (3 mL). The reaction mixture was aged for 14 h, treated with 78% ammonium hydroxide in water (30 mL), and the mixture maintained at room temperature overnight. The reaction mixture was then concentrated in vacuo, re-dissolved in ethyl acetate, washed with saturated aqueous sodium bicarbonate, and the organic phase separated, dried and concentrated in vacuo. The crude product was purified by flash column chromatography (Biotage, SiO₂, 20% EtOAc-heptane) to provide the solid bromoisoquinoline. EXAMPLE 59 was prepared from this bromoisoquinoline by first Heck coupling in a similar manner as described in EXAMPLE 53 above and illustrated in Scheme 8. The resultant isoquinoline acrylamide methyl ester intermediate was saponified with LiOH, and the acid reduced with p-toluenesulfonyl hydrazide, both in a similar manner as described in the examples above to provide the desired product: LCMS m/z 321 (M⁺+1).

### Example 60

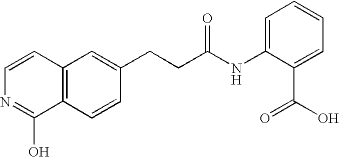

EXAMPLE 59 (170 mg, 0.5 mmol) was diluted into (1:1) methanol-methylene chloride (10 mL), treated with meta-chloroperbenzoic acid (4 equiv, 340 mg) and solid sodium bicarbonate (10 equiv, 420 mg), and the reaction mixture stirred for 5 h. The mixture was then filtered, concentrated in vacuo, and the residue purified via preparative RPHPLC to provide the isoquinoline N-oxide. This isoquinoline N-oxide (30 mg, 0.088 mmol) was diluted into toluene (15 mL), treated with acetic anhydride (3 equiv, 24 uL), and the reaction mixture was refluxed for 4 h. An additional excess of acetic anhydride (140 uL) was added, and the mixture refluxed overnight, cooled to room temperature, and then concentrated in vacuo. Purification of the residue via preparative RPHPLC with TFA-acetonitrile-water, served to hydrolyze the acetate and provide the desired hydroxyisoquinoline product: ¹H NMR (CD₃OD, 500 MHz) δ 11.41 (1H, s), 8.54 (1H, d), 8.23 (1H, d), 8.06 (1H, q), 7.56 (2H, m), 7.47 (1H, m), 7.14 (2H, t), 6.62 (1H, d), 3.20 (2H, t), 2.85 (2H, t); LCMS m/z 337 (M⁺+1).

### Example 61

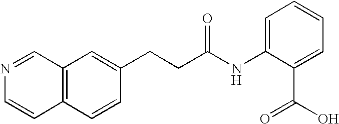

EXAMPLE 61 was prepared from commercially available 7-hydroxyisoquinoline by triflation and Heck coupling in a similar manner as described in EXAMPLE 54 above and illustrated in Scheme 9. The resultant isoquinoline acrylamide benzyl ester intermediate was hydrogenated with catalytic palladium on carbon in ethyl acetate in a similar manner as described in the examples above to provide the desired product: ¹H NMR (CDCl₃, 500 MHz) δ 11.2 (s, 1H), 10.1 (s, 1H), 8.6 (s, 1H), 8.4 (d, 1H), 8.3 (d, 1H), 8.15 (d, 1H), 8.1 (d, 1H), 8.0 (q, 1H), 7.5 (m, 1H), 7.1 (m, 1H), 3.5 (t, 2H), 3.1 (t, 2H); LCMS m/z 320 (M⁺).

### Example 62

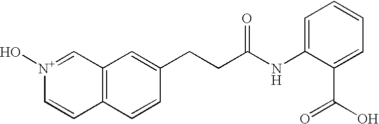

The isoquinoline acrylamide benzyl ester intermediate from EXAMPLE 61 was reduced with p-toluenesulfonyl hydrazide and oxidized with meta-chloroperbenzoic acid, both in a similar manner as described in the examples above. This saturated isoquinoline N-oxide benzyl ester was saponified with LiOH in a similar manner as described in the examples above to provide the desired product upon purification by RPHPLC: ¹H NMR (CDCl₃, 500 MHz) δ 11.0 (s, 1H), 9.3 (s, 1H), 8.5 (d, 1H), 8.3 (d, 1H), 8.0 (m, 2H), 7.9 (d, 1H), 7.8 (d, 1H), 7.7 (d, 1H), 7.5 (t, 1H), 7.1 (t, 1H), 3.3 (t, 2H), 2.9 (t, 2H).

### Example 63

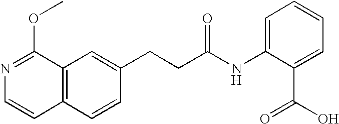

Commercially available 7-hydroxyisoquinoline (1 g, 6.9 mmol) was combined with triisopropylsilyl trifluoromethanesulfonate (3.7 mL, 13.8 mmol) in (1:1) pyridine-dimethylformamide (10 mL). Upon reaction completion, the mixture was partitioned between saturated aqueous copper sulfate and ethyl acetate, the organic phase separated, dried, and concentrated in vacuo. The crude silyl ether was purified by flash column chromatography (Biotage, SiO₂, 30% acetone-hexane) to provide the pure product (2.4 g) which was oxidized with meta-chloroperbenzoic acid in a similar manner as described in the examples above. This TIPS-ether isoquinoline N-oxide (1.95 g, 6.14 mmol) was combined with toluenesulfonyl chloride (1.5 g, 7.9 mmol), triethylamine (1.7 mL, 12.3 mmol), and maintained overnight in methanol (30 mL). The crude methoxyisoquinoline product was purified by flash column chromatography (Biotage, SiO₂), and then desilylated with HF-pyridine in tetrahydrofuran. Triflation of the phenolic moiety and Heck coupling was performed in a similar manner as described in EXAMPLE 54 above and illustrated in Scheme 9. The resultant methoxyisoquinoline acrylamide benzyl ester intermediate was hydrogenated with Pearlman's catalyst in a similar manner as described in the examples above to provide the desired product: ¹H NMR (CD₃OD 500 MHz) δ 8.6 (d, 1H), 8.1 (s, 1H), 8.0 (d, 1H), 7.9 (d, 1H), 7.8 (d, 1H), 7.7 (d, 1H), 7.5 (m, 1H), 7.3 (d, 1H), 7.1 (t, 1H), 4.1 (s, 3H), 3.2 (t, 2H), 2.8 (t, 2H); LCMS m/z 351 (M⁺+1).

### Example 64

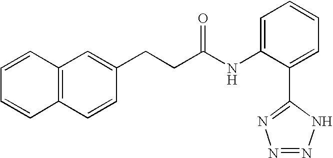

3(2-Naphthyl)propionic acid (100 mg, 0.50 mmol), prepared by hydrogenation of commercially available 3(2-naphthyl)acrylic acid, was coupled with commercially available anthranilonitrile in a similar manner as described in EXAMPLE 1 and illustrated in Scheme 1. The resultant cyanoanilide (50 mg, 0.17 mmol) was diluted into toluene (3 mL), treated with trimethylsilylazide (70 uL), followed by dibutyltin oxide (20 mg), and the reaction mixture was refluxed overnight, becoming homogeneous. The mixture was concentrated in vacuo, and the residue was purified by RPHPLC to provide the desired tetrazole product: ¹H NMR (acetone-d₆, 500 MHz) δ 8.75 (d, 1H), 8.03 (d, 1H), 7.83 (m, 5H), 7.56 (t, 1H), 7.52 (d, 1H), 7.44 (m, 3H), 7.23 (t, 1H), 3.28 (t, 2H), 2.97 (t, 2H); LCMS m/z 342 (M⁺−1).

### Example 65

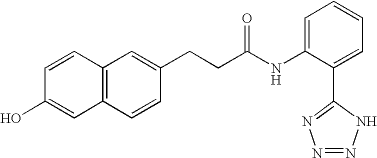

EXAMPLE 65 was prepared from commercially available 6-bromo-2-naphthol in a manner similar to EXAMPLE 17 and illustrated in Scheme 5 with the substitution of the acrylamide benzyl ester with an acrylamide nitrile. The resultant naphthol acrylamide nitrile was hydrogenated with Pearlman's catalyst in a manner similar to the examples above. As in EXAMPLE 64 above, this saturated propanamide intermediate nitrile (4 mg, 0.01 mmol) was diluted into toluene (1 mL), treated with trimethylsilylazide (10 uL, 0.04 mmol), followed by dibutyltin oxide (0.5 mg, 0.002 mmol), and the reaction mixture was refluxed 30 h. The mixture was concentrated in vacuo, and the residue was purified by RPHPLC to provide the desired tetrazole product: ¹H NMR (CD₃OD, 500 MHz) δ 8.26 (d, 1H), 7.76 (d, 1H), 7.50-7.43 (m 4H), 7.21-7.19 (m, 2H), 6.94-6.91 (m, 2H), 3.08 (t, 2H), 2.74 (t, 2H); LCMS m/z 360 (M⁺+1).

### Example 66

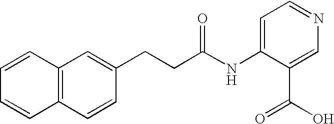

Commercially available 4-chloronicotinic acid (1 g, 6.36 mmol) was combined with 30% ammonia in water (20 mL) in an autoclave, and the reaction mixture was heated at 180° C. for 6 h. The mixture was cooled to room temperature, concentrated until a light yellow solid precipitated from solution, and then the 4-aminonicotinic acid product was filtered pure. 3(2-Naphthyl)propionic acid (20 mg, 0.10 mmol), prepared by hydrogenation of commercially available 3(2-naphthyl)acrylic acid, was diluted into toluene (2 mL) and treated with thionyl chloride (0.2 mL). The reaction mixture was heated at reflux for 2 h, cooled to room temperature, and concentrated in vacuo several times from toluene (azeotrope water). The residue was re-dissolved in toluene (2 mL), and treated with the 4-aminonicotinic acid intermediate (14 mg, 1.0 mmol). The reaction mixture was refluxed for 2 h, cooled to room temperature, and concentrated in vacuo. The residue was purified by RPHPLC to provide the desired product: ¹H NMR (DMSO-d₆, 500 MHz) δ 9.07 (s, 1H), 8.64 (dd, 2H), 7.85 (q, 2H), 7.76 (s, 1H), 7.46 (m, 4H), 3.13 (t, 2H), 2.97 (t, 2H); LCMS m/z 321 (M⁺+1).

### Example 67

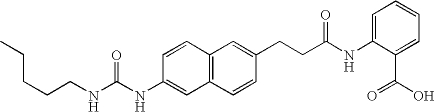

EXAMPLE 67 was prepared in a similar manner as EXAMPLE 44 with the use of pentyl isocyanate. The desired product was characterized by the following data: ¹H NMR (DMSO-d₆, 500 MHz) δ 10.16 (s, 1H), 7.62 (s, 1H), 7.48 (d, 1H), 6.97 (d, 1H), 6.70-6.56 (m, 4H), 6.41-6.35 (m, 2H), 6.14 (t, 1H), 5.25 (s, 1H), 2.11-2.06 (m, 4H), 1.80 (t, 2H), 0.45 (t, 3H), 0.3 (br s, 4H), 0.11 (t, 2H); LCMS m/z 448 (M⁺+1).

### Example 68

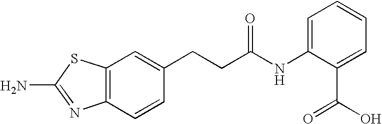

To EXAMPLE 53 (6 mg) was added 1 mL of neat trifluoroacetic acid at 0° C. The solution was warmed to room temperature, stirred for 2 hours and then stored at 0° C. for 2 days. The dark solution was then diluted with acetonitrile and purified by RPHPLC (Gilson) to give the desired product as a white solid. ¹H NMR (acetone-d₆, 500 MHz) δ 8.52 (1H, d), 8.05 (1H, d), 7.69 (1H, s), 7.54 (1H, t), 7.41 (2H, s), 7.14 (1H, t), 3.13 (2H, t), 2.79 (2H, t); LCMS m/z 342 (M⁺+1).

### Example 69

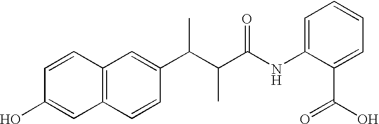

Commercially available 2-bromo-6-methoxynaphthalene (2.7 g, 11.5 mmol) in anhydrous tetrahydrofuran (20 mL) was chilled to −78° C. under nitrogen, and treated dropwise with a solution of tert-butyllithium (1.7 M, 14.2 mL, 24.2 mmol). The reaction mixture was aged for 1 h, and then treated with CuI (2.2 g, 11.5), aged 30 min, and then treated with a solution of 2-butenoic acid (500 mg, 5.8 mmol) in 30 mL of anhydrous tetrahydrofuran under nitrogen atmosphere. The reaction mixture was aged for 30 minutes at −78° C., treated with 2 equivalents of methyl iodide (neutralized through basic alumina), the mixture aged for 30 minutes and warmed to room temperature. The mixture was partitioned between 1N NaOH and ethyl acetate, the aqueous phase acidified with 2N HCl to pH 2, washed with ethyl acetate, the organic phase was separated and dried over anhydrous sodium sulfate, and then evaporated under reduced pressure to provide the crude acid product (500 mg) which is defined as Compound G in Scheme 3. Compound G was converted into EXAMPLE 69 in a manner similar to EXAMPLE 1 and illustrated in Scheme 1 using anthranilic acid directly in the amide coupling reaction, followed by demethylation of the methyl ether under conditions described for EXAMPLE 6. The desired product was characterized by the following data: ¹H NMR (CD₃OD, 500 MHz) δ 8.61 (d, 1H), 8.05 (d, 1H), 7.68 (d, 1H), 7.61 (d, 1H), 7.59 (d, 1H), 7.57 (s, 1H), 7.29 (d, 1H), 7.18 (t 1H), 7.10 (s 1H), 7.06 (m, 1H), 3.05 (m, 1H), 2.65 (m, 1H), 1.36 (d, 3H), 1.02 (d, 3H); LCMS m/z 362 (M⁺−1).

### Example 70

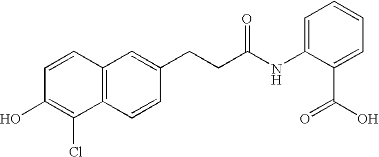

EXAMPLE 70 was prepared in a similar manner as EXAMPLE 19, except that the acrylamide methyl ester was used in the Heck coupling. The resultant double bond was hydrogenated with Pearlman's catalyst, and the methyl ester was saponified with lithium hydroxide as described before. The synthesis of the required 6-bromo-1-chloro-2-hydroxynaphthalene starting material has been described in the literature: Vyas, P. V.; Bhatt, A. K.; Ramachandraiah, G.; Bedekar, A. V. Tetrahedron Letters 2003, 44(21), 4085-4088. EXAMPLE 70 was characterized by the following data: ¹H NMR (DMSO-d₆, 500 MHz) δ 10.29 (s, 1H), 9.47 (s, 1H), 7.61 (d, 1H), 7.09 (t, 2H), 6.87-6.82 (m, 2H), 6.72 (t, 1H), 6.67 (d, 1H), 6.39 (d, 1H), 6.28 (t, 1H), 2.23 (t, 2H), 1.95 (t, 2H); LCMS m/z 370 (M⁺+1).

### Example 71

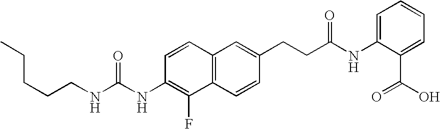

Commercially available 6-bromo-2-aminonaphthalene (100 mg, 0.45 mmol) was dissolved in 3 mL of acetonitrile, and ACCUFLUOR (160 mg, 0.495 mmol) was added. The resulting reaction mixture was stirred at room temperature before being filted through Celite and concentrated under reduced pressure. Purification of the crude product (PTLC, SiO₂) provided 6-bromo-2-amino-1-fluoronaphthalene (90 mg). This intermediate was elaborated into EXAMPLE 71 under similar conditions described for EXAMPLE 44 with the use of pentyl isocyanate. The desired product was characterized by the following data: ¹H NMR (CD₃OD, 500 MHz) δ 8.43 (d, 1H), 7.97-7.93 (m, 2H), 7.79 (d, 1H), 7.59 (s, 1H), 7.43 (d, 1H), 7.39-7.34 (m, 2H), 7.01 (d, 1H), 3.13 (t, 1H), 3.09-3.05 (m, 3H), 1.49-1.39 (m, 2H), 1.30-1.21 (m, 4H), 0.85 (t, 2H); LCMS m/z 466 (M⁺+1).

### Example 72

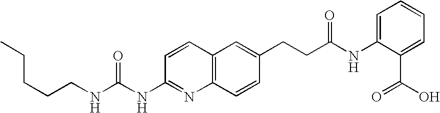

As shown in Scheme 10, commercially available 2,6-dihydroxyquinoline (100 mg, 0.62 mmol) was diluted into (1:1) pyridine-DMF (4 mL), treated with triisopropylsilyl triflate (183 uL, 0.68 mmol) and heated at 70° C. The reaction mixture was cooled to room temperature, partitioned between saturated aqueous copper sulfate and ethyl acetate, the organic phase separated, dried, concentrated in vacuo, and the crude purified via preparative RPHPLC. This silyl ether intermediate (75 mg, 0.24 mmol) was diluted into methylene chloride (4 mL), and then treated with triethylamine (98 uL, 0.71 mmol), and trifluoromethanesulfonic anhydride (44 uL, 0.26 mmol). Upon reaction completion, the reaction mixture was concentrated in vacuo, and the triflate was purified on preparative RPHPLC. This triflate intermediate was scaled up (1.9 g, 4.23 mmol), diluted into benzyl alcohol (10 mL), with DMF (30 mL), and then treated with DPPF ligand (117 mg, 0.21 mmol) and palladium acetate (285 mg, 0.42 mmol). The reaction mixture was heated at 60° C. for 3 h under 1 atmosphere of carbon monoxide gas (balloon). The mixture was cooled to room temperature, filtered through celite, washed with ethyl acetate, and the eluent concentrated in vacuo. The residue was then partitioned between water and EtOAc to remove DMF, the organic extracts separated and reduced in volume, and then subjected to distillation to remove remaining benzyl alcohol. The black residue was purified (SiO₂), and then treated with 1 equivalent of tetrabutylammonium fluoride in THF. The reaction mixture was aged for 30 min, partitioned between water and methylene chloride, and the organic extracts separated and concentrated in vacuo. The crude phenol was purified (SiO₂) (200 mg, 0.72 mmol), and converted to its triflate as described above. This crude dried triflate was not purified, but combined with the acrylamide methyl ester (190 mg, 0.93 mmol) described in EXAMPLE 53, along with triethylamine (109 uL, 0.79 mmol), palladium acetate (12 mg, 2.5%), DPPP (8 mg, 0.019 mmol), and diluted into dry degassed DMF (10 mL). The reaction mixture was heated to 80° C. overnight in a sealed tube, cooled to room temperature, filtered, partitioned between water and ethyl acetate, and the organic phase separated, dried, and concentrated in vacuo. The residue was purified via preparative RPHPLC. This acrylamide intermediate (333 mg, 0.71 mmol) was hydrogenated (balloon) over Pearlman's catalyst in (1:1) methanol-methylene chloride (10 mL). The reaction mixture was filtered (C18 SiO₂ plug), washed with acetonitrile (0.05% TFA), concentrated in vacuo, and the product purified via preparative RPHPLC. This quinoline acid (30 mg, 0.079 mmol) was diluted into chloroform (2 mL), and treated with triethylamine (32 uL, 0.24 mmol) and diphenylphosphoryl azide (102 uL, 0.48 mmol). The reaction mixture was heated at 80° C., cooled to room temperature, concentrated in vacuo, and the residue purified via preparative RPHPLC. The resultant aminoquinoline (3 mg) was diluted into methylene chloride (2 mL), treated with pentyl isocyanate (30 uL), and the mixture warmed at 40° C. overnight. The mixture was concentrated in vacuo, and the residue purified via preparative RPHPLC. The penultimate methyl ester intermediate was then saponified with LiOH in (3:1:1) THF-MeOH—H₂O in a similar manner as described in the examples above, and the acid was purified via preparative RPHPLC to provide the desired product: ¹H NMR (CD₃OD, 500 MHz) δ 8.6 (d, 1H), 8.1 (d, 1H), 7.9 (s, 1H), 7.7 (t, 2H), 7.6 (s, 1H), 7.5 (t, 1H), 7.3 (t, 2H), 7.1 (t, 1H), 3.1 (m, 5H), 2.8 (t, 2H), 1.6 (m, 2H), 1.4 (m, 5H), 1.0 (t, 3H); LCMS m/z 448 (M⁺).

### Example 73

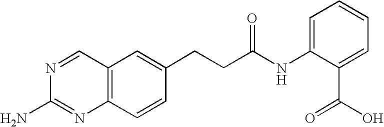

As shown in Scheme 14, guanidine carbonate (5.4 g, 0.03 mol) was added to the DMA (75 mL) solution of 3-bromo-6-fluoro-benzaldehyde (4.06 g, 0.02 mol) at room temperature. The solution was heated to 140° C. overnight, and the solvent was removed in vacuo. The residue was worked up with AcOEt/H₂O. The organic layer was dried, and the residue was recrystallized with CH₂Cl₂/MeOH to obtain 6-bromo-2-quinazolinamine. To this bromide intermediate (100 mg, 0.448 mmol), Pd(OAc)₂ (10 mg) and P(O-tol)₃ (29 mg) were added to a Et₃N (2 mL) solution of the acrylamide (252 mg, 0.896 mmol) under nitrogen. The solution was degassed for 5 min and heated to 100° C. for 12 h. The reaction mixture was diluted with 20 mL AcOEt, filtered, washed with H₂O and dried in vacuo. The residue was purified by RPHPLC. This intermediate (70 mg) in a solution of MeOH, a few drops of CH₂Cl₂, two drops of TFA and 10 mg Pd(OH)₂ was hydrogenated for 16 h at room temperature. The product was obtained after filtration and dried in vacuo. A water (2 mL) solution of ceric ammonium nitrate (195 mg) was added to this intermediate (59 mg) in acetone (2 mL) at room temperature and stirred for 2 h. The solution was diluted with AcOEt (10 mL) and washed with water (5 mL). The organic layer was dried and purified by RPHPLC to obtain the desired product. ¹H NMR (DMSO-d₆, 500 MHz) δ 8.99 (s, 1H), 8.50 (d, 1H), 7.58 (m, 3H), 7.32 (d, 1H), 7.18 (d, 1H), 6.73 (s, 1H), 2.91 (t, 2H), 2.74 (t, 2H); LCMS m/z 337 (M⁺+1).

### Example 74

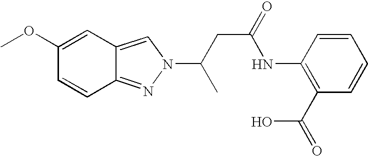

As shown in Scheme 15, acetylchloride (2.78 mL, 38.1 mmol, 1.05 eq) was added to a THF (200 mL) solution of 2-methyl-4-methoxylaniline (5 g, 36.3 mmol, 1 eq) and Et₃N (6.31 mL, 45.4 mmol, 1.25 eq.) at 0° C. in 5 min. The solution was warmed up to room temperature for 4 h and filtered through a silica gel pad. The crude product was obtained after removing the solvent in vacuo. Isoamylnitrite (4.54 g, 55.85 mmol, 2.9 eq) was added to a chloroform (100 mL) solution of this crude intermediate acetamide (3.45 g, 19.27 mmol, 1 eq), KOAc (3.78 g, 38.54 mmol, 2 eq), HOAc (2.31 g, 38.54 mmol, 2 eq), Ac₂O (3.94 g, 38.54 mmol, 2 eq) and 18-crown-6 (1.01 g, 3.65 mmol, 0.2 eq) at RT. The solution was heated to reflux overnight, followed by washing with H₂O, NaHCO₃ and brine, and then chromatographed on SiO₂ with EtOAc/Hexanes (1:4) to obtain the desired product. Solid t-BuOK (0.725 g g, 3.72 mmol, 1.1 eq) was added to a DMF (10 mL) solution of the indazole intermediate (0.5 g, 3.38 mmol, 1 eq), and the mixture stirred for 30 min at 0° C. Ethyl-3-bromo-butanoate was added and the solution was warmed to RT for 3 h. To this solution, 1N NaOH (7 mL) was added and stirred for another 2h. The reaction solution was washed with Et₂O (2×10 mL), then acidified with 3N HCl to pH=7 and extracted with EtOAc (2×20 mL). The organic extracts were purified on RPHPLC to obtain two N-alkyl indazole regioisomeric fractions. The desired EXAMPLE 74 was then obtained using similar procedures as described above. ¹H NMR (CD₃OD, 500 MHz) δ 8.44 (d, 1H), 8.16 (s, 1H), 8.01 (dd, 1H), 7.51 (m, 2H), 7.13 (t, 1H), 6.98 (m, 2H), 5.22 (m, 1H), 3.80 (s, 3H), 3.23 (m, 1H), 3.07 (m, 1H), 1.73 (d, 3H); LCMS m/z 354 (M⁺+1).

### Example 75

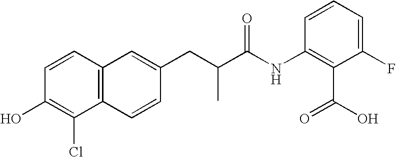

As shown in Scheme 16, a xylenes solution of 6-methoxy-2-naphthaldehyde (0.855 g, 4.585 mmol) was treated with the stabilized ylide (2.16 g, 5.96 mmol, 1.3 eq.) at room temperature. The solution was heated to reflux for 4 h. The solvent was removed under vacuum, and the residue was chromatographed with AcOEt/Hexanes (4:1) to obtain the product. To a methanol solution of the enoate intermediate (5.73 g) was added Pd/C (0.3 g), and the mixture was hydrogenated under a balloon at room temperature overnight. The solution was filtered, and the solvent was removed in vacuo to obtain the product. Then N-chlorosuccinimide (0.82 g, 6.11 mmol, 1.1 eq) was added to a DMF solution of this intermediate at room temperature, and the solution was stirred overnight. The DMF was removed in vacuo, and the residue was recrystallized with methanol/dichloromethane to obtain the desired product, utilized for enantiomeric resolution below.

### Chiral Resolution of EXAMPLE 75 Intermediate as its Ethyl Ester

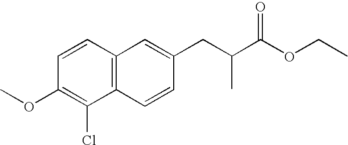

The racemic ethyl ester intermediate of EXAMPLE 75 was resolved into its enantiomers: Preparative ChiralCell OJ, 35% isopropanol-heptane; isocratic elution. These enantiomeric intermediates (65 mg, 0.21 mmol) were dissolved in AcOH/HCl (1:1, 2 mL), and heated to 110° C. for 10 min. Then (5 mL) of water was added, the solution cooled to 0° C., and the acid product was obtained after filtration. These enantiomeric acid intermediates were used to acylate a fluoro anthranilic acid derivative, using similar procedures as described above, to obtain the desired EXAMPLE 75 in both enantiomeric forms. ¹H NMR (CDCl₃, 500 MHz) δ 8.36 (d, 1H), 7.98 (d, 1H), 7.57 (m, 2H), 7.41 (m, 2H), 7.17 (d, 1H), 6.81 (dd, 1H), 3.23 (m, 1H), 2.87 (m, 1H), 2.78 (m, 1H), 1.27 (d, 3H); ¹H NMR (CD₃OD, 500 MHz) δ 7.00 (d, 1H), 7.86 (d, 1H), 7.58 (m, 2H), 7.41 (m, 2H), 7.14 (d, 1H), 6.90 (m, 1H), 3.15 (m, 1H), 2.86 (m, 2H), 1.25 (d, 3H); LCMS m/z 400 (M⁺−1).

### Examples 76-80

The two enantiomeric acid intermediates from EXAMPLE 75 above, were used to acylate a variety of fluorinated anthranilic acid derivatives, including an aminopyridine. The following examples were prepared using similar procedures as described above.

| EXAMPLE | | LCMS (m/z) |
|---|---|---|
| 76 | 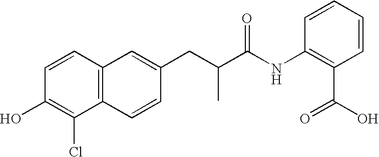 | 382 (M⁺ − 1) |
| 77 | 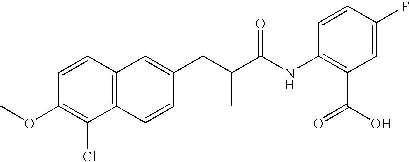 | 414 (M⁺ − 1) |
| 78 | 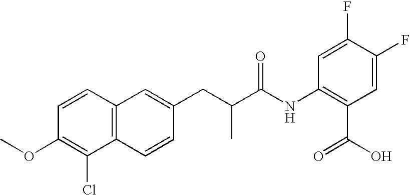 | 432 (M⁺ − 1) |
| 79 | 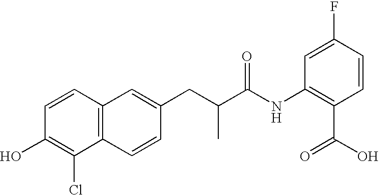 | 400 (M⁺ − 1) |
| 80 | 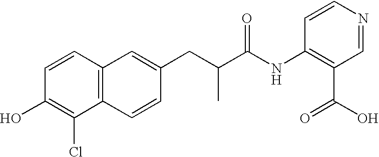 | 385 (M⁺ + 1) |

NMR data for selected Examples:

### Example 76

¹H NMR (CDCl₃, 500 MHz) δ 10.85 (s, 1H), 8.77 (d, 1H), 8.07 (d, 1H), 7.99 (d, 1H), 7.63 (m, 2H), 7.48 (d, 2H), 7.22 (d, 1H), 7.16 (t, 1H), 3.30 (m, 1H), 2.96 (m, 1H), 2.86 (m, 1H), 1.36 (d, 3H).

### Example 77

¹H NMR (CDCl₃, 500 MHz) δ 11.15 (s, 1H), 8.67 (m, 1H), 8.10 (d, 1H), 7.70 (m, 2H), 7.59 (s, 1H), 7.43 (d, 1H), 7.23 (m, 2H), 4.00 (s, 3H), 3.24 (m, 1H), 2.85 (m, 1H), 2.80 (m, 1H), 1.28 (d, 3H).

### Example 78

¹H NMR (CDCl₃, 500 MHz) δ 11.33 (s, 1H), 8.61 (m, 1H), 8.07 (d, 1H), 7.80 (t, 1H), 7.65 (d, 1H), 7.56 (s, 1H), 7.39 (d, 1H), 7.24 (d, 1H), 3.99 (s, 3H), 3.22 (m, 1H), 2.86 (m, 1H), 2.78 (m, 1H), 1.26 (d, 3H).

### Example 79

¹H NMR (CD₃OD, 500 MHz) δ 8.38 (dd, 1H), 8.05 (m, 1H), 7.98 (d, 1H), 7.56 (m, 2H), 7.40 (m, 1H), 7.12 (d, 1H), 6.82 (t, 1H), 3.15 (m, 1H), 2.91 (m, 1H), 2.85 (m, 1H), 1.28 (d, 3H).

### Example 80

¹H NMR (DMSO-d₆, 500 MHz) δ 10.31 (s, 1H), 8.97 (s, 1H), 8.56 (d, 1H), 8.45 (d, 1H), 7.90 (d, 1H), 7.66 (s, 1H), 7.65 (d, 1H), 7.46 (dd, 1H), 7.22 (d, 1H), 3.22 (m, 1H), 2.90 (m, 1H), 2.85 (m, 1H), 1.19 (d, 3H).

### Example 81

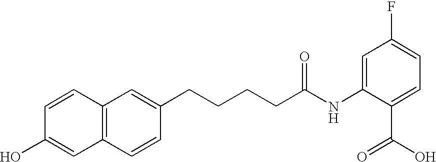

EXAMPLE 81 was prepared in a similar manner as the synthesis of EXAMPLE 14, using a fluoro anthranilic acid derivative. The desired product was purified via preparative RPHPLC. ¹H NMR (CD₃OD, 500 MHz) δ 8.41 (d, 1H), 8.18 (m, 1H), 7.62 (d, 1H), 7.55 (d, 1H), 7.52 (s, 1H), 7.25 (dd, 1H), 7.05 (m, 2H), 6.89 (t, 1H), 2.78 (t, 2H), 2.50 (t, 2H), 1.79 (m, 4H); LCMS m/z 380 (M⁺−1).

### Example 82

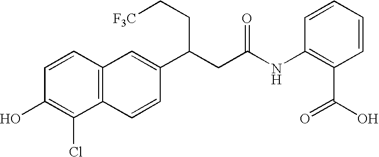

As shown in Scheme 17, 3,3,3,-trifluoropropanaldehyde (1.0 g) was dissolved in 20 mL dichloromethane and methyl (triphenylphosphoranylidene) acetate (2.7 g) was added and the resulting reaction mixture was stirred at room temperature for 15 hours before being concentrated under reduced pressure. Column chromatography (SiO₂, acetone/hexanes) gave the desired unsaturated ester product (1.78 g). This intermediate (700 mg) was dissolved in 20 mL of argon degassed triethylamine and 6-benzyloxy-2-bromo-5-chloro-naphthalene (1.5 g), palladium acetate (75 mg), phosphorus triortho toluene (40 mg) were added and the resulting reaction mixture was heated to 100° C. for 15 hours. After cooling, filtration through celite, and evaporation under reduced pressure the reaction residue was purified by column chromatography (SiO₂, ethyl acetate/hexanes) giving the desired naphthalene derived product (290 mg). This intermediate (250 mg) was dissolved in THF (5 mL), MeOH (5 mL) and 1N LiOH aq. (10 mL), and resulting reaction mixture was stirred at room temperature for 4 h. The reaction mixture was then made acidic with concentrated HCl (aq.) and extracted with ethyl acetate. Concentration of the resulting organic layers yielded the desired carboxylic acid derived product that was used without any further purification. This intermediate (88 mg) was dissolved in 3 mL of dichloromethane, cooled to 0° C. before oxayl chloride (2 M, 0.5 mL) and DMF (0.01 mL) were added. The resulting reaction mixture was heated to 40° C. for 30 min, then evaporated under reduced pressure. The residue was then taken up in THF (3 mL) and triethylamine (0.12 mL) and anthranilic acid was added before the reaction mixture was allowed to stir at room temperature for 15 hours. Extraction with ethyl acetate and subsequent concentration of the organic layers yielded a residue that was purified with preparative RPHPLC to give the desired anthranilic acid intermediate. This intermediate (20 mg) was dissolved in a dichloromethane/methanol mixture, catalytic palladium hydroxide was added, and the resulting reaction mixture was exposed to a hydrogen atmosphere for 3 h. Following filtration through celite, the concentrated residue was purified with preparative RPHLPC to yield the desired final product. ¹H NMR (CD₃OD, 600 MHz) δ 8.39 (d, 1H), 8.05 (d, 1H), 7.98 (dd, 1H), 7.66 (dd, 1H), 7.62 (d, 1H), 7.62-7.44 (m, 1H), 7.14 (d, 1H), 7.05 (t, 1H), 3.39-3.37 (m, 1H), 2.89 (dd, 1H), 2.79 (dd, 1H), 2.14-2.07 (m, 2H), 2.02-198 (m, 1H), 1.92-188 (m, 1H); LCMS m/z 466 (M⁺+1).

### Example 83

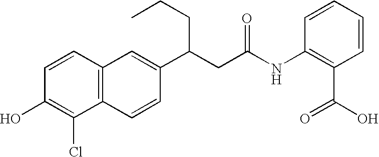

EXAMPLE 83 was prepared under similar conditions described above for EXAMPLE 82. Enantiomers were separated with a Gilson ChiralPak AD column running 15% isocratic isopropanol/heptane with 0.1% trifluoro acetic acid: Enantiomer A—retention time 31.6 min, Enantiomer B—retention time 38.45 min; ¹H NMR (CD₃OD, 600 MHz) δ 8.38 (d, 1H), 7.98 (t, 2H), 7.60 (d, 1H), 7.57 (d, 1H), 7.45-7.42 (m, 2H), 7.04 (t, 1H), 3.34 (m, 1H), 2.81 (dd, 1H), 2.71 (dd, 1H), 1.74 (q, 2H), 1.25-1.16 (m, 2H), 0.86 (t, 3H); LCMS m/z 412 (M⁺+1).

### Example 84

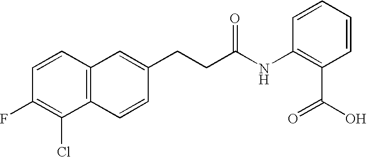

6-amino-2-bromo-naphthalene (500 mg) was dissolved in 15 mL of DMF, cooled to 0° C., and N-chlorosuccinamide (300 mg) was added and the reaction was warmed to room temperature over 3 h. The reaction mixture was then extracted with water and dichloromethane, and the resulting organic layers were evaporated under reduced pressure to yield 6-amino-5-chloro-2-bromo-naphthalene, following purification on silica gel (ethyl acetate/hexanes). 6-amino-5-chloro-2-bromo-naphthalene (1.5 g) was dissolved in HF pyridine (75 mL) and sodium nitrite (1.1 g) was added. The resulting reaction mixture was heated to 90° C. for 3 h, before cooling to room temperature, and purification on silica gel (hexanes) yielded 2-bromo-5-chloro-6-fluoro naphthalene. This intermediate was elaborated into EXAMPLE 84 according to Scheme 5, under similar conditions as in EXAMPLE 18. ¹H NMR (DMSO-d₆, 500 MHz) δ 10.29 (s, 1H), 7.57 (d, 1H), 7.18 (d, 1H), 7.06 (d, 1H), 6.95 (m, 2H), 6.73 (t, 2H), 6.70-6.64 (m, 1H), 6.55 (t, 1H), 6.22 (t, 1H), 2.24 (t, 2H), 1.94 (t, 2H).

### Example 85

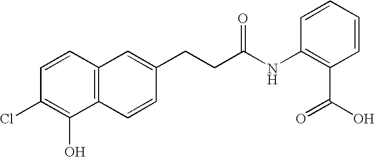

Commercially available 6-amino-1-naphthol (3 g, 0.02 mol) was dissolved in anhydrous methylene chloride under argon atmosphere at 0° C. The solution was treated with imidazole (2.56 g, 0.04 mol) and tert-butyldimethylsilyl chloride and allowed to warm to room temperature for 15 h. The reaction mixture was partitioned between water and methylene chloride, the organic phase separated, dried over anhydrous sodium sulfate, and evaporated under reduced pressure. The crude product was purified by flash column chromatography (Biotage, SiO₂, 15% Ethyl acetate/Hexane). This intermediate naphthol (1 g, 3.66 mmol) was dissolved in anhydrous acetonitrile under argon atmosphere and cooled to 0° C. To this solution was added tetrafluoroboric acid (0.7 mL, 7.32 mmol), tert-butyl nitrite (0.7 mL, 5.49 mmol), and the resulting reaction mixture was stirred at 0° C. for 30 min. A catalytic amount of palladium acetate and the acrylamide benzyl ester (2.11 g, 7.32 mmol), [which was obtained from using commercially available benzyl anthranilate and acryolyl chloride under previously described conditions in EXAMPLE 17], was dissolved in 20 mL of anhydrous methanol and added to the reaction mixture. After allowing the mixture to warm to room temperature for 1.5 h, it was partitioned between water and ethyl acetate, the organic phase separated, dried, and concentrated under reduced pressure. The crude product was first purified with a plug of SiO₂ (25% Acetone/Hexane) to remove baseline impurities followed by flash column chromatography (Biotage, SiO₂, 5%-25% Acetone/Hexane). Preparative RPHPLC removed remaining impurities to provide both the TBS protected and deprotected intermediates. A fraction of the deprotected intermediate (41 mg, 0.10 mmol) was combined with DMF (2 mL) and N-chlorosuccinimide (26 mg, 0.01 mmol) in a sealed tube and heated to 55° C. and monitored by TLC. After 20 min, the reaction mixture was partitioned between water and ethyl acetate, the organic phase separated, dried, and concentrated under reduced pressure. The crude product was purified by preparative RPHPLC. This intermediate was dissolved in methanol (1.5 mL) and methylene chloride (1.5 mL), treated with catalytic palladium hydroxide, then exposed to hydrogen at 1 atmosphere for 15 min. Following filtration through celite, the concentrated residue was purified with preparative RPHLPC to yield the desired final product. ¹H NMR (DMSO-d₆, 500 MHz) δ10.31 (d, 1H), 9.54 (s, 1H), 9.08 (s, 1H), 7.61 (d, 1H), 7.26 (d, 1H), 7.10 (d, 1H), 7.05-6.87 (d, 1H), 6.72 (t, 1H), 6.65 (q, 1H), 6.57-6.50 (m, 2H), 6.28 (t, 1H), 2.31 (t, 1H), 2.25 (t, 1H), 1.97 (q, 2H); LCMS m/z 370 (M⁺+1).

### Example 86

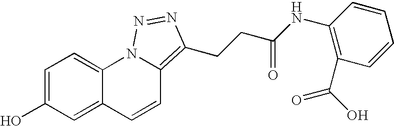

To a solution of diisopropylamine (2.34 g, 3.3 mL, 23 mmol) in 50 mL of THF was added n-butyllithium (16 mL, 25.3 mmol, 1.6 M in hexane) at −78° C. After 10 min, the resulting solution was warmed to 0° C. and stirred for 30 min. To this solution at −78° C. was added a solution of methoxyquinoline (2 g, 11.5 mmol) in 25 mL of THF dropwise. After 5 min, to this solution was added N,N,N′,N′-tetramethylethylene diamine (2.67 g, 3.5 mL, 23 mmol). The resulting red solution was stirred at −78° C. for 1 h. To this solution was then slowly added methyl chloroformate (2.17 g, 1.77 mL, 23 mol). The resulting solution was slowly warmed to RT. The solution was then quenched with water (250 mL). The mixture was then extracted with ethyl acetate (100 mL). The organic layer was dried and concentrated. The residue was purified by Biotage (2-25% ethyl acetate in hexane) to give a mixture of products, which was further purified by RP-HPLC to give the desired intermediate as a brown solid. To this intermediate (1.1 g, 4.76 mmol, washed with sodium carbonate) and sodium hydride (230 mg, 5.71 mmol, 60% in petroleum oil) was added 80 mL of THF at −78° C. The mixture was slowly warmed to RT. After 30 min, to this mixture was added 4-acetamidobenzenesulfonyl azide (1.37 g, 5.71 mmol) in one portion. The slurry was stirred at RT for 3 h. To this mixture was added water and the resulting mixture was extracted with dichloromethane (100 mL×5). The combined organic layer was dried and concentrated. The residue was taken up with methanol and filtered. The solid was washed with methanol and became light yellow. The filtrate was concentrated and purified by RP-HPLC to give the annulated triazole, which was combined with the collected light yellow solid. To a solution of this ester (300 mg, 1.17 mmol) in 20 mL of dichloromethane was added DIBALH (3.5 mL, 3.5 mmol, 1 M in toluene) at 0° C. The mixture was warmed to RT and stirred for 4 h. The mixture was then quenched with water and saturated Rochelle's salt (100 mL). The aqueous layer was then extracted with 30% isopropyl alcohol in chloroform. The combined fractions were dried with sodium sulfate and concentrated in vacuo to give the desired alcohol as a light yellow solid which contained some inorganic salt. To a solution of this alcohol in 30 mL of dichloromethane were added diacetoxy iodobenzene (450 mg, 1.4 mmol) and 15 mg of TEMPO. The resulting slurry turned clear. After 16 h at RT, the mixture was washed with sodium sulfite solution and extracted twice with 30% isopropanol in chloroform (100 mL). The combined organic fractions were dried with sodium sulfate and concentrated in vacuo to give the aldehyde as a yellow solid. To a solution of trimethylphosphonoacetate (499 mg, 0.44 mL, 2.74 mmol) in 30 mL of THF was added n-butyllithium (1.21 mL, 2.5 M in hexane, 3.0 mmol) at 0° C. After 15 min, the mixture was warmed to RT and transferred to a solution of the aldehyde (310 mg, 1.37 mmol) in 10 mL of THF. The resulting slurry was stirred at RT for 2 h and to this mixture was added 50 mL of water. The mixture was then extracted with 100 mL of ethyl acetate and 100 mL of 30% isopropanol in chloroform. The combined organic fractions were dried with sodium sulfate and concentrated to give the enoate as a yellow solid. To this enoate were added 50 mL of THF:methanol:water (3:1:1, 50 mL) and 1 N lithium hydroxide solution (15 mL). After 4 h, the clear yellow solution was washed with ethyl acetate (100 mL). The aqueous layer was acidified with concentrated HCl until precipitate appeared. This mixture was extracted four times with 30% isopropanol in chloroform (50 mL). The combined organic layers were dried with sodium sulfate and concentrated in vacuo to give the enoic acid as a yellow solid. To this acid (130 mg, 0.48 mmol) was added 3 mL of thionyl chloride. The resulting clear solution was heated at 50° C. for 30 min and thionyl chloride was removed in vacuo. To the residue was added toluene (20 mL) and then anthranilic acid (137 mg, 1.0 mmol). The mixture was heated at 120° C. for 3 h. The resulting yellow slurry was washed with acetone and methanol and filtered to give the product as a yellow solid. To a slurry of this intermediate (155 mg, 0.40 mmol) in 20 mL of methanol was added 50 mg of Pd/C 10%). The mixture was held under 45 psi of hydrogen gas overnight. The slurry was filtered and the solid was washed with acetone (50 mL) and 30% isopropanol in chloroform (500 mL). The filtrate was concentrated to give a yellow solid. To this methyl ether (40 mg, 0.10 mmol) in 5 mL of dichloromethane was added borontribromide (3 mL, 3 mmol, 1 M in dichloromethane) at 0° C. The mixture was warmed to RT and stirred for 12 h. The mixture was then quenched with water at −78° C. and warmed to RT. The mixture was concentrated and purified by RP-HPLC to give the desired compound as a white solid. ¹H NMR (CD₃OD, 500 MHz) δ 8.53 (2H, t), 8.03 (1H, dd), 7.66 (1H, d), 7.54 (1H, m), 7.50 (1H, d), 7.30 (1H, dd), 7.26 (1H, d), 7.13 (1H, t), 3.44 (2H, t), 2.99 (2H, t); LCMS m/z 377 (M⁺+1).

### Example 87

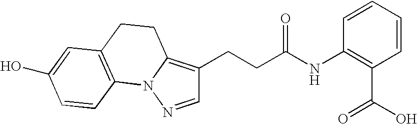

To a solution of diisopropylamine (1.3 g, 1.8 mL, 13 mmol) in 20 mL of THF was added n-butyllithium (5.5 mL, 13.8 mmol, 2.5 M in hexane) at 0° C. After 30 min, the resulting solution was cooled to −78° C. To this solution at −78° C. was added a solution of methyl acetoacetate (0.58 g, 0.54 mL, 5 mmol) in 5 mL of THF dropwise. After 30 min, to this solution was added N,N N′,N′-tetramethylethylene diamine (0.58 g, 0.75 mL, 5 mmol). The resulting red solution was warmed to 0° C. and stirred for 0.5 h. To this solution was then slowly added the benzyl bromide starting material shown in Scheme 19 (1.4 g, 5 mmol). The resulting solution was slowly warmed to RT and stirred for 2 h. The solution was then quenched with 1 N HCl (15 mL). The mixture was then extracted with ethyl acetate (100 mL). The organic layer was dried with sodium sulfate and concentrated. The residue was purified by Biotage (2-20% ethyl acetate in hexane) to provide a light yellow oil. A mixture of this intermediate ketoester (200 mg, 0.63 mmol), acetic anhydride (130 mg, 0.12 mL, 1.27 mmol) and triethyl orthoformate (93 mg, 0.11 mL, 0.63 mmol) was heated at 135° C. for 1.5 h. The crude was quickly chromatographed using 5-20% ethyl acetate in hexane to give a dark oil, which was then added to a mixture of hydrazine monohydrate (2 mL, 64-65%) and ethanol (30 mL). The mixture was heated overnight and concentrated, purified by Gilson to provide an off-white solid. A mixture of this pyrazole intermediate (30 mg, 0.088 mmol), copper (I) iodide (1 mg, 0.0044 mmol), N,N′-dimethyl ethylene diamine (1.6 mg), potassium carbonate (26 mg, 0.18 mmol) and toluene (2 mL) was heated at 110° C. under nitrogen overnight. The mixture was purified by Gilson to give a white solid. Following a similar sequence as described for the preparation of EXAMPLE 86 then provided the desired compound as a white solid. ¹H NMR (d₆-acetone, 500 MHz) δ 11.2 (1H, d), 8.75 (1H, d), 8.08 (1H, dd), 7.61 (2H, m), 7.45 (1H, s), 7.13 (1H, t), 6.76 (2H, m), 2.88 (2H, t), 2.70 (2H, t); LCMS m/z 378 (M⁺+1).

### Example 88

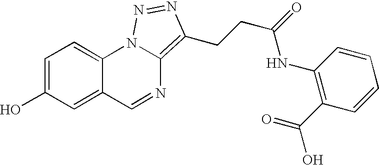

To 2-nitro-5-methoxy-benzoic acid (4 g, 20.3 mmol) in 35 mL of methanol was added trimethylsilyldiazomethane (35 mL, 70 mmol, 2 M in dichloromethane) at RT dropwise. The mixture was stirred at RT for 10 h. To the mixture was added several drops of acetic acid. The resulting solution was concentrated in vacuo to give a brown solid. To this intermediate was added 150 mg of Pd/C (10%). The mixture was stirred under 40 psi of hydrogen gas for 5 h. The mixture was filtered and washed with dichloromethane. The filtrate was concentrated in vacuo to give a dark red oil. To this aniline intermediate were added 30 mL of ethanol and 5 mL of concentrated HCl. To this mixture at 0° C. was dropwise added a solution of sodium nitrite (5.6 g, 81.2 mmol) in 15 mL of water to form the diaza salt. After 1 h at 0° C., to the resulting dark red solution was slowly added sodium azide (8.6 g, 132 mmol) in 15 mL of water. After 1 h at 0° C., the slurry was filtered and washed with saturated sodium carbonate solution and water to give the azide as a red solid. The same DIBALH reduction procedure as described above gave the benzyl alcohol as a dark red oil. To this oil in 100 mL of dichloromethane was added PCC (8 g) at 0° C. The mixture was stirred at RT for 4 h and purified by Biotage (2-20% ethyl acetate in hexane) to give the aryl azide aldehyde intermediate as a light yellow solid. To a solution of this intermediate (1.1 g, 6.3 mmol), malononitrile (423 mg, 0.40 mL, 6.4 mmol) and 15 mL of dichloromethane was added a solution of piperidine (145 mg, 0.17 mL, 1.7 mmol) in 5 mL of dichloromethane. After 2 h at RT, the mixture was filtered and the solid was washed with dichloromethane to give the tricycle as a brown solid. To this intermediate (0.66 g, 2.9 mmol) in 10 mL of DME and 20 mL of dichloromethane was added DIBALH (7.04 mL, 7.04 mmol, 1 M in hexane) at −78° C. The mixture was stirred at −78° C. for 3 days. The mixture was then quenched with water and saturated Rochelle's salt (200 mL) at −78° C. The aqueous layer was then extracted with 30% isopropyl alcohol in chloroform. The combined fractions were dried with sodium sulfate and concentrated in vacuo. The residue was purified by RP-HPLC to give a light yellow solid. A similar homologation sequence described in EXAMPLE 86 gave the intermediate enamide. To a slurry of this enamide (18 mg) in 150 mL of methanol was added p-toluenesulfonylhydrazide (400 mg). The mixture was heated at reflux overnight. After removing the solvent, the residue was purified by RP-HPLC to give a pale yellow solid. Following the similar hydrolysis and demethylation procedures as described for the preparation of EXAMPLE 86, the desired compound was obtained as a white solid. ¹H NMR (CD₃OD, 500 MHz) δ 8.50 (1H, d), 8.47 (1H, d), 8.00 (1H, d), 7.84 (1H, s), 7.52 (1H, t), 7.34 (1H, dd), 7.29 (1H, d), 7.11 (1H, t), 3.50 (2H, t), 3.06 (2H, t); LCMS m/z 378 (M⁺+1).

### Example 89

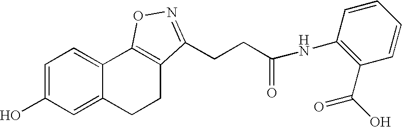

To a solution of diisopropylamine (5.3 g, 52 mmol) in 200 mL of THF was added n-butyllithium (22.4 mL, 56 mmol, 2.5 M in hexane) at −78° C. The resulting solution was stirred at −78° C. for 30 minutes and then at room temperature for an additional 30 minutes. The solution was re-cooled to −78° C. and to this solution, was added drop-wise a solution of tetralone 20 (7.03 g, 39.9 mmol) in 80 mL of THF. After 1 hour at −78° C., to the above solution was added 4-chloro-4-oxobutyrate (8.43 g, 6.84 mL, 56 mmol) in one portion. The resulting solution was warmed to room temperature over 2 hours. The solvent was then evaporated and the residue was diluted with 200 mL of THF/MeOH/water (v:v:v=3:1:1). To this mixture was added 100 mL of lithium hydroxide (1 M in water) and the resulting solution was stirred overnight. After removing some solvent in vacuo, the remaining aqueous layer was extracted with ethyl acetate (100 mL×3). The aqueous phase was acidified with HCl until pH=3. The mixture was extracted with ethyl acetate (100 mL×2). The combined organic fractions were dried with sodium sulfate and concentrated in vacuo to give the product as a grey solid. To this intermediate (81 mg) were added hydroxyamine hydrochloride (41 mg) and ethanol (20 mL). The mixture was heated at reflux overnight. After removing the solvent, to the residue was added lithium hydroxide (1 N) in THF and methanol. The mixture was stirred at RT for 5 h and concentrated. The residue was then purified by Gilson to give a mixture of two isoxazole annulated regioisomers. To one isomer (22 mg, 0.08 mmol) was added 1 mL of thionyl chloride. The resulting clear solution was heated at 75° C. for 90 min and thionyl chloride was removed in vacuo. To the residue was added toluene (10 mL) and then anthranilic acid (22 mg, 0.16 mmol). The mixture was heated at 75° C. for 2 h. The resulting yellow slurry was concentrated and purified by Gilson to give a brown solid. To this intermediate (28 mg, 0.07 mmol) in 15 mL of dichloromethane was added borontribromide (0.57 mL, 0.57 mmol, 1 M in dichloromethane) at 0° C. The mixture was warmed to RT and stirred overnight. The mixture was then quenched with water at 0° C. and warmed to RT. The mixture was concentrated and purified by RP-HPLC to give the desired compound as a white solid. ¹H NMR (CD₃OD, 500 MHz) δ 8.56 (1H, d), 8.07 (1H, dd), 7.55 (1H, m), 7.42 (1H, d), 7.14 (1H, t), 6.75 (1H, s), 6.71 (1H, dd), 3.07 (2H, t), 2.94 (2H, t), 2.87 (2H, t), 2.72 (2H, t); LCMS m/z 379 (M⁺+1).

### Example 90

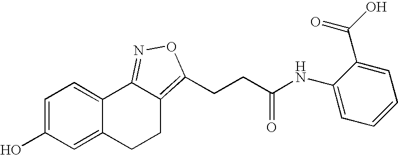

The same reaction sequence in EXAMPLE 89 above provided the regioisomer in EXAMPLE 90 as a colorless oil. ¹H NMR (CD₃OD, 500 MHz) δ 8.54 (1H, d), 8.08 (1H, dd), 7.62 (1H, d), 7.55 (1H, m), 7.14 (1H, m), 6.74 (1H, s), 6.72 (1H, m), 3.19 (2H, t), 2.87 (2H, t), 2.83 (2H, t), 2.71 (2H, t); LCMS m/z 379 (M⁺+1).

### Example 91

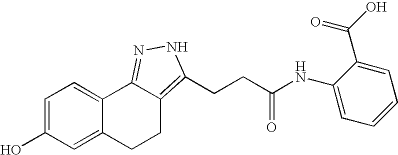

Example 91 was prepared under similar conditions described for the syntheses of EXAMPLES 89 and 90, where a hydrazine equivalent (Scheme 22) was used in place of hydroxylamine (Scheme 21). ¹H NMR (CD₃OD, 500 MHz) δ 8.54 (1H, d), 8.05 (1H, d), 7.54 (1H, t), 7.47 (1H, d), 7.13 (1H, t), 6.76 (1H, s), 6.72 (1H, dd), 3.14 (2H, t), 2.89 (4H, m), 2.76 (2H, t); LCMS m/z 378 (M⁺+1).

### Example 92

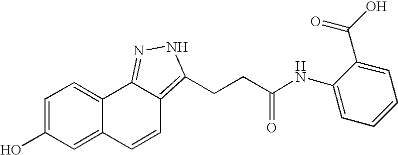

EXAMPLE 92 was isolated from EXAMPLE 91 as an over-oxidation product, upon demethylation. ¹H NMR (CD₃OD, 500 MHz) δ 8.54 (1H, d), 8.14 (1H, d), 8.03 (1H, dd), 7.64 (1H, d), 7.53 (1H, m), 7.31 (1H, d), 7.23 (1H, d), 7.13 (1H, dd), 7.11 (1H, t), 3.43 (2H, t), 2.97 (2H, t); LCMS m/z 376 (M⁺+1).

### Example 93

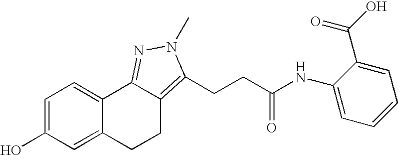

EXAMPLE 93 was prepared under similar conditions described for the syntheses of EXAMPLES 89 and 90, where a methylhydrazine equivalent (Scheme 23) was used in place of hydroxylamine (Scheme 21). The desired compound was obtained as an off-white solid. ¹H NMR (CD₃OD, 500 MHz) δ 8.50 (1H, d), 8.01 (1H, d), 7.52 (1H, t), 7.45 (1H, d), 7.11 (1H, t), 6.67 (2H, m), 3.13 (2H, t), 2.78 (4H, m), 2.67 (2H, t); LCMS m/z 392 (M⁺+1).

### Example 94

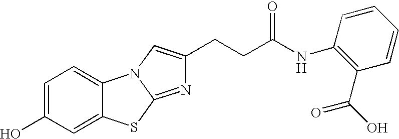

A mixture of methoxy aminobenzothiazole (8.5 g, 47 mmol.) and ethyl α-bromopyruvate (12.9 g, 59 mmol) was heated in 120 mL of DME under reflux for 2 hrs. After cooling to RT, the precipitate was collected by filtration to afford the product as a yellow solid, which was then heated in a solution of ethanol (200 mL) under reflux for 4 h. The partitioning of the resulting residue after concentration using ethyl acetate and saturated aqueous sodium carbonate solution gave an organic fraction, which was dried with sodium sulfate. The concentration in vacuo led to the tricyclic intermediate as a solid. To a solution of this ester (2.67 g, 9.65 mmol) in 100 mL of dichloromethane was added DIBALH (14.5 mL, 1 M in hexane, 14.5 mmol) at −78° C. After 1 hr at −78° C., the mixture was quenched with water and slowly warmed to RT. Saturated aqueous Rochelle's salt solution was added, and the mixture turned clear overnight. The organic phase was washed with water and concentrated. The resulting residue was filtered to give the aldehyde as a yellow solid. To a solution of trimethyl phosphonoacetate (0.71 mL, 4.33 mmol) in 40 mL of THF was added nBuLi (2.9 mL, 4.6 mmol., 1.6 M in hexane) at 0° C. After 30 min, to the solution was added the aldehyde (0.67 g, 2.88 mmol). After 10 min, the mixture was quenched with water and diluted with ethyl acetate. The organic phase was concentrated by Biotage (20-30% ethyl acetate/hexane) to give the enoate as a white solid. This enoate intermediate was transformed into EXAMPLE 94 as shown in Scheme 24, following procedures similar to what was described for the synthesis of EXAMPLE 88. The desired compound was obtained as a white solid. ¹H NMR (CD₃OD, 500 MHz) δ 8.56 (1H, d), 8.17 (1H, s), 8.06 (1H, dd), 7.88 (1H, d), 7.56 (1H, t), 7.40 (1H, d), 7.14 (1H, t), 7.09 (1H, dd), 3.23 (2H, t), 2.96 (2H, t); LCMS m/z 382 (M⁺+1).

### Example 95

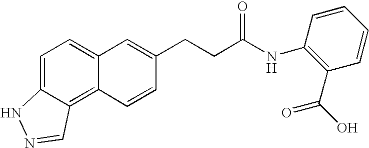

To a solution of aminobromonaphthlene (1.81 g, 8.2 mmol) in 80 mL of dichloromethane at 0° C. were added acetic anhydride (1.15 mL, 12.2 mmol), and triethylamine (2.86 mL, 20 mmol) and a small amount of DMAP. The solution was warmed to RT and stirred for 3 h. The solvent was removed and the residue was dissolved in ethyl acetate, washed with water, 1N HCl, water, 1N NaOH, saturated sodium bicarbonate solution and brine successively. The organic layer was then dried with sodium sulfate and concentrated in vacuo to give the acetamide as a pink solid. This bromide intermediate was subjected to the same Heck reaction and hydrogenation procedures as described earlier, and shown in Scheme 25, to provide the product as a sticky oil. To a solution of this intermediate (86 mg, 0.22 mmol) in 15 mL of chloroform at 0° C., was added dropwise a solution of bromine (14 uL, 42 mg, 0.26 mmol) in 1.5 mL of chloroform. The mixture was stirred at 0° C. for 5 min and quenched with 1% sodium sulfite. Two batches of this intermediate were combined and the aqueous phase was extracted with chloroform three times. The organic phase was washed with saturated sodium bicarbonate solution, and dried over sodium sulfate. A mixture of this bromide intermediate (0.56 g, 1.19 mmol), methyl boronic acid (93 mg, 1.55 mmol), potassium carbonate (494 mg, 3.58 mmol), palladium tetrakistriphenylphosphine (138 mg, 0.12 mmol), 2 mL of water and 20 mL of dioxane, was degassed with argon and heated at 100° C. overnight. After concentration, the residue was purified by Biotage to give a white solid. To a solution of this methylated intermediate (89 mg, 0.22 mmol) in 5 mL of chloroform, were added potassium acetate (44 mg, 0.44 mmol), acetic acid (26 mg, 0.44 mmol), acetic anhydride (45 mg, 0.44 mmol), 18-crown-6 (10 mg), and amylnitrite (74 uL, 0.63 mmol). The mixture was heated at 70° C. overnight. The reaction mixture was then purified by Biotage to give a white solid. To a suspension of this tricyclic acetamide intermediate (58 mg, 0.14 mmol) in 40 mL of methanol, were added sodium ethoxide (226 uL, 21% in methanol). After 5 min, to the mixture was added 10 mL of aqueous 1N lithium hydroxide solution, and the mixture was stirred for 30 min. The solvent was evaporated and the aqueous residue was acidified and extracted with 30% isopropanol in chloroform. After removing the solvent, the residue was purified by RP-HPLC to give the desired product as a white solid. ¹H NMR (DMSO-d₆, 500 MHz) δ 11.2 (1H, s), 8.55 (1H, s), 8.47 (1H, d), 8.24 (1H, d), 7.95 (1H, d), 7.84 (1H, s), 7.67 (1H, d), 7.59 (3H, m), 7.12 (1H, t), 3.12 (2H, t), 2.83 (2H, t); LCMS m/z 360 (M⁺+1).

Moreover, the nicotinic acid receptor has been identified and characterized in WO02/084298A2 published on Oct. 24, 2002 and in Soga, T. et al., Tunaru, S. et al. and Wise, A. et al. (citations above).

Numerous DP receptor antagonist compounds have been published and are useful and included in the methods of the present invention. For example, DP receptor antagonists can be obtained in accordance with WO01/79169 published on Oct. 25, 2001, EP 1305286 published on May 2, 2003, WO02/094830 published on Nov. 28, 2002 and WO03/062200 published on Jul. 31, 2003. Compound AB can be synthesized in accordance with the description set forth in WO01/66520A1 published on Sep. 13, 2001; Compound AC can be synthesized in accordance with the description set forth in WO03/022814A1 published on Mar. 20, 2003, and Compounds AD and AE can be synthesized in accordance with the description set forth in WO03/078409 published on Sep. 25, 2003. Other representative DP antagonist compounds used in the present invention can be synthesized in accordance with the examples provided below.

### DP Example 1

### [5-[(4-Chlorophenyl)thio]-4-(methylsulfonyl)-6,7,8,9-tetrahydropyrido[3,2-b]indolizin-6-yl]acetic acid (Compound G)

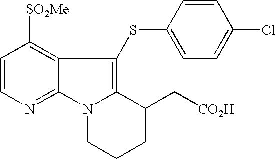

### Step 1 4-Chloronicotinaldehyde

The title compound was prepared as described by F. Marsais et al., J. Heterocyclic Chem., 25, 81 (1988).

### Step 2 4-(Methylthio)nicotinaldehyde

To a solution of NaSMe (9.5 g, 135 mmol) in MeOH (250 mL) was added the 4-chloronicotinaldehyde (13.5 g, 94.4 mmol) of Step 1 in MeOH (250 mL). The reaction mixture was maintained at 60° C. for 15 min. The reaction mixture was poured over NH₄Cl and EtOAc. The organic phase was separated, washed with H₂O and dried over Na₂SO₄. The compound was then purified over silica gel with 50% EtOAc in Hexanes to provide the title compound.

### Step 3 Methyl (2Z)-2-azido-3-[4-(methylthio)pyridin-3-yl]prop-2-enoate

A solution of 4-(methylthio)nicotinealdehyde (4.8 g, 31 mmol) and methyl azidoacetate (9.0 g, 78 mmol) in MeOH (50 mL) was added to a solution of 25% NaOMe in MeOH (16.9 mL, 78 mmol) at −12° C. The internal temperature was monitored and maintained at −10° C. to −12° C. during the 30 min. addition. The resulting mixture was then stirred in an ice bath for several hours, followed by overnight in an ice bath in the cold room. The suspension was then poured onto a mixture of ice and NH₄Cl, and the slurry was filtered after 10 min. of stirring. The product was washed with cold H₂O and was then dried under vacuum to give the title compound as a beige solid, which contained some salts. The compound is then purified over silica gel with EtOAc.

### Step 4 Methyl 4-(methylthio)-1H-pyrrolo[2,3-b]pyridine-2-carboxylate

A suspension of the compound of Step 3 (0.40 g, 1.6 mmol) in xylenes (16 mL) was heated slowly to 140° C. After a period of 15 min. at 140° C., the yellow solution was cooled to room temperature. Precaution must be taken due to the possibility of an exotherme due to the formation of nitrogen. The suspension was then cooled to 0° C., filtered and washed with xylene to provide the title compound.

### Step 5 Ethyl 4-(methylthio)-6-oxo-6,7,8,9-tetrahydropyrido[3,2-b]indolizine-7-carboxylate

To a solution of the compound of Step 4 (0.35 g, 1.6 mmol) in DMF (20 mL) at 0° C. was added NaH (1.2 eq.). After a period of 5 min., nBu₄NI (0.10 g) and ethyl 4-bromobutyrate (0.40 mL). were added. After a period of 1 h at room temperature, the reaction mixture was poured over saturated NH₄Cl and EtOAc. The organic phase was separated, washed with H₂O and dried over NaSO₄. After evaporation the crude product was purified by flash chromatography. The bis ester was then dissolved in THF (7.0 mL) and a 1.06 M of THF solution of potassium tert-butoxide (2.2 mL) was added at 0° C. After a period of 1 h at room temperature, the reaction mixture was then poured over saturated NH₄Cl and EtOAc. The organic phase was separated, dried over Na₂SO₄ and evaporated under reduced pressure to provide the title compound as a mixture of ethyl and methyl ester.

### Step 6 4-(Methylthio)-8,9-dihydropyrido[3,2-b]indolizin-6(7H)-one

To the compound of Step 5, (0.32 g) were added EtOH (8.0 mL) and concentrated HCl (2.0 mL). The resulting suspension was refluxed for 5 h. The reaction mixture was partitioned between EtOAc and Na₂CO₃. The organic phase was separated and evaporated to provide the title compound.

Step 7 Ethyl (2E, 2Z)-[4-(methylthio)-8,9-dihydropyrido[3,2-b]indolizin-6(7H)-ylidene]ethanoate

To a DMF solution (12 mL) of triethyl phosphonoacetate (0.45 g, 2.17 mmol) were added 80% NaH (0.06 g, 2.00 mmol) and the compound of Step 6 (0.22 g, 1.00 mmole). After a period of 4 h at 55° C., the reaction mixture was poured over saturated NH₄Cl and EtOAc. The organic phase was separated and evaporated under reduced pressure. The crude product was purified by flash chromatography to afford the title compound.

### Step 8 Ethyl [4-(methylthio)-6,7,8,9-tetrahydropyrido[3,2-b]indolizin-6-yl]acetate

The compound of Step 7 was dissolved in MeOH—THF using heat for dissolution. To the previous cooled solution was added at room temperature PtO₂ and the resulting mixture was maintained for 18 h under an atmospheric pressure of hydrogen. The reaction mixture was filtered carefully over Celite using CH₂Cl₂. The filtrate was evaporated under reduced pressure to provide the title compound. Alternatively, the compound of Step 7 can be hydrogenated with Pd (OH)₂ in EtOAc at 40 PSI of H₂ for 18 h.

### Step 9 Ethyl [4-(methylsulfonyl)-6,7,8,9-tetrahydropyrido[3,2-b]indolizin-6-yl]acetate

To the compound of Step 8 (0.08 g, 0.27 mmol) in MeOH (3.0 mL) were added Na₂WO₄ (0.10 g) and 30% H₂O₂ (600 μL). After a period of 1 h, the reaction mixture was partitioned between H₂O and EtOAc. The organic phase was washed with H₂O, separated and evaporated. The title compound was purified by flash chromatography.

### Step 10 Ethyl [5-[(4-chlorophenyl)thio]-4-(methylsulfonyl)-6,7,8,9-tetrahydropyrido[3,2-b]indolizin-6-yl]acetate

To a 1,2-dichloroethane solution (2.0 mL) of 4,4′-dichlorodiphenyl disulfide (0.24 g) was added SO₂Cl₂ (50 μL). To the compound of Step 9 (0.05 g) in DMF (2.0 mL) was added the previous mixture (≈180 μL). The reaction was followed by ¹H NMR and maintained at room temperature until no starting material remained. The reaction mixture was poured over saturated NaHCO₃ and EtOAc. The organic phase was separated, evaporated and the title compound purified by flash chromatography.

### Step 11 [5-[(4-Chlorophenyl)thio]-4-(methylsulfonyl)-6,7,8,9-tetrahydropyrido[3,2-b]indolizin-6-yl]acetic acid

To the compound of Step 10 dissolved in a 1/1 mixture of THF-MeOH was added 1N NaOH. After a period of 18 h at room temperature, the reaction mixture was partitioned between saturated NH₄Cl and EtOAc. The organic phase was separated, dried over Na₂SO₄ and evaporated to provide the title compound.

¹H NMR (500 MHz, acetone-d₆) δ 11.00 (bs, 1H), 8.60 (d, 1H), 7.80 (d, 1H), 7.20 (d, 2H), 7.00 (d, 2H), 4.65 (m, 1H), 4.20 (m, 1H), 3.75 (m, 1H), 3.35 (s, 3H), 2.80 to 2.10 (m, 6H).

### DP Example 2

### [5-[(4-Chlorophenyl)thio]-4-(methylthio)-6,7,8,9-tetrahydropyrido[3,2-b]indolizin-6-yl]acetic acid (Compound H)

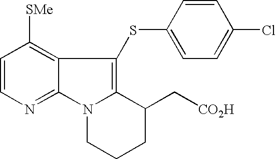

The title compound can be prepared from the compound of Example 1, Step 8 in a similar manner as described in Example 1, Step 10 and 11. m/z 418.

### DP Example 3 [5-[(3,4-Dichlorophenyl)thio]-4-(methylsulfonyl)-6,7,8,9-tetrahydropyrido[3,2-b]indolizin-6-yl]acetic acid (Compound I)

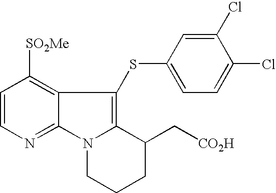

The title compound was prepared as described in Example 1 using bis(3,4-dichlorophenyl)disulfide in Step 10.

¹H NMR (500 MHz, acetone-d₆) δ 8.55 (d, 1H), 7.85 (d, 1H), 7.35 (d, 1H), 7.15 (s, 1H), 6.95 (d, 1H), 4.60 (m, 1H), 4.15 (m, 1H), 3.80 (m, 1H), 3.40 (s, 3H), 2.80 to 2.10 (m, 6H). m/z 484.

The enantiomers were separated on a Chiralecel OD column 25 cm×20 mm using 30% isopropanol 17% ethanol 0.2% acetic acid in hexane, flow rate 8 ml/min. Their pureties were verified on a Chiralecel OD column 25 cm×4.6 mm using 35% isopropanol 0.2% acetic acid in hexane, flow rate 1.0 ml/min. More mobile enantiomer Tr=9.7 min, less mobile enantiomer Tr 11.1 min.

### DP Example 4

### [5-(4-Chlorobenzoyl)-4-(methylsulfonyl)-6,7,8,9-tetrahydropyrido[3,2-b]indolizin-6-yl]acetic acid (Compound J)

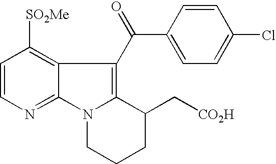

### Step 1 Ethyl [5-(4-chlorobenzoyl)-4-(methylthio)-6,7,8,9-tetrahydropyrido[3,2-b]indolizin-6-yl]acetate

To a solution of 4-chlorobenzoyl chloride (0.30 g, 1.7 mmol) in 1,2-dichloethane (6.0 mL) was added AlCl₃ (0.24 g, 1.8 mmole). After a period of 5 min. a solution of ethyl [4-(methylthio)-6,7,8,9-tetrahydropyrido[3,2-b]indolizin-6-yl]acetate from Example 1 Step 8 (0.15 g, 0.47 mmole) in 1,2-dichloroethane (6.0 mL) was added to the previous mixture. After a period of 4 h, at 80° C., the reaction mixture was partitioned between EtOAc and NaHCO₃. The organic phase was separated, dried over Na₂SO₄ and evaporated. The title compound was purified by flash chromatography.

### Step 2 Ethyl [5-(4-chlorobenzoyl)-4-(methylsulfonyl)-6,7,8,9-tetrahydropyrido[3,2-b]indolizin-6-yl]acetate

To a solution of ethyl[5-(4-chlorobenzoyl)-4-(methylthio)-6,7,8,9-tetrahydropyrido[3,2-b]indolizin-6-yl]acetate (0.12 g, 0.27 mmole) in MeOH (5.0 mL) were added Na₂WO₄ (0.1 g) and 30% H₂O₂ (300 μL). The reaction mixture was stirred at 55° C. for 1 h. The reaction mixture was then partitioned between H₂O and EtOAc. The organic phase was washed with H₂O, dried over Na₂SO₄ and evaporated. The title compound was purified by flash chromatography.

### Step 3 [5-(4-Chlorobenzoyl)-4-(methylsulfonyl)-6,7,8,9-tetrahydropyrido[3,2-b]indolizin-6-yl]acetic acid

Ethyl [5-(4-chlorobenzoyl)-4-(methylsulfonyl)-6,7-8,9-tetrahydropyrido[3,2-b]indolizin-6-yl]acetate was treated as described in Example 1 Step 11 to provide the title compound.

¹H NMR (500 MHz, acetone-d₆) δ 8.55 (d, 1H), 7.90 (d, 2H), 7.65 (d, 1H), 7.45 (d, 2H), 4.55 (m, 1H), 4.25 (m, 1H), 3.45 (m, 1H), 3.20 (s, 3H), 2.05 to 3.00 (m, 6H). m/z 446.

### DP Example 5

### [5-(4-Bromophenyl)thio]-4-(methylsulfonyl)-6,7,8,9-tetrahydropyrido[3,2-b]indolizin-6-yl]acetic acid (Compound K)

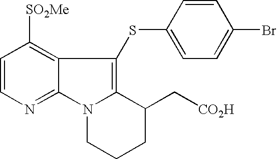

The title compound was prepared as described in Example 1 using 4,4′-dibromodiphenyl disulfide.

¹H NMR (500 MHz, Acetone-d6) δ 8.60 (d, 1H), 7.80 (d, 1H), 7.35 (d, 2H), 7.00 (d, 2H), 4.65 (m, 1H), 4.20 (m, 1H), 3.80 (m, 1H), 3.35 (s, 3H), 2.80 to 2.10 (m, 6H).

### DP Example 6 Method-1

### [9-[(3,4-Dichlorophenyl)thio]-1-(methylsulfonyl)-7,8-dihydro-6H-pyrido[3,4-b]pyrrolizin-8-yl]acetic acid (Compound L)

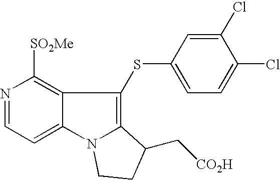

### Step 1 2-(Methylthio)nicotinaldehyde

The title compound was prepared from 2-bromonicotinaldehyde (A. Numata Synthesis 1999 p. 306) as described in Example 1 Step 2 except the solution was heated at 55° C. for 2 hr.

### Step 2 Methyl (2Z)-2-azido-3-[2-(methylthio)pyridin-3-yl]prop-2-enoate

The title compound was prepared as described in Example 1 Step 3.

### Step 3 Methyl 4-(methylthio)-1H-pyrrolo[3,2-c]pyridine-2-carboxylate

A solution of methyl (2Z)-2-azido-3-[2-(methylthio)pyridin-3-yl]prop-2-enoate (1.00 g, 4.00 mmol) in mesitylene (50 mL) was heated at 160° C. for a period of 1 h. The reaction mixture was cooled to room temperature then to 0° C., the precipitate was filtered and washed with cold mesitylene to provide the title compound.

### Step 4 Methyl 1-(methylthio)-8-oxo-7,8-dihydro-6H-pyrido[3,4-b]pyrrolizine-7-carboxylate

To a suspension of methyl 4-(methylthio)-1H-pyrrolo[3,2-c]pyridine-2-carboxylate (0.30 g, 1.35 mmol) in THF (3 mL)-toluene (12.0 mL) were added a 1.06 M THF solution of potassium tert-butoxide (1.42 mL/1.41 mmol) and methyl acrylate (300 μL). The resulting mixture was heated at 80° C. for 18 h. The mixture was partitioned between EtOAc and NH₄Cl, and filtered through Celite. The organic phase was separated, dried over Na₂SO₄ and filtered, to provide the title compound.

### Step 5 1-(Methylthio)-6,7-dihydro-8H-pyrido[3,4-b]pyrrolizin-8-one

Methyl 1-(methylthio)-8-oxo-7,8-dihydro-6H-pyrido[3,4-b]pyrrolizine-7-carboxylate was converted to the title compound as described in Example 1 Step 6.

### Step 6 Methyl [8-hydroxy-1-(methylthio)-7,8-dihydro-6H-pyrido[3,4-b]pyrrolizin-8-yl]acetate

A mixture of 1-(methylthio)-6,7-dihydro-8H-pyrido[3,4-b]pyrrolizin-8-one (0.15 g, 0.68 mmol), methyl bromoacetate (0.34 mL), Zn-Cu (0.226 g) in THF (3.0 mL) was sonicated for 2 h. The mixture was then heated at 60° C. for 5 min. until completion of the reaction. The reaction mixture was partitioned between EtOAc and NH₄Cl. The organic phase was separated, dried over Na₂SO₄, filtered and evaporated under reduced pressure to provide the title compound. The compound was purified by flash chromatography.

### Step 7 Methyl [1-(methylthio)-7,8-dihydro-6H-pyrido[3,4-b]pyrrolizin-8-yl]acetate

To NaI (0.300 g) in CH₃CN (3.2 mL) was added TMSCl (0.266 mL). This mixture was added to a suspension of methyl [8-hydroxy-1-(methylthio)-7,8-dihydro-6H-pyrido[3,4-b]pyrrolizin-8-yl]acetate (0.15 g, 0.515 mmol) in CH₃CN (1.5 mL), in a water bath. After a period of 0.5 h, the reaction mixture was partitioned between EtOAc and NaHCO₃. The organic phase was separated, washed with sodium thiosulphate, dried over MgSO₄ and evaporated. The title compound was purified by flash chromatography.

### Step 8 Methyl [1-(methylsulfonyl)-7,8-dihydro-6H-pyrido[3,4-b]pyrrolizin-8-yl]acetate

Methyl [1-(methylthio)-7,8-dihydro-6H-pyrido[3,4-b]pyrrolizin-8-yl]acetate was converted to the title compound as described in Example 1 Step 9.

### Step 9 [9-[(3,4-Dichlorophenyl)thio]-1-(methylsulfonyl)-7,8-dihydro-6H-pyrido[3,4-b]pyrrolizin-8-yL]acetic acid

Methyl [1-(methylsulfonyl)-7,8-dihydro-6H-pyrido[3,4-b]pyrrolizin-8-yl]acetate was converted to the title compound as described in Example 1, Steps 10 and 11, using bis(3,4-dichlorophenyl)disulfide in Step 10.

¹H NMR (500 MHz, acetone-d₆) δ 8.35 (d, 1H) 7.80 (d, 1H), 7. 35 (d, 1H), 7.15 (s, 1H), 6.95 (d, 1H), 4.55 (m, 1H), 4.35 (m, 1H), 3.90 (m, 1H), 3.30 (s, 3H), 3.15 (m, 1H), 3.05 (m, 1H), 2.80 (m, 1H), 2.50 (m, 1H).

### DP Example 6 Method-2

### [9-[(3,4-Dichlorophenyl)thio]-1-(methylsulfonyl)-7,8-dihydro-6H-pyrido[3,4-b]pyrrolizin-8-yl]acetic acid

### Step 1 1-(Methylthio)-7,8-dihydro-6H-pyrido[3,4-b]pyrrolizin-8-ol

To a suspension of 1-(methylthio)-6,7-dihydro-8H-pyrido[3,4-b]pyrrolizin-8-one from Example 6, Method-1 Step 5 (0.55 g, 2.2 mmol) in EtOH (10 mL)-THF (1 mL) was added NaBH₄ (0.10 g, 2.6 mmol) at 0° C. After a period of 30 min. at room temperature, the reaction was quenched by the addition of acetone. The solvents were evaporated under reduced pressure and EtOAC and H₂O were added to the residue. The organic phase was separated, dried over MgSO₄ and evaporated. The title compound was washed with EtOAc/Hexane and filtered.

### Step 2 Dimethyl 2-[1-(methylthio)-7,8-dihydro-6H-pyrido[3,4-b]pyrrolizin-8-yl]malonate

To a suspension of 1-(methylthio)-7,8-dihydro-6H-pyrido[3,4-b]pyrrolizin-8-ol (0.54 g, 2.1 mmol) in THF (10 mL) at −78° C. were added 1M NaHMDS in THF (2.35 mL, 2.4 mmol) and diphenyl chlorophosphate (0.53 mL, 2.6 mmol). After a period of 30 min. dimethyl malonate (0.73 mL, 6.4 mmol) and 1M NaHMDS in THF (6.8 mL, 6.8 mmol) were added. The reaction mixture was brought to 0° C. and then to room temperature. The mixture was then partitioned between ETOAc and NH₄Cl. The organic phase was dried over MgSO₄, filtered and evaporated. The title compound was purified by flash chromatography.

### Step 3 Methyl [1-(methylthio)-7,8-dihydro-6H-pyrido[3,4-b]pyrrolizin-8-yl]-acetate

To a mixture of dimethyl 2-[1-(methylthio)-7,8-dihydro-6H-pyrido[3,4-b]pyrrolizin-8-yl]malonate (0.59 g, 2.17 mmol) and DMSO (4 mL) was added NaCl (0.45 g) in H2O (0.45 mL). After a period of 18 h at 150° C., the reaction mixture was partitioned between ETOAc and H2O. The organic phase was separated, dried over Na2SO4 and evaporated. The title compound was then purified by flash chromatography.

### Step 4 [9-[(3,4-Dichlorophenyl)thio]-1-(methylsulfonyl)-7,8-dihydro-6H-pyrido[3,4-b]pyrrolizin-8-yl]acetic acid

The title compound was obtained from methyl [1-(methylthio)-7,8-dihydro-6H-pyrido[3,4-b]pyrrolizin-8-yl]acetate as described in Example 6, Method-1, Steps 8 to 9.

### DP Example 7

### [10-[(3,4-Dichlorophenyl)sulfanyl]-1-(methylsulfonyl)-6,7,8,9-tetrahydropyrido[3,4-b]indolizin-9-yl]acetic acid (Compound M)

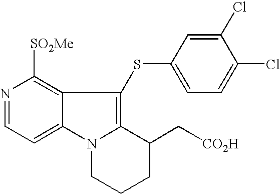

### Step 1 Ethyl [1-(methylsulfonyl)-6,7,8,9-tetrahydropyrido[3,4-b]indolizin-9-yl]acetate

The title compound was prepared from the product of Example 6, Step 3 in the same manner as described in Example 1, Steps 5 to 9.

### Step 2 [10-[(3,4-Dichlorophenyl)sulfanyl]-1-(methylsulfonyl)-6,7,8,9-tetrahydropyrido[3,4-b]indolizin-9-yl]acetic acid

The product of Step 1 was converted to the title compound in the same manner as Example 1, Steps 10-11, using bis(3,4-dichlorophenyl)disulfide in Step 10.

MS M+1=485.

### DP Example 8

### (4-(Methylsulfonyl)-5-{[4-(trifluoromethyl)phenyl]thio}-6,7,8,9-tetrahydropyrido[3,2-b]indolizin-6-yl)acetic acid (Compound N)

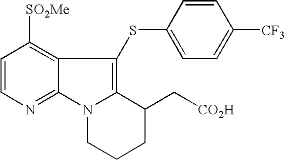

The title compound was prepared as described in Example 1 using bis[4-trifluoromethyl)phenyl]disulfide.

¹H NMR (500 MHz, acetone-d₆) δ 8.55 (d, 1H), 7.75 (d, 1H), 7.45 (d, 2H), 7.15 (d, 2H), 4.55 (m, 1H), 4.15 (m, 1H), 3.80 (m, 1H), 3.30 (s, 3H), 2.80 to 2.10 (m, 6H).

m/z 513 (M+1).

### DP Example 9

### [5-Chloro-4-fluorophenyl)thio]-4-(methylsulfonyl)-6,7,8,9-tetrahydropyrido[3,2b]indolizin-6-yl]acetic acid (Compound O)

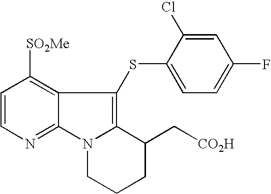

The title compound was prepared as described in Example 1 using bis(2-chloro-4-fluorophenyl)disulfide. m/z 469 (M+1).

### DP Example 10

### [4-(Methylsulfonyl)-5-(2-naphthylthio)-6,7,8,9-tetrahydropyrido[3,2-b]indolizin-6-yl]acetic acid (Compound P)

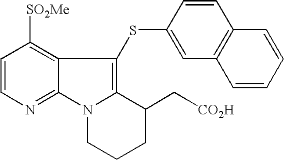

The title compound was prepared as described in Example 1 using di(2-naphthyl) disulfide.

M/z 467 (M+1).

### DP Example 11

### [5-[(2,3-Dichlorophenyl)thio]4-(methylsulfonyl)-6,7,8,9-tetrahydropyrido[3,2-b]indolizin-6-yl]acetic acid (Compound Q)

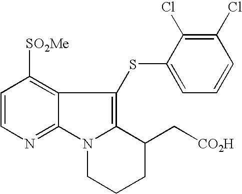

The title compound was prepared as described in Example 1 using bis(2,3-dichlorophenyl)disulfide.

¹H NMR (500 MHz, acetone-d₆) δ 8.85 (d, 1H), 7.80 (d, 1H), 7.30 (d, 1H), 7.00 (t, 1H), 6.60 (d, 1H), 4.60 (m, 1H), 4.20 (m, 1H), 3.80 (m, 1H), 3.40 (s, 3H), 2.80 to 2.10 (m, 6H).

### DP Example 12

### [5-[(4-Methylphenyl)thio]-4-(methylsulfonyl)-6,7,8,9-tetrahydropyrido[3,2-b]indolizin-6-yl]acetic acid (Compound R)

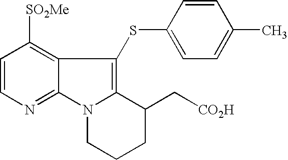

The title compound was prepared as described in Example 1 using p-tolyl disulfide.

¹H NMR (500 MHz, acetone-d₆) δ 8.55 (d, 1H), 7.80 (d, 1H), 6.95 (m, 4H), 4.60 (m, 1H), 4.15 (m, 1H), 3.80 (m, 1H), 3.35 (s, 3H), 2.80 to 2.10 (m, 6H).

### DP Example 13

### [4-(Methylsulfonyl)-5-(phenylthio)-6,7,8,9-tetrahydropyrido[3,2-b]indolizin-6-yl]acetic acid (Compound S)

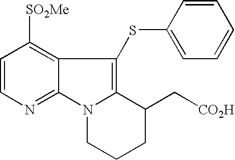

The title compound was prepared as described in Example 1 using diphenyl disulfide.

¹H NMR (500 MHz, acetone-d₆) δ 8.55 (d, 1H), 7.80 (d, 1H), 7.15 to 6.90 (m, 5H), 4.60 (m, 1H), 4.15 (m, 1H), 3.75 (m, 1H), 3.30 (s, 3H), 2.80 to 2.10 (m, 6H).

### DP Example 14

### [5-[(2,4-Dichlorophenyl)thio]-4-(methylsulfonyl)-6,7,8,9-tetrahydropyrido[3,2-b]indolizin-6-yl]acetic acid (Compound T)

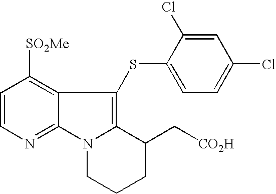

The title compound was prepared as described in Example 1 using bis(2,4-dichlorophenyl)disulfide. The disulfide was prepared from 2,4-dichlorothiophenyl using Br₂ in ether.

¹H NMR (500 MHz, acetone-d₆) δ 8.55 (d, 1H), 7.85 (d, 1H), 7.35 (s, 1H), 7.00 (d, 1H), 6.65 (d, 1H), 4.55 (m, 1H), 4.15 (m, 1H), 3.80 (m, 1H), 3.35 (s, 3H), 2.80 to 2.10 (m, 6H).

### DP Example 15

### [5-[(4-Chlorophenyl)thio]-4-(methylsulfonyl)-6,7,8,9-tetrahydropyrido[4,3-b]indolizin-6-yl]acetic acid (Compound U)

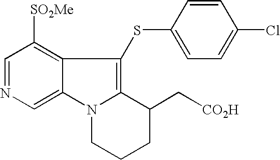

The title compound was prepared as described in Example 1 from 3-chloronicotinaldehyde (Heterocycles p. 151, 1993) except the terminal cyclization was performed by adding the azide to decalin at reflux.

¹H NMR (500 MHz, acetone-d₆) δ 9.20 (s, 1H), 8.85 (s, 1H), 7.20 (d, 2H), 7.00 (d, 2H), 4.70 (m, 1H), 4.30 (m, 1H), 3.75 (m, 1H), 3.35 (s, 3H), 2.80 to 2.10 (m, 6H).

### DP Example 16

### [9-[(4-Chlorophenyl)thio]-1-(methylsulfonyl)-7,8-dihydro-6H-pyrido[3,4-b]pyrrolizin-8-yl]acetic acid (Compound V)

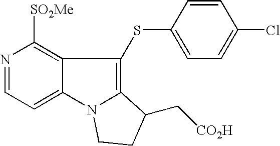

The title compound was prepared from the product of Example 6 Method 1 Step 8, as described in the procedures outlined in Example 1 Steps 10 and 11, using bis(4-chlorophenyl)disulfide in Step 10.

¹H NMR (500 MHz, acetone-d₆) δ 8.25-8.3 (m, 1H), 7.71-7.75 (m, 1H), 7.12-7.17 (m, 2H), 6.97-7.04 (m, 2H), 4.45-4.51 (m, 1H), 4.32-4.39 (m, 1H), 3.73-3.80 (m, 1H), 3.29 (s, 3H), 3.15-3.21 (m, 1H), 2.99-3.08 (m, 1H), 2.66-2.73 (m, 1H), 2.46-2.54 (m, 1H).

### DP Example 17

### (−)-[(4-Chlorobenzyl)-7-fluoro-5-methanesulfonyl)-1,2,3,4-tetrahydrocyclopenta[b]indol-3-yl]acetic acid (Compound E)

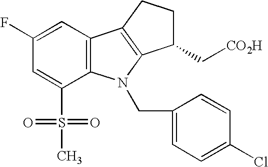

### Step 1: (+/−)-(7-Fluoro-1,2,3,4-tetrahydrocyclopenta[b]indol-3-yl)acetic acid ethyl ester

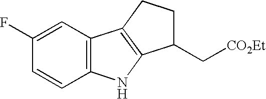

A solution of 10.00 g of 4-fluoro-2-iodoaniline, 6.57 g of ethyl 2-(2-oxocyclopentyl)acetate and 121 mg of p-toluenesulfonic acid in 100 ml of benzene was refluxed with a Dean-Stark trap under a N₂ atmosphere for 24 h. After this time, the benzene was removed under distillation. Then, 60 ml of DMF was added and the solution was degassed before 19 ml of Hunig's base followed by 405 mg of Pd(OAc)₂ were added successively. The solution was heated to 115° C. for 3 h, then cooled to room temperature. To quench the reaction, 300 ml of 1 N HCl and 200 ml of ethyl acetate were added and the mixture was filtered through Celite. The phases were separated and the acidic phase was extracted twice with 200 ml of ethyl acetate. The organic layers were combined, washed with brine, dried over anhydrous Na₂SO₄, filtered through Celite and concentrated. The crude material was further purified by flash chromatography eluting with 100% toluene, to provide the title compound.

¹H NMR (acetone-d₆) δ 9.76 (br s, 1H), 7.34 (dd, 1H), 7.03 (d, 1H), 6.78 (td, 1H), 4.14 (q, 2H), 3.57 (m, 1H), 2.85-2.55 (m, 5H), 2.15 (m, 1H), 1.22 (t, 3H).

### Step 2: (+/−)-(7-Fluoro-1, 2,3,4-tetrahydrocyclopenta[b]indol-3-yl)acetic acid

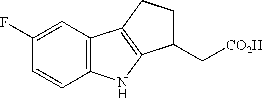

To a solution of 1.24 g of the ester from Step 1 in 14 mL of tetrahydrofuran (THF) at room temperature, 7 mL of MeOH followed by 7 mL of 2N NaOH were added. After 2.5 h, the reaction mixture was poured into a separatory funnel containing ethyl acetate (EtOAc)/1N HCl. The phases were separated and the acidic phase was extracted twice with EtOAc. The organic layers were combined, washed with brine, dried over anhydrous Na₂SO₄ and evaporated to dryness to yield a crude oil that was used as such in the next step (>90% purity).

¹H NMR (acetone-d₆) δ 10.90 (br s, 1H), 9.77 (br s, 1H), 7.34 (dd, 1H), 7.04 (dd, 1H), 6.79 (td, 1H), 3.56 (m, 1H), 2.90-2.50 (m, 5H), 2.16 (m, 1H). MS (-APCI) m/z 232.2 (M−H)⁻.

### Step 3: (+/−)-(5-bromo-7-fluoro-1,2,3,4-tetrahydrocyclopenta[b]indol-3-yl)acetic acid

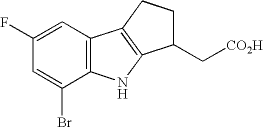

To a solution of 2.20 g of the acid from Step 2 (>90% purity) in 30 mL of pyridine, 6.85 g of pyridinium tribromide (90% purity) was added at −40° C. The suspension was stirred for 10 min at 0° C. and warmed to room temperature for 30 min. Then, the solvent was removed without heating under high vacuum. The crude material was dissolved in 40 mL of AcOH and 2.88 g of Zn dust was added portion wise to the cold solution at 0° C. The suspension was stirred for 15 min at 15° C. and warmed to room temperature for an additional 15 min. At this time, the reaction mixture was quenched by the addition of 1N HCl and this mixture was poured into a separatory funnel containing brine/EtOAc. The layers were separated and the organic layer was washed with water, brine, dried over anhydrous Na₂SO₄ and concentrated. This material was used without further purification in the next step.

¹H NMR (acetone-d₆) δ 10.77 (br s, 1H), 9.84 (br s, 1H), 7.09 (m, 2H), 3.60 (m, 1H), 2.95-2.65 (m, 4H), 2.56 (dd, 1H), 2.19 (m, 1H).

### Step 4: (+/−)-[5-bromo-4-(4-chlorobenzyl)-7-fluoro-1,2,3,4-tetrahydrocyclopenta[b]indol-3-yl]-acetic acid

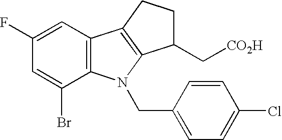

To a solution of 2.13 g of the acid from Step 3 in 10 mL of THF, a solution of diazomethane in ether was added in excess until complete consumption of the acid as monitored on TLC. Then, the solvents were removed under vacuum. To a solution of the crude methyl ester thus formed in 20 mL of DMF, 539 mg of a NaH suspension (60% in oil) was added at −78° C. The suspension was stirred for 10 min at 0° C., cooled again to −78° C. and treated with 1.70 g of 4-chlorobenzyl bromide. After 5 min, the temperature was warmed to 0° C. and the mixture was stirred for 20 min. At this time, the reaction was quenched by the addition of 2 mL of AcOH and this mixture was poured into a separatory funnel containing 1N HCl/EtOAc. The layers were separated and the organic layer was washed with brine, dried over anhydrous Na₂SO₄ and concentrated. The alkylated material was hydrolyzed using the procedure described in Step 2. The crude material was further purified by trituration with EtOAc/hexanes to provide the title compound.

¹H NMR (acetone-d₆) δ 10.70 (br s, 1H), 7.31 (d, 2H), 7.18 (d, 1H), 7.06 (d, 1H), 6.92 (d, 2H), 5.90 (d, 1H), 5.74 (d, 1H), 3.61 (m, 1H), 3.00-2.70 (m, 3H), 2.65 (dd, 1H), 2.39 (dd, 1H), 2.26 (m, 1H). MS (-APCI) m/z 436.3, 434.5 (M−H)⁻.

### Step 5: (+)-[5-bromo-4-(4-chlorobenzyl)-7-fluoro-1,2,3,4-tetrahydrocyclopenta[b]indol-3-yl}acetic acid

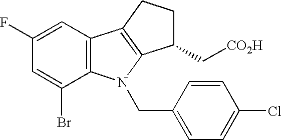

To a solution of 2.35 g of the acid of Step 4 in 130 mL of EtOH at 80° C., was added 780 μL of (S)-(-)-1-(1-naphthyl)ethylamine. The solution was cooled to room temperature and stirred overnight. The salt recovered (1.7 g) was recrystallized again with 200 mL of EtOH. After filtration, the white solid salt obtained was neutralized with 1N HCl and the product was extracted with EtOAc. The organic layer was washed with brine, dried over anhydrous Na₂SO₄ and concentrated. The material was filtered over a pad of SiO₂ by eluting with EtOAc to produce the title enantiomer. Retention times of the two enantiomers were respectively 7.5 min and 9.4 min [ChiralPak AD column, hexane/2-propanol/acetic acid (95:5:0.1)]. The more polar enantiomer was in 98% ee.

ee=98%; Retention time=9.4 min [ChiralPak AD column: 250×4.6 mm, hexanes/2-propanol/acetic:acid (75:25:0.1)]; [α]_{D}²¹=+39.2° (c 1.0, MeOH).

### Step 6: (−)-[4-(4-chlorobenzyl)-7-fluoro-5-(methanesulfonyl)-1,2,3,4-tetrahydrocyclopenta[b]-indol-3-yl}acetic acid and sodium salt

The acid from Step 5 (15.4 g) was first esterified with diazomethane. The sulfonylation was accomplished by mixing the ester thus formed with 16.3 g of methanesulfinic acid sodium salt and 30.2 g of CuI (I) in N-methylpyrrolidinone. The suspension was degassed under a flow of N₂, heated to 150° C. and stirred for 3 h, then cooled to room temperature. To quench the reaction, 500 ml of ethyl acetate and 500 ml of hexanes were added and the mixture was filtered through a pad of SiO₂ by eluting with EtOAc. The organic phases were concentrated. The crude oil was dissolved with EtOAc, washed three times with water one time with brine, dried over anhydrous Na₂SO₄, filtered and concentrated. The crude material was further purified by flash chromatography eluting with a gradient from 100% toluene to 50% toluene in EtOAc, to provide 14 g of the sulfonated ester, which was hydrolyzed using the procedure described in Step 2. The title compound was obtained after two successive recrystallizations: isopropyl acetate/heptane followed by CH₂Cl₂/hexanes.

¹H NMR (500 MHz acetone-d₆) δ 10.73 (br s, 1H), 7.57 (d, 2H, J=8.8 Hz), 7.31 (m, 1H), 7.29 (m, 1H), 6.84 (d, 2H, J=8.8 Hz), 6.29 (d, 1H, J_{AB}=17.8 Hz), 5.79 (d, 1H, J_{AB}=17.8 Hz), 3.43 (m, 1H), 2.98 (s, 3H), 2.94 (m, 1H), 2.85-2.65 (m, 3H), 2.42 (dd, 1H, J₁=16.1 Hz, J₂=10.3 Hz), 2.27 (m, 1H). ¹³C NMR (125 MHz acetone-d₆) δ 173.0, 156.5 (d, JC_{F}=237 Hz), 153.9, 139.2, 133.7, 133.3, 130.0 (d, J_{CF}=8.9 Hz), 129.6, 128.2, 127.5 (d, J_{CF}=7.6 Hz), 122.2 (d, J_{CF}=4.2 Hz), 112.3 (d, J_{CF}=29.4 Hz), 111.0 (d, J_{CF}=22.6 Hz), 50.8, 44.7, 38.6, 36.6, 36.5, 23.3. MS (-APCI) m/z 436.1, 434.1 (M−H)⁻.

ee=97%; Retention time=15.3 min [ChiralCel OD column: 250×4.6 mm, hexanes/2-propanol/ethanol/acetic acid (90:5:5:0.2)]; [α]_{D}²¹=−29.3° (c 1.0, MeOH). Mp 175.0° C.

The sodium salt was prepared by the treatment of 6.45 g (14.80 mmol) of the above acid compound in EtOH (100 mL) with 14.80 mL of an aqueous 1N NaOH solution. The organic solvent was removed under vacuum and the crude solid was dissolved in 1.2 L of isopropyl alcohol under reflux. The final volume was reduced to 500 mL by distillation of the solvent. The sodium salt crystallized by cooling to rt. The crystalline sodium salt was suspended in H₂O, frozen with a dry ice bath and lyophilized under high vacuum to give the title compound as the sodium salt.

¹H NMR (500 MHz DMSO-d₆) δ 7.63 (dd, 1H, J₁=8.5 Hz, J₂=2.6 Hz), 7.47 (dd, 1H, J₁=9.7 Hz, J₂=2.6 Hz), 7.33 (d, 2H, J=8.4 Hz), 6.70 (d, 2H, J=8.4 Hz), 6.06 (d, 1H, J_{AB}=17.9 Hz), 5.76 (d, 1H, J_{AB}=17.9 Hz), 3.29 (m, 1H), 3.08 (s, 3H), 2.80 (m, 1H), 2.69 (m, 1H), 2.55 (m, 1H), 2.18 (m, 2H), 1.93 (dd, 1H, J₁=14.4 Hz, J₂=9.7 Hz).

### DP Example 17A

### Alternative procedure for (+/−)-[5-bromo-4-(4-chlorobenzyl)-7-fluoro-1,2,3,4-tetrahydrocyclopenta[b]indol-3-yl]acetic acid (Example 17 Step 4)

### Step 1: (+/−)-7-fluoro-1,2,3,4-tetrahydrocyclopenta[b]indol-3-yl)acetic acid dicyclohexylamine (DCHA) salt

A 0.526 M solution of 2-bromo-4-fluoroaniline in xylene along with ethyl (2-oxocyclopentyl)acetate (1.5 eq) and sulfuric acid (0.02 eq) was heated to reflux for 20 hours. Water was azeotropically removed with a Dean-Stark apparatus. The reaction was followed by NMR and after 20 hours, an 80-85% conversion to the desired imine intermediate was generally observed. The reaction mixture was washed with 1M sodium bicarbonate (0.2 volumes) for 15 minutes and the organic fraction was evaporated. The remaining syrup was distilled under vacuum (0.5 mm Hg). Residual xylenes distilled at 30° C., then excess ketone and unreacted aniline were recovered in the 50-110° C. range; the imine was recovered in the 110-180° C. fraction as a light brown clear liquid with 83% purity.

The imine intermediate was then added to a degased mixture of potassium acetate (3 eq), tetra-n-butylammonium chloride monohydrate (1 eq), palladium acetate (0.03 eq) and N,N-dimethylacetamide (final concentration of imine=0.365 M). The reaction mixture was heated to 115° C. for 5 hours and allowed to cool to room temperature. 3N KOH (3 eq) was then added and the mixture was stirred at room temperature for 1 hour. The reaction mixture was diluted with water (1.0 volume), washed with toluene (3×0.75 volume). The aqueous phase was acidified to pH 1 with 3N HCl and extracted with tertbutyl methyl ether (2×0.75 volume). The combined organic fractions were washed with water (0.75 volume). To the clear light brown solution was added dicyclohexylamine (1 eq) and the solution was stirred at room temperature for 16 hours. The salt was filtered, washed with ethyl acetate, tertbutyl methyl ether and allowed to dry to give the title compound. Assay: 94 A %.

1H NMR (500 mHz, CDCl3): δ 9.24 (s, 1H), 7.16-7.08 (m, 2H), 6.82 (t, 1H), 6.2 (br, 2H), 3.6-3.5 (m, 1H), 3.04-2.97 (m, 2H), 2.88-2.70 (m, 3H), 2.66 (dd, 1H), 2.45-2.37 (m, 1H), 2.13-2.05 (m, 2.05), 1.83 (d, 4H), 1.67 (d, 2H), 1.55-1.43 (m, 4H), 1.33-1.11 (m, 6H).

### Step 2: (+/−)-(5-bromo-7-fluoro-1,2,3,4-tetrahydrocyclopenta[b]indol-3-yl)acetic acid

A slurry of the DCHA salt from Step 1 above in dichloromethane (0.241 M solution) was cooled to −20 to −15° C. Pyridine (2 eq.) was added in one shot and to the slurry was added dropwise bromine (2.5 eq.) over 30 to 45 minutes maintaining the temperature between −20° C. and −15° C. (At about ⅓ addition of bromine, the reaction mixture was thick and an efficient stirring was needed. Eventually, at about ½ addition of bromine, the mixture became “loose” again.) After completion of the addition, the reaction mixture was aged for one additional hour at −15° C. Acetic acid (3.04 eq.) was then added over 5 minutes and zinc dust (3.04 eq.) was added portion wise. (A portion of zinc was added at −15° C. and the mixture was aged for about 5 minutes to ensure that the exotherm was going (about −15° C. to −10° C.)). This operation was repeated with about 5 shots of zinc over about 30 min. When no more exotherm was observed, the remaining zinc was added faster. The whole operation took around 30 to 45 minutes.

After completion of the addition, the batch was warmed to room temperature, aged 1 hour and concentrated. The reaction mixture was switched to methyl t-butyl ether (MTBE, 0.8 volume) and a 10% aqueous acetic acid solution (0.8 volume) was added. The mixture (crystallization of salts, e.g pyridium) was aged at room temperature for 1 hour and filtered through solka-floc. The pad of solka-floc was rinsed with MTBE (ca. 0.2 volume) and the filtrate (biphasic, MTBE/aqueous) was transferred into an extractor. The organic phase was washed with water (0.8 volume). The MTBE extract was concentrated and switched to isopropyl alcohol (IPA, 0.25 volume) to crystallize the compound. Water (0.25 volumes) was added and the batch was aged for 1 hour. Additional water (0.33 volumes) was added over 1 hour. After completion of the water addition, the batch was aged for one additional hour, filtered, and rinse with 30/70 IPA/Water (0.15 volumes). Crystallized bromoacid was dried in the oven at +45° C.

### Step 3: (+/−)-[5-bromo-4-(4-chlorobenzyl)-7-fluoro-1,2,3,4-tetrahydrocyclopenta[b]indol-3-yl]-acetic acid

The bromoacid of Step 2 was dissolved in dimethylacetamide (0.416 M solution) and cesium carbonate (2.5 eq.) was added in one portion. To the slurry was added in one portion 4-chlorobenzyl chloride (2.5 eq.) and the batch was heated to 50° C. for 20 h. The batch was cooled to r.t. and sodium hydroxide 5N (4.00 eq.) was added over 5 minutes (temperature rose to +40° C.). The reaction was aged at 50° C. for ca. 3 hours, cooled to room temperature and transferred into an L extractor. The solution was diluted with isopropylacetate (IPAc, 2 volumes) and cooled to +15° C. The solution was acidified with 5N HCl to pH˜2. Layers were separated and the organic layer was washed with water (2×2 volumes). IPAc solution was concentrated and switched to IPA (0.8 volumes) to crystallize the product. Water (8 L) was added over 2 hours and the batch was filtered to give the title compound. The batch can be dried in the oven at +40° C. for 24 hours.

### DP Example 18

### (+/−)-{4-[1-(4-Chlorophenyl)ethyl]-7-fluoro-5-methanesulfonyl-1,2,3,4-tetrahydrocyclopenta[b]indol-3-yl}acetic acid (Compound X)

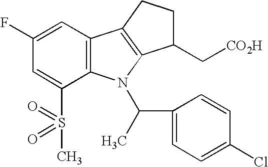

The title compound was synthesized in accordance with the description provided in PCT WO03/062200 published on Jul. 30, 2003.

### DP Example 19

### (+/−)-[9-(4-Chlorobenzyl)-6-fluoro-methanesulfonyl-2,3,4,9-tetrahydro-1H-carbazol-1-yl]acetic acid (Compound Y)

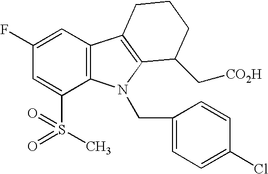

The title compound was synthesized in accordance with the description provided in PCT WO03/062200 published on Jul. 30, 2003.

### DP Example 20

### [4-(4-Chlorobenzyl)-7-fluoro-5-methanesulfonyl-1-oxo-1,2,3,4-tetrahydrocyclopenta[b]indol-3-yl]acetic acid (Compound Z)

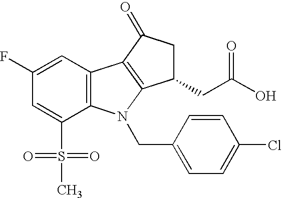

The title compound was synthesized in accordance with the description provided in PCT WO03/062200 published on Jul. 30, 2003:

### DP Example 21

### {9-[(3,4-Dichlorophenyl)thio]-1-isopropyl-7,8-dihydro-6H-pyrido[3,4-b]pyrrolizin-8-yl}acetic acid (Enantiomer A and Enantiomer B) (Compound AA)

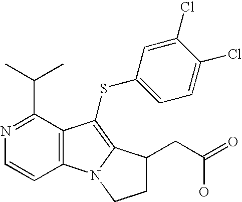

### Step 1 2-Chloronicotinaldehyde

To a solution of diisopropyl amine (110 mL, 780 mmol) in THF (500 mL) was added a 2.5 M hexanes solution of n-BuLi (300 mL, 750 mmol) at −40° C. After 5 min, the reaction mixture was cooled to −95° C. then DMPU (15 mL) and 2-chloropyridine (50 mL, 532 mmol) were successively added. The resulting mixture was then warmed and stirred at −78° C. for 4 h. After this time, the yellow suspension was cooled again to −95° C. before DMF (70 mL) was added. The final reaction mixture was warmed to −78° C. and stirred at that temperature for 1.5 h. The reaction mixture was poured into cold aqueous HCl (3N, 800 mL) and stirred for 5 min. Aqueous concentrated NH₄OH was added to adjust pH to 7.5. The aqueous layer was extracted three times with EtOAc. The combined organic layer was washed with aqueous NH₄Cl and brine, dried over anhydrous Nₐ2SO₄, filtered and concentrated. The crude material was further purified by a pad of silica gel by eluting with a gradient from 100% hexanes to 100% EtOAc and the product was crystallized in cold hexanes to yield the title compound as a pale yellow solid.

### Step 2 Methyl (2Z)-2-azido-3-(2-chloropyridin-3-yl)prop-2-enoate

A solution of 2-chloronicotinealdehyde (20.0 g, 139.9 mmol) and methyl azidoacetate (32.2 mL, 349.7 mmol) in MeOH (168 mL) was added to a solution of 25% NaOMe in MeOH (80 mL, 349 mmol) at −20 oC. The internal temperature was monitored and maintained at ˜−20° C. during the 30 min. addition. The resulting mixture was then stirred in an ice bath for several hours, followed by overnight in an ice bath in the cold room. The suspension was then poured onto a mixture of ice and NH₄Cl, and the slurry was filtered after 10 min. of stirring. The product was washed with cold H₂O and was then dried under vacuum. The crude material was dissolved in CH₂Cl₂ and MgSO₄ was added. The suspension was filtered through a pad of silica gel, washed with CH₂Cl₂. The filtrate was concentrated under reduced pressure and a beige precipitate (20 g) of the title product was obtained.

### Step 3 Methyl 4-chloro-1H-pyrrolo[3,2-c]pyridine-2-carboxylate

A solution of methyl (2Z)-2-azido-3-[2-chloropyridin-3-yl]prop-2-enoate (21 g, 88 mmol) in mesitylene (880 mL) was heated at reflux for a period of 1 h. The reaction mixture was cooled to room temperature then to 0° C., and the precipitate was filtered and washed with cold hexane. The material was stirred overnight in 1:20 EtOAc/hexane to give, after filtration, the title product as a pale yellow solid (13.2 g).

### Step 4 Methyl 1-chloro-8-oxo-7,8-dihydro-6H-pyrido[3,4-b]pyrrolizine-7-carboxylate

To a suspension of methyl 4-chloro-1H-pyrrolo[3,2-c]pyridine-2-carboxylate (12.5 g, 59 mmol) in THF (116 mL)-toluene (460 mL) were added a 1.0 M THF solution of potassium tert-butoxide (64 mL, 64 mmol) and methyl acrylate (55 mL, 611 mmol). The resulting mixture was heated at 100° C. for 18 h. After this time, the suspension was cooled to room temperature and it was poured into a mixture of saturated aqueous NH₄Cl (400 mL) and hexanes (400 mL). The solids were decanted, filtered and washed with H₂O and hexanes to provide the title compound.

### Step 5 1-Chloro-6,7-dihydro-8H-pyrido[3,4-b]pyrrolizin-8-one

To the compound of the previous step were added isopropanol (8.0 mL) and concentrated HCl (2.0 mL) with heating at 100° C. for 1 h. The reaction mixture was partitioned between EtOAc and Na₂CO₃. The organic phase was separated, evaporated to provide the title compound.

### Step 6 1-Isopropenyl-6,7-dihydro-8H-pyrido[3,4-b]pyrrolizin-8-one

To a mixture of 1-chloro-6,7-dihydro-8H-pyrido[3,4-b]pyrrolizin-8-one (5.0 g, 24.3 mmol), tris (dibenzylidene acetone)dipalladium (0) (1.0 g, 1.09 mmol) and triphenylarsine (2.70 g, 8.82 mmol) in DMF (100 mL) was added tributylisopropenyl stannane (9.60 g, 29.00 mmol). The resulting mixture was degassed and heated at 78° C. for a period of 18 h. The solvent was evaporated under reduced pressure. CH₂Cl₂ and celite were added to the resulting mixture which was then filtered over celite. The title compound was purified by flash chromatography (50% to 100% EtOAc in Hexane).

### Step 7 Ethyl (2E)-(1-isopropenyl-6,7-dihydro-8H-pyrido[3,4-b]pyrrolizin-8-ylidene)ethanoate

To a solution of 1-isopropenyl-6,7-dihydro-8H-pyrido[3,4-b]pyrrolizin-8-one (0.60 g, 2.8 mmol) and triethyl phosphonoacetate (1.00 g, 4.46 mmol) in THF (24 mL) at −78° C. was added 80% NaH (0.12 g, 4.00 mmol), the reaction mixture was allowed to warm to 0° C., then to room temperature. The reaction mixture was poured onto saturated NH₄Cl and EtOAc. The organic phase was separated, dried over Na₂SO₄ and evaporated. The title compound was purified by flash chromatography (40% EtOAc in Hexane).

### Step 8 Ethyl (1-isopropyl-7,8-dihydro-6H-pyrido[3,4-b]pyrrolizin-8-yl)acetate

To a solution of ethyl (2E)-(1-isopropenyl-6,7-dihydro-8H-pyrido[3,4-b]pyrrolizin-8-ylidene)ethanoate (0.40 g, 1.4 mmol) in MeOH (20 mL) was added Pd(OH)₂ (0.20 g). The mixture was stirred under 1 atm of H₂ for 3 h. The mixture was filtered over celite and evaporated to provide the title compound.

### Step 9 Ethyl {9-[(3,4-dichlorophenyl)thio]-1-isopropyl-7,8-dihydro-6H-pyrido[3,4-b]pyrrolizin-8-yl}acetate

To a solution of bis(3,4-dichlorophenyl)disulfide (0.24 g, 0.67 mmol) in CH₂Cl₂ (5.6 mL) was added SO₂Cl₂ (0.036 mL). The resulting yellow mixture was stirred at room temperature for 1 h. This solution was added to a solution of ethyl (1-isopropyl-7,8-dihydro-6H-pyrido[3,4-b]pyrrolizin-8-yL) acetate (0.15 g, 0.52 mmol) in DMF (5.6 mL) at 0° C. After 1.5 h at 0° C., the reaction mixture was poured over saturated NaHCO₃ and EtOAc. The organic phase was separated, dried over Na₂SO₄, filtered and evaporated. The title compound was purified by flash chromatography (30% to 40% EtOAc in Hexane).

### Step 10 {9-[(3,4-Dichlorophenyl)thio]-1-isopropyl-7,8-dihydro-6H-pyrido[3,4-b]pyrrolizin-8-yl}acetic acid

To a solution of ethyl {9-[(3,4-dichlorophenyl)thio]-1-isopropyl-7,8-dihydro-6H-pyrido[3,4-b]pyrrolizin-8yl}acetate (0.23 g, 0.50 mmol) in THF (5 mL and MeOH (2.5 mL) was added 1.0 M NaOH (1.5 mL, 1.5 mmol). After stirring 18 h at RT, HOAc (0.25 mL) was added and the solvent was evaporated. The residue was taken up in EtOAc/H₂O, and the organic layer was washed with H₂O and brine. After drying (Na₂SO₄), the solution was filtered and evaporated. The residue was stirred with 1:1 EtOAc:hex to give, after filtration, the title compound as a white solid.

¹H NMR (MeOH-d₄) δ 1.14-1.26 (m, 6H), 2.47-2.56 (m, 1H), 2.56-2.64 (m, 1H), 2.94-3.05 (m, 2H), 3.81-3.89 (m, 1H), 4.22-4.30 (m, 1H), 4.33-4.44 (m, 2H), 6.93-6.99 (m, 1H), 7.14-7.19 (m, 1H), 7.33-7.39 (m, 1H), 7.54-7.59 (m, 1H), 8.16-8.21 (m, 1H).

The product of Step 10 was converted to its methyl ester using CH₂N₂, and the ester was subjected to HPLC separation on chiral stationary phase (chiralcel OD column 2×25 cm), eluting with 12% 2-propanol in hexane at a flow rate of 6 mL/min. Enantiomer A (less polar) has a retention time of 31.9 min and Enantiomer B (more polar) has a retention time of 35.5 min. Both A and B were hydrolyzed as in Ex. 17 Step 10 to give enantiomers A and B of the title compound.

### DP Example 22

### ((1R)-6-Fluoro-8-(methylsulfonyl)-9-{(1S)-1-[4-(trifluoromethyl)phenyl]ethyl }-2,3,4,9-tetrahydro-1H-carbazol-1-yl)acetic acid (Compound AJ)

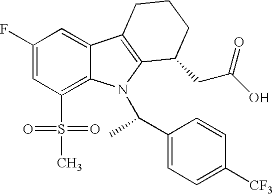

### Step 1: 2-(2-Bromo-4-fluorophenyl)hydrazinium chloride

To a suspension of 2-bromo-4-fluoroaniline in concentrated HCl (1.5M) at −10° C. was slowly added a 10.0M aqueous solution of NaNO₂ (1.1 eq). The mixture was stirred at 0° C. for 2.5 hrs. A cold (−30° C.) solution of SnCl₂ (3.8M) in concentrated HCl was then slowly added while maintaining the internal temperature below 10° C. The resulting mixture was stirred mechanically for 20 min at 10° C., then at room temperature for 1 hr. The thick slurry was filtered and the solid was air dried overnight. The solid was resuspended in cold HCl and filtered again. The dried material was suspended in Et₂O, stirred for 10 min, filtered and air dried overnight to give the title compound as a beige solid.

### Step 2: (+/−)-Ethyl (8-bromo-6-fluoro-2,3,4,9-tetrahydro-1H-carbazol-1-yl)acetate

To a suspension of the compound of Step 1 (1 eq) in AcOH (0.5M) was added ethyl (2-oxocyclohexyl)acetate (1 eq). The mixture was stirred at reflux for 16 hrs, cooled and AcOH was removed by evaporation under reduced pressure. The residue was diluted with EtOAc and washed with water and saturated aqueous NaHCO₃. The organic layer was dried over Na₂SO₄ and concentrated. The residue was then purified on a pad of silica gel, eluting with toluene. The filtrate was concentrated and stirred in hexanes to give, after filtration, the title compound as a white solid. MS (+APCI) m/z 354.2 (M+H)⁺.

### Step 3: (+/−)-Ethyl [6-fluoro-8-(methylsulfonyl)-2,3,4,9-tetrahydro-1H-carbazol-1-yl]-acetate

To a solution of the compound of Step 2 (1 eq) in anhydrous DMSO (0.28M) were added sodium methanesulphinate (3 eq) and copper iodide (3 eq). N₂ was bubbled into the mixture for 5 min and the reaction was then stirred at 100° C. under N₂ atmosphere. After 12 hrs, more sodium methanesulphinate (2 eq) and copper iodide (2 eq) were added. The mixture was stirred for a further 12 hrs at 100° C., cooled, diluted with EtOAc and 1N HCl was added to acidify the mixture. The suspension was stirred for 30 min and filtered through celite. The filtrate was washed with water, dried over Na₂SO₄ and concentrated. The residue was filtered through a pad of silica gel, eluting first with toluene to remove the non-polar impurities and then with a 2:1 mixture of hexanes/EtOAc to elute the desired product. The filtrate from the elution with the mixture of hexanes/EtOAc was concentrated to give the title compound as a pale yellow solid. MS (-APCI) m/z 352.1 (M−H)

### Step 4: Ethyl [(1R)-6-fluoro-8-(methylsulfonyl)-2,3,4,9-tetrahydro-1H-carbazol-1-yl]acetate

The racemic mixture from step 3 was resolved by preparative HPLC on a chiralpak AD preparative column eluted with a mixture of 15% iPrOH in hexane. The more polar enantiomer (longer retention time) was identified as the title compound based on the activity of the final product.

### Step 5: Ethyl [(1R)-9-[(1S)-1-(4-chlorophenyl)ethyl]-6-fluoro-8-(methylsulfonyl)-2,3,4,9-tetrahydro-1H-carbazol-1-yl]acetate

To a solution of the compound of Step 4 (1 eq), triphenylphosphine (1.5 eq) and (1R)-1-(4-chlorophenyl)ethanol (1.5 eq, prepared following the general procedure described in Reference Example 1) in THF (0.175M) was added a solution of di-tert-butyl azodicarboxylate (2.1 M in THF, 1.5 eq) over a 10 min period. The mixture was stirred at room temperature for 2 hr and concentrated. The residue was purified by silica gel flash chromatography, eluting with 7% EtOAc in toluene to give the desired product (˜90% pure) which was used as such for the next reaction.

### Step 6: [(1R)-9-[(1S)-1-(4-Chlorophenyl)ethyl]-6-fluoro-8-(methylsulfonyl)-2,3,4,9-tetrahydro-1H-carbazol-1-yl]acetic acid and [(1S)-9-[(1S)-1-(4-chlorophenyl)ethyl]-6-fluoro-8-(methylsulfonyl)-2,3,4,9-tetrahydro-1H-carbazol-1-yl]acetic acid

To a solution of the compound of Step 5 in a 2:1 mixture of THF and methanol (0.1M) was added 1N aqueous LiOH (3 eq). The mixture was stirred at room temperature for 2 hr, AcOH was added and the solvent was removed by evaporation. The residue was taken up in EtOAc/H₂O and the organic layer was washed with brine, dried over Na₂SO₄, filtered and concentrated. The residue was swished in 30% EtOAc in hexane, and the product was suspended in diethyl ether and sonicated for 45 min, filtered, and dried under high vacuum at 50° C. for 24 hr to give the title compound as a white solid. MS (-APCI) m/z 462.1 (M−H)

Alternatively (+/−) ethyl [6-fluoro-8-(methylsulfonyl)-2,3,4,9-tetrahydro-1H-carbazol-1-yl]acetate was used for the alkylation reaction in step 5 to give a mixture of 2 diastereomers: ethyl [(1R)-9-[(1S)-1-(4-chlorophenyl)ethyl]-6-fluoro-8-(methylsulfonyl)-2,3,4,9-tetrahydro-1H-carbazol-1-yl]acetate and ethyl [(1S)-9-[(1S)-1-(4-chlorophenyl)ethyl]-6-fluoro-8-(methylsulfonyl)-2,3,4,9-tetrahydro-1H-carbazol-1-yl]acetate. The diastereomeric mixture was resolved by selective hydrolysis using the following procedure to give the desired [(1R)-9-[(1S)-1-(4-chlorophenyl)ethyl]-6-fluoro-8-(methylsulfonyl)-2,3,4,9-tetrahydro-1H-carbazol-1-yl]acetic acid.

### RESOLUTION

The diastereomeric mixture of ethyl [(1R)-9-[(1S)-1-(4-chlorophenyl)ethyl]-6-fluoro-8-(methylsulfonyl)-2,3,4,9-tetrahydro-1H-carbazol-1-yl]acetate and ethyl [(1S)-9-[(1S)-1-(4-chlorophenyl)ethyl]-6-fluoro-8-(methylsulfonyl)-2,3,4,9-tetrahydro-1H-carbazol-1-yl]acetate (1 eq) was dissolved in a 3.5/1 mixture of THF/MeOH (0.25M) and cooled at 0° C. Aqueous LiOH 1N (1 eq) was slowly added and the mixture was stirred at 0° C. for 12 h or until almost complete hydrolysis of ethyl [(1R)-9-[(1S)-1-(4-chlorophenyl)ethyl]-6-fluoro-8-(methylsulfonyl)-2,3,4,9-tetrahydro-1H-carbazol-1-yl]acetate, the other diastereomer was only slightly hydrolyzed under these conditions. AcOH was added and the solvent was removed by evaporation. The residue was taken up in EtOAc/H₂O and the organic layer was washed with brine, dried over Na₂SO₄, filtered and concentrated. Ethyl [(1S)-9-[(1S)-1-(4-chlorophenyl)ethyl]-6-fluoro-8-(methylsulfonyl)-2,3,4,9-tetrahydro-1H-carbazol-1-yl]acetate and [(1R)-9-[(1S)-1-(4-chlorophenyl)ethyl]-6-fluoro-8-(methylsulfonyl)-2,3,4,9-tetrahydro-1H-carbazol-1-yl]acetic acid were separated by flash chromatography eluting with 40% EtOAc in hexanes containing 1% AcOH to give the desired [(1R)-9-[(1S)-1-(4-chlorophenyl)ethyl]-6-fluoro-8-(methylsulfonyl)-2,3,4,9-tetrahydro-1H-carbazol-1-yl]acetic acid with de>90% which was swished in 30% EtOAc in hexane to give the desired compound as a white solid with de>95%.

### Step 7: Methyl [(1R)-6-fluoro-8-(methylsulfonyl)-2,3,4,9-tetrahydro-1H-carbazol-1-yl]acetate

To a solution of [(1R)-9-[(1S)-1-(4-chlorophenyl)ethyl]-6-fluoro-8-(methylsulfonyl)-2,3,4,9-tetrahydro-1H-carbazol-1-yl]acetic acid ([α]_{D}=−226° in MeOH) in MeOH (0.1M) was added 10% palladium on carbon (10% wt/wt). A stream of N₂ was bubbled through the mixture for 5 min. The reaction was stirred at rt under H₂ atmosphere(balloon) for 24 hrs and filtered through a celite pad eluted with CH₂Cl₂. The solvents were removed by evaporation under reduced pressure and the residue was swished in MeOH to give the compound methyl [(1R)-6-fluoro-8-(methylsulfonyl)-2,3,4,9-tetrahydro-1H-carbazol-1-yl]acetate.

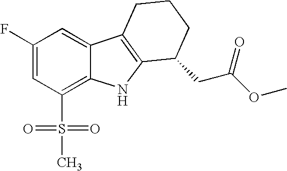

### Step 8: ((1R)-6-Fluoro-8-(methylsulfonyl)-9-{(1S)-1-[4-(trifluoromethyl)phenyl]ethyl}-2,3,4,9-tetrahydro-1H-carbazol-1-yl)acetic acid (Compound AJ)

To a solution of the compound of step 7 (1 eq), triphenylphosphine (1.5 eq) and (1R)-1-[4-(trifluoromethyl)phenyl]ethanol (1.5 eq) in THF (0.2M) was added a solution of di-tert-butyl azodicarboxylate (1M in THF, 1.5 eq) over a 20 min period. The mixture was stirred at room temperature for 2 hr and concentrated. The residue was purified by silica gel flash chromatography eluted with 10% EtOAc in toluene to give methyl ((1R)-6-fluoro-8-(methylsulfonyl)-9-{(1S)-1-[4-(trifluoromethyl)phenyl]ethyl}-2,3,4,9-tetrahydro-1H-carbazol-1-yl)acetate (˜90% pure) which was used as such for the next reaction.

To a solution of the above ester (1 eq) in a 3.5/1 mixture of THF/MeOH (0.25M) at 0° C. was slowly added aqueous LiOH 1N (1 eq) and the mixture was stirred at 0° C. for 16 h or until almost complete hydrolysis of the ester; under these conditions, the other minor diastereomer has a much slower rate of hydrolysis. AcOH was added and the solvent was removed in vacuo. The residue was taken up in EtOAc/H₂O and the organic layer was washed with brine, dried over Na₂SO₄, filtered and concentrated. To remove the unreacted methyl ester, the residue was filtered through a pad of silica gel eluting first with 10% EtOAc/toluene and then with 60% EtOAc/toluene containing 1% of AcOH. The residue was swished in 30% EtOAc/hexane and dried under high vacuum at 50° C. for 16 hr to give the title compound as a white solid with de and ee>95% (checked by chiral HPLC). MS (-APCI) m/z 496.0 (M−H)⁻. [α]_{D}=−181° in MeOH

Biological Assays

The activity of the compounds of the present invention regarding niacin receptor affinity and function can be evaluated using the following assays:

³H-Niacin binding assay:

1. Membrane: Membrane preps are stored in liquid nitrogen in:
   20 mM HEPES, pH 7.4
   0.1 mM EDTA
   Thaw receptor membranes quickly and place on ice. Resuspend by pipetting up and down vigorously, pool all tubes, and mix well. Use clean human at 15 ug/well, clean mouse at 10 ug/well, dirty preps at 30 ug/well.
   1a. (human): Dilute in Binding Buffer.
   1b. (human+4% serum): Add 5.7% of 100% human serum stock (stored at −20C.) for a final concentration of 4%. Dilute in Binding Buffer.
   1c. (mouse): Dilute in Binding Buffer.
2. Wash buffer and dilution buffer: Make 10 liters of ice-cold Binding Buffer:
   20 mM HEPES, pH 7.4
   1 mM MgCl₂
   0.01% CHAPS (w/v)
   use molecular grade or ddH₂O water
3. [5, 6-³ H]-nicotinic acid: American Radiolabeled Chemicals, Inc. (cat #ART-689). Stock is ˜50 Ci/mmol, 1 mCi/ml, 1 ml total in ethanol→20 μM
   Make an intermediate ³H-niacin working solution containing 7.5% EtOH and 0.25 μM tracer.
   40 μl of this will be diluted into 200 μl total in each well→1.5% EtOH, 50 nM tracer final.
4. Unlabeled nicotinic acid:
   Make 100 mM, 10 mM, and 80 uM stocks; store at −20C. Dilute in DMSO.
5. Preparing Plates:
1) Aliquot manually into plates. All compounds are tested in duplicate. 10 mM unlabeled nicotinic acid must be included as a sample compound in each experiment.
2) Dilute the 10 mM compounds across the plate in 1:5 dilutions (8 ul:40 ul).
3) Add 195 μl binding buffer to all wells of Intermediate Plates to create working solutions (250 μM→0). There will be one Intermediate Plate for each Drug Plate.

2) Transfer 5 μl from Drug Plate to the Intermediate Plate. Mix 4-5 times.
6. Procedure:
1) Add 140 μl of appropriate diluted 19CD membrane to every well. There will be three plates for each drug plate: one human, one human+serum, one mouse.
2) Add 20 μl of compound from the appropriate intermediate plate
3) Add 40 μl of 0.25 μM ³H-nicotinic acid to all wells.
4) Seal plates, cover with aluminum foil, and shake at RT for 3-4 hours, speed 2, titer plate shaker.
5) Filter and wash with 8×200 μl ice-cold binding buffer. Be sure to rinse the apparatus with >1 liter of water after last plate.
6) Air dry overnight in hood (prop plate up so that air can flow through).
7) Seal the back of the plate
8) Add 40 μL Microscint-20 to each well.
9) Seal tops with sealer.
10) Count in Packard Topcount scintillation counter.
11) Upload data to calculation program, and also plot raw counts in Prism, determining that the graphs generated, and the IC₅₀ values agree.

The compounds of the invention generally have an IC₅₀ in the ³H-nicotinic acid binding competition assay within the range of 1 nM to about 25 μM.

³⁵S-GTPγS binding assay:

Membranes prepared from Chinese Hamster Ovary (CHO)-K1 cells stably expressing the niacin receptor or vector control (7 μg/assay) were diluted in assay buffer (100 mM HEPES, 100 mM NaCl and 10 mM MgCl₂, pH 7.4) in Wallac Scintistrip plates and pre-incubated with test compounds diluted in assay buffer containing 40 μM GDP (final [GDP] was 10 μM) for ˜10 minutes before addition of ³⁵S-GTPγS to 0.3 nM. To avoid potential compound precipitation, all compounds were first prepared in 100% DMSO and then diluted with assay buffer resulting in a final concentration of 3% DMSO in the assay. Binding was allowed to proceed for one hour before centrifuging the plates at 4000 rpm for 15 minutes at room temperature and subsequent counting in a TopCount scintillation counter. Non-linear regression analysis of the binding curves was performed in GraphPad Prism.
   Membrane Preparation
   Materials:
CHO-K1 cell culture medium: F-12 Kaighn's Modified Cell Culture Medium with 10% FBS, 2 mM L-Glutamine, 1 mM Sodium Pyruvate and 400 μg/ml G418
Membrane Scrape Buffer: 20 mM HEPES
   10 mM EDTA, pH 7.4
Membrane Wash Buffer: 20 mM HEPES
   0.1 mM EDTA, pH 7.4
Protease Inhibitor Cocktail: P-8340, (Sigma, St. Louis, Mo.)
   Procedure:

(Keep everything on ice throughout prep; buffers and plates of cells)
Aspirate cell culture media off the 15 cm² plates, rinse with 5 mL cold PBS and aspirate.
Add 5 ml Membrane Scrape Buffer and scrape cells. Transfer scrape into 50 mL centrifuge tube. Add 50 uL Protease Inhibitor Cocktail.
Spin at 20,000 rpm for 17 minutes at 4° C.
Aspirate off the supernatant and resuspend pellet in 30 mL Membrane Wash Buffer. Add 50 uL Protease Inhibitor Cocktail.
Spin at 20,000 rpm for 17 minutes at 4° C.
Aspirate the supernatant off the membrane pellet. The pellet may be frozen at −80° C. for later use or it can be used immediately.
   Assay
   Materials:
Guanosine 5′-diphosphate sodium salt (GDP, Sigma-Aldrich Catalog #87127)
Guanosine 5′-[γ³⁵S] thiotriphosphate, triethylammonium salt ([³⁵S]GTPγS, Amersham Biosciences Catalog #SJ1320, ˜1000 Ci/mmol)
96 well Scintiplates (Perkin-Elmer #1450-501)
Binding Buffer: 20 mM HEPES, pH 7.4
   100 mM NaCl
   10 mM MgCl₂
GDP Buffer: binding buffer plus GDP, ranging from 0.4 to 40 μM, make fresh before assay
   Procedure:
(total assay volume=100λ/well)
25 μL GDP buffer with or without compounds (final GDP 10 μM-so use 40 μM stock)
50 μL membrane in binding buffer (0.4 mg protein/mL)
25 μL [³⁵S]GTPγS in binding buffer. This is made by adding 5 μl [³⁵S]GTPγS stock into 10 mL binding buffer (This buffer has no GDP)
   Thaw compound plates to be screened (daughter plates with 5 μL compound @ 2 mM in 100% DMSO)
   Dilute the 2 mM compounds 1:50 with 245 μL GDP buffer to 40 μM in 2% DMSO. (Note: the concentration of GDP in the GDP buffer depends on the receptor and should be optimized to obtain maximal signal to noise; 40 μM).
   Thaw frozen membrane pellet on ice. (Note: they are really membranes at this point, the cells were broken in the hypotonic buffer without any salt during the membrane prep step, and most cellular proteins were washed away)
   Homogenize membranes briefly (few seconds—don't allow the membranes to warm up, so keep on ice between bursts of homogenization) until in suspension using a POLYTRON PT3100 (probe PT-DA 3007/2 at setting of 7000 rpm). Determine the membrane protein concentration by Bradford assay. Dilute membrane to a protein concentrations of 0.40 mg/ml in Binding Buffer. (Note: the final assay concentration is 20 μg/well).
   Add 25 μL compounds in GDP buffer per well to Scintiplate.
   Add 50 μL of membranes per well to Scintiplate.
   Pre-incubate for 5-10 minutes at room temperature. (cover plates with foil since compounds may be light sensitive)
   Add 25 μL of diluted [³⁵S]GTPγS. Incubate on shaker (Lab-Line model #1314, shake at setting of 4) for 60 minutes at room temperature. Cover the plates with foil since some compounds might be light sensitive.
   Assay is stopped by spinning plates sealed with plate covers at 2500 rpm for 20 minutes at 22° C.
   Read on TopCount NXT scintillation counter-35S protocol.

The compounds of the invention generally have an EC₅₀ in the functional in vitro GTPγS binding assay within the range of about less than 1 μM to as high as about 100 μM.

Flushing via Laser Doppler

Male C57B16 mice (˜25 g) are anesthetized using 10 mg/ml/kg Nembutal sodium. When antagonists are to be administered they are co-injected with the Nembutal anesthesia. After ten minutes the animal is placed under the laser and the ear is folded back to expose the ventral side. The laser is positioned in the center of the ear and focused to an intensity of 8.4-9.0 V (with is generally ˜4.5 cm above the ear). Data acquisition is initiated with a 15 by 15 image format, auto interval, 60 images and a 20 sec time delay with a medium resolution. Test compounds are administered following the 10th image via injection into the peritoneal space. Images 1-10 are considered the animal's baseline and data is normalized to an average of the baseline mean intensities.

Materials and Methods—Laser Doppler Pirimed PimII; Niacin (Sigma); Nembutal (Abbott labs).

Certain compounds of the invention do not exhibit measurable in vivo vasodilation in this murine flushing model at doses up to 100 mg/kg or 300 mg/kg.

All patents, patent applications and publications that are cited herein are hereby incorporated by reference in their entirety. While certain preferred embodiments have been described herein in detail, numerous alternative embodiments are seen as falling within the scope of the invention.

What is claimed is:

1. A compound in accordance with formula I: 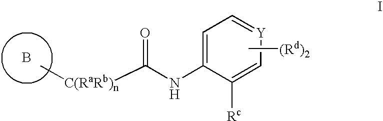
or a pharmaceutically acceptable salt or solvate thereof, wherein:
Y represents C or N;
R^{a} and R^{b} are independently H, C₁₋₃alkyl, haloC₁₋₃alkyl, OC₁₋₃alkyl, haloC₁₋₃alkoxy, OH or F;
R^{c} represents —CO₂H, 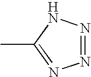
or —C(O)NHSO₂R^{1a};
R^{1a} represents C₁₋₄alkyl or phenyl, said C₁₋₄alkyl or phenyl being optionally substituted with 1-3 substituent groups, 1-3 of which are selected from halo and C₁₋₃alkyl, and 1-2 of which are selected from the group consisting of: OC₁₋₃alkyl, haloC₁₋₃alkyl, haloC₁₋₃alkoxy, OH, NH₂ and NHC₁₋₃alkyl;
each R^{d} independently represents H, halo, methyl, or methyl substituted by 1-3 halo groups;
ring B represents a 10 membered bicyclic aryl, a 9-10 membered bicyclic heteroaryl or a 12-13 membered tricyclic heteroaryl group, 0-1 members of which are O or S and 0-4 members of which are N; said bicyclic aryl or heteroaryl group being optionally substituted with 1-3 groups, 1-3 of which are halo groups and 1-2 of which are selected from the group consisting of:
a) OH; CO₂H; CN; NH₂; S(O)₀₋₂R^{1a};
b) C₁₋₆ alkyl and OC₁₋₆alkyl, said group being optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄allyl, N(C₁₋₄alkyl)₂, Hetcy, CN;
c) Hetcy, NHC₁₋₄alkyl and N(C₁₋₄alkyl)₂, the alkyl portions of which are optionally substituted as set forth in (b) above;
d) Aryl, HAR, C(O)Aryl and C(O) HAR, the Aryl and HAR portions being optionally substituted as set forth in (b) above;
e) C(O)C₁₋₄alkyl and CO₂C₁₋₄alkyl, the alkyl portions of which are optionally substituted as set forth in (b) above; and
f) C(O)NH₂, C(O)NHC₁₋₄alkyl, C(O)N(C₁₋₄alkyl)₂, C(O)NHOC₁₋₄alkyl, C(O)N(C₁₋₄alkyl)(OC₁₋₄alkyl) and C(O)Hetcy, the alkyl portions of which are optionally substituted as set forth in (b) above;
g) NR′C(O)R″, NR′SO₂R″, NR′CO₂R″ and NR′C(O)NR″R′″ wherein:
R′ represents H, C₁₋₃alkyl or haloC₁₋₃alkyl,
R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, CN, ethynyl, Hetcy, Aryl and HAR,
said Hetcy, Aryl and HAR being further optionally substituted with 1-3 halo, C₁₋₄alkyl, C₁₋₄alkoxy, haloC₁₋₄alkyl and haloC₁₋₄alkoxy groups;
(b) Hetcy, Aryl or HAR, said Hetcy, Aryl and HAR being further optionally substituted with 1-3 halo, C₁₋₄alkyl, C₁₋₄alkoxy, haloC₁₋₄alkyl and haloC₁₋₄alkoxy groups;
and R′″ representing H or R″;
n represents an integer of from 1 to 4, such that (i) when (CR^{a}R^{b}) represents 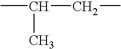
and ring B represents a bicyclic aryl group, said bicyclic aryl group is substituted;
and (ii) when ring B represents a 9-membered heteroaryl group containing one heteroatom, said heteroatom is S or O.

2. A compound in accordance with claim 1 wherein ring B represents naphthyl or a bicyclic 9-10 membered heteroaryl group containing 1-2 heteroatoms, 0-1 of which is O or S, and 1-2 of which are nitrogen.

3. A compound in accordance with claim 2 wherein ring B represents naphthyl, quinolinyl, isoquinolinyl or benzothiazolyl.

4. A compound in accordance with claim 3 wherein ring B represents 1- or 2-naphthyl, 2-, 6- or 7-quinolinyl, 5-, 6- or 7-isoquinolinyl, or 5- or 6-benzothiazolyl.

5. A compound in accordance with claim 4 wherein B represents naphthyl or quinolinyl.

6. A compound in accordance with claim 5 wherein B represents naphthyl.

7. A compound in accordance with claim 5 wherein B represents quinolinyl.

8. A compound in accordance with claim 5 wherein B represents isoquinolinyl.

9. A compound in accordance with claim 1 wherein:
Ring B is selected from naphthyl, quinolinyl, isoquinolinyl and benzothiazolyl, optionally substituted with 1-3 groups, 1-3 of which are halo groups selected from Cl and F, and 1-2 groups are selected from:
a) OH; CO₂H; CN; NH₂;
b) C₁₋₄ alkyl and OC₁₋₄alkyl, said group being optionally substituted with 1-3 groups, 1-3 of which are halo selected from Cl and F, and 1 of which is selected from: OH, CO₂H, CO₂C₁₋₂alkyl, CO₂C₁₋₂haloalkyl wherein halo is selected from Cl and F, OCO₂C₁₋₄alkyl, NH₂, NHC₁₋₄alkyl, N(C₁₋₄alkyl)₂, Hetcy and CN;
c) Hetcy, NHC₁₋₄alkyl and N(C₁₋₄alkyl)₂, the alkyl portions of which are optionally substituted as set forth in (b) above;
d) C(O)NH₂, C(O)NHC₁₋₄alkyl and C(O)N(C₁₋₂alkyl)₂, the alkyl portions of which are optionally substituted as set forth in (b) above;
e) NR′C(O)R″, NR′SO₂R″, NR′CO₂R″ and NR′C(O)NR″R′″ wherein:
R′ represents H, C₁₋₃alkyl or haloC₁₋₃alkyl wherein halo is selected from Cl and F,
R″ represents (a) C₁₋₈alkyl optionally substituted with 1-4 groups, 0-4 of which are halo selected from Cl and F, and 0-1 of which are selected from the group consisting of: OC₁₋₄alkyl, OH, CO₂H, CO₂C₁₋₄alkyl, CO₂C₁₋₄haloalkyl, OCO₂C₁₋₄alkyl, NH₂, NHC₁₋₄alkyl, N(C₁₋₂alkyl)₂, CN, ethynyl, Hetcy, Aryl and HAR,
said Hetcy, Aryl and HAR being further optionally substituted with 1-3 halo groups, C₁₋₄alkyl, C₁₋₄alkoxy, haloC₁₋₄alkyl and haloC₁₋₄alkoxy groups, the halo and halo portions of which are selected from Cl and F;
(b) Hetcy, Aryl or HAR, said Hetcy, Aryl and HAR being further optionally substituted with 1-3 halo, C₁₋₄alkyl, C₁₋₄alkoxy, haloC₁₋₄alkyl and haloC₁₋₄alkoxy groups, the halo and halo portions of which are selected from Cl and F;
and R′″ representing H or R″.

10. A compound in accordance with claim 9 wherein:
Ring B is naphthyl optionally substituted with 1-2 halo groups selected from Cl and F, and 0-1 group selected from:
a) OH;
b) C₁₋₄ alkyl and OC₁₋₄alkyl, said group being optionally substituted with 1-3 groups, 1-3 of which are halo selected from Cl and F;
c) NR′C(O)R″, NR′SO₂R″, NR′CO₂R″ and NR′C(O)NR″R′″ wherein:
R′ represents H, C₁₋₃alkyl or haloC₁₋₃alkyl wherein halo is selected from Cl and F,
R″ represents (a) C₁₋₈alkyl optionally substituted with 1-4 groups, 0-4 of which are halo selected from Cl and F, and 0-1 of which are selected from the group consisting of: OC₁₋₄alkyl, OH, CO₂H, CO₂C₁₋₄alkyl, CO₂C₁₋₄haloalkyl, OCO₂C₁₋₄alkyl, NH₂, NHC₁₋₄alkyl, N(C₁₋₂alkyl)₂, CN, ethynyl, Hetcy, Aryl and HAR,
said Hetcy, Aryl and HAR being further optionally substituted with 1-3 halo groups, C₁₋₄alkyl, C₁₋₄alkoxy, haloC₁₋₄alkyl and haloC₁₋₄alkoxy groups, the halo and halo portions of which are selected from Cl and F;
(b) Hetcy, Aryl or HAR, said Hetcy, Aryl and HAR being further optionally substituted with 1-3 halo, C₁₋₄alkyl, C₁₋₄alkoxy, haloC₁₋₄alkyl and haloC₁₋₄alkoxy groups, the halo and halo portions of which are selected from Cl and F;
and R′″ representing H or R″.

11. A compound in accordance with claim 1 wherein Y represents C.

12. A compound in accordance with claim 1 wherein Y represents N.

13. A compound in accordance with claim 1 wherein n represents 2, 3 or 4.

14. A compound in accordance with claim 13 wherein n represents an integer 2, 3 or 4, and one or both of R^{a} and R^{b} represents H or CH₃, and the remaining R^{a} and R^{b} groups, if any, represent H.

15. A compound in accordance with claim 1 wherein R^{c} represents CO₂H.

16. A compound in accordance with claim 1 wherein R^{c} represents tetrazolyl.

17. A compound in accordance with claim 1 wherein R^{d} represents H or halo.

18. A compound in accordance with claim 17 wherein R^{d} represents H.

19. A compound in accordance with claim 17 wherein R^{d} represents halo, selected from F.

20. A compound in accordance with claim 1, 2, 3, 4, 5, 6, 7, 8, 9, 10, 11, 12, 13, 15, 16, 17, 18 or 19 wherein:
one of R^{a} and R^{b} is selected from the group consisting of: C₁₋₃alkyl, haloC₁₋₃alkyl, OC₁₋₃alkyl, haloC₁₋₃alkoxy, OH and F, and the other is selected from the group consisting of: H, C₁₋₃alkyl, haloC₁₋₃alkyl, OC₁₋₃alkyl, haloC₁₋₃alkoxy, OH and F.

21. A compound in accordance with claim 20 wherein one of R^{a} and R^{b} is C₁₋₃alkyl.

22. A compound in accordance with claim 21 wherein one of R^{a} and R^{b} is methyl.

23. A compound in accordance with claim 1, 2, 3, 4, 5, 6, 7, 8, 9, 10, 11, 12, 13, 14, 15, 16, 20, 21 or 22 wherein at least one R^{d} group is selected from the group consisting of: halo, methyl and methyl substituted with 1-3 halo groups, and is located ortho or meta to R^{c}.

24. A compound in accordance with claim 1, 2, 3, 4, 5, 6, 7, 8, 9, 10, 11, 12, 13, 14, 15, 16, 17, 18, 19, 20, 21, 22 or 23 wherein ring B is substituted with from 1-3 groups, 1-3 of which are halo atoms, and 1-2 of which are selected from OH and NH₂.

25. A compound in accordance with claim 1, 11, 12, 13, 14, 15, 16, 17, 18, 19, 20, 21, 22, 23 or 24 wherein ring B represents a 12-13 membered tricyclic heteroaryl group, 0-1 members of which are O or S, and 0-4 of which are N, said group being optionally substituted with 1-3 groups, 1-3 of which are halo atoms and 1-2 of which are selected from the group consisting of:
a) OH; CO₂H; CN; NH₂; S(O)₀₋₂R^{1a};
b) C₁₋₆ alkyl and OC₁₋₆alkyl, said group being optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, Hetcy, CN;
c) Hetcy, NHC₁₋₄alkyl and N(C₁₋₄alkyl)₂, the alkyl portions of which are optionally substituted as set forth in (b) above;
d) Aryl, HAR, C(O)Aryl and C(O) HAR, the Aryl and HAR portions being optionally substituted as set forth in (b) above;
e) C(O)C₁₋₄alkyl and CO₂C₁₋₄alkyl, the alkyl portions of which are optionally substituted as set forth in (b) above; and
f) C(O)NH₂, C(O)NHC₁₋₄alkyl, C(O)N(C₁₋₄alkyl)₂, C(O)NHOC₁₋₄alkyl, C(O)N(C₁₋₄alkyl)(OC₁₋₄alkyl) and C(O)Hetcy, the alkyl portions of which are optionally substituted as set forth in (b) above;
g) NR′C(O)R″, NR′SO₂R″, NR′CO₂R″ and NR′C(O)NR″R′″ wherein:
R′ represents H, C₁₋₃alkyl or haloC₁₋₃alkyl,
R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, CN, ethynyl, Hetcy, Aryl and HAR,
said Hetcy, Aryl and HAR being further optionally substituted with 1-3 halo, C₁₋₄alkyl, C₁₋₄alkoxy, haloC₁₋₄alkyl and haloC₁₋₄alkoxy groups;
(b) Hetcy, Aryl or HAR, said Hetcy, Aryl and HAR being further optionally substituted with 1-3 halo, C₁₋₄alkyl, C₁₋₄alkoxy, haloC₁₋₄alkyl and haloC₁₋₄alkoxy groups;
and R′″ representing H or R″.

26. A compound in accordance with claim 25 wherein ring B represents a member selected from the group consisting of:

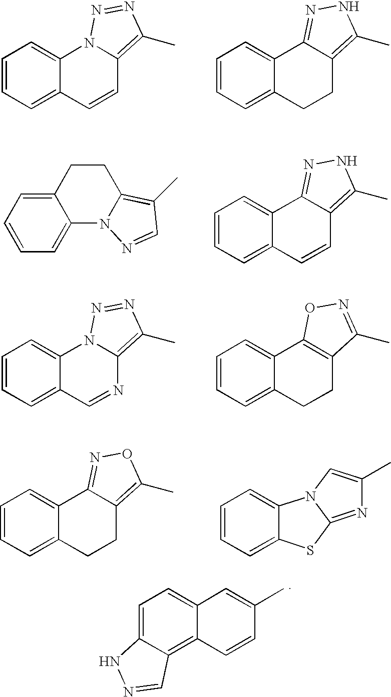

27. A compound in accordance with claim 1 selected from Table 1:
TABLE 1 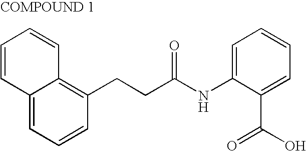 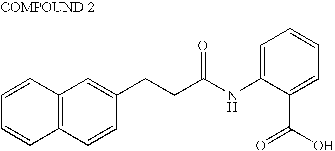 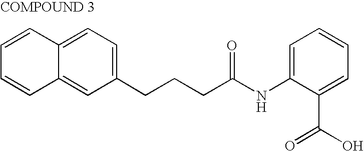 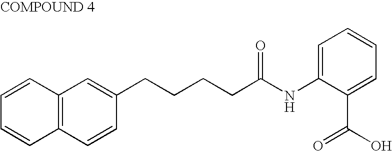 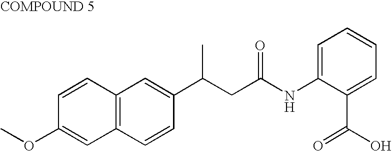 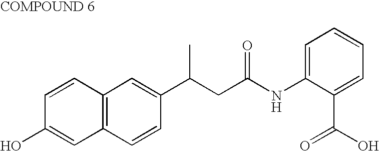 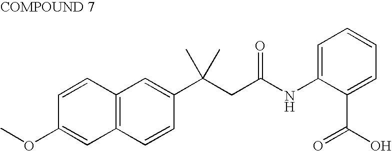 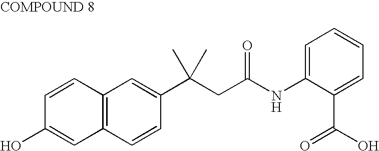 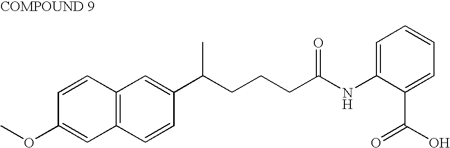 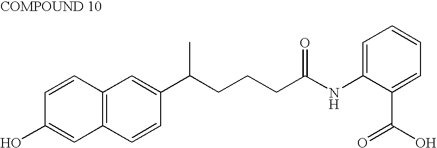 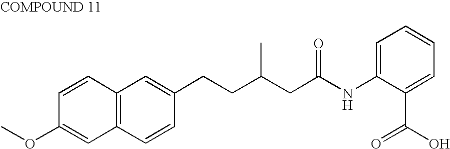 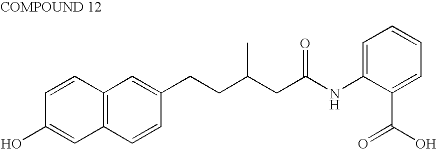 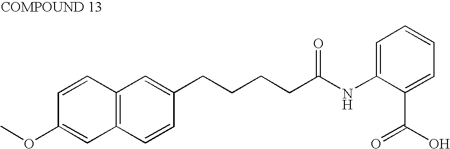 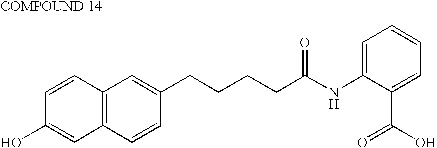 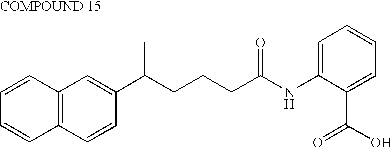 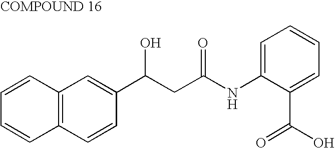 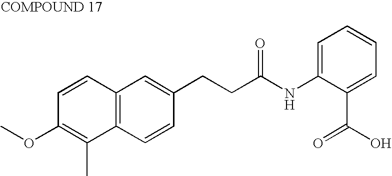 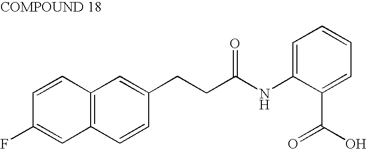 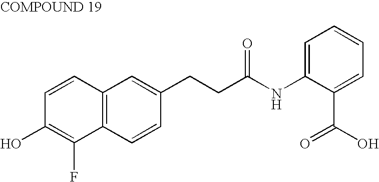 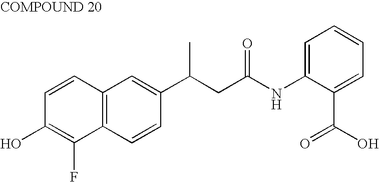 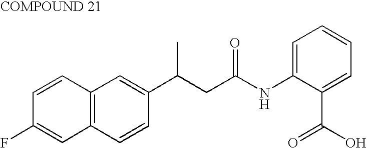 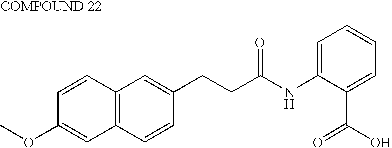 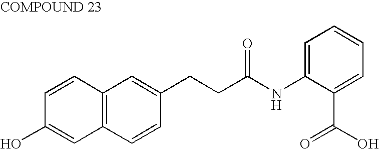 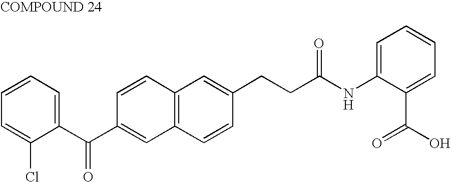 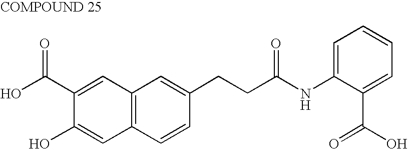 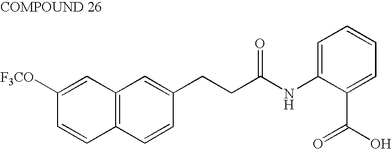 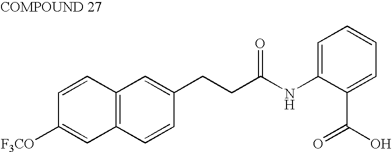 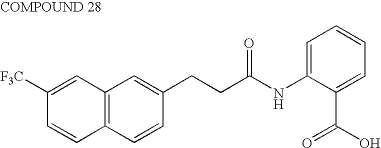 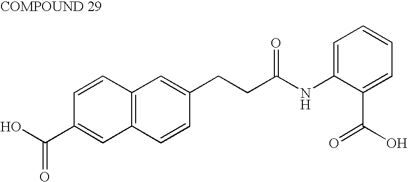 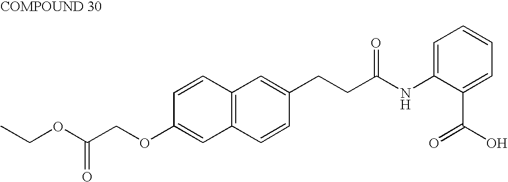 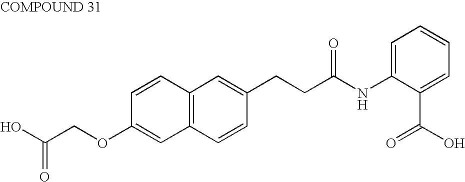 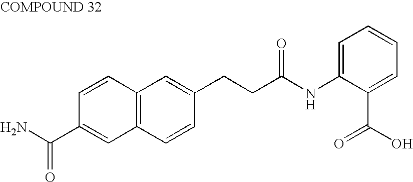 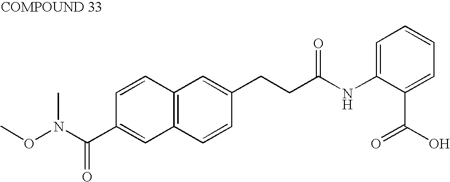 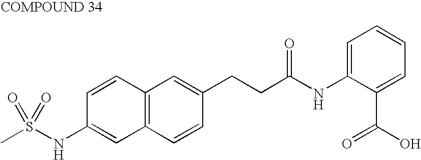 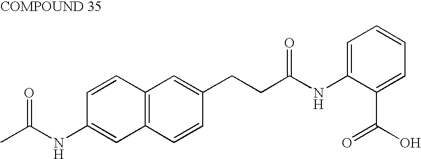 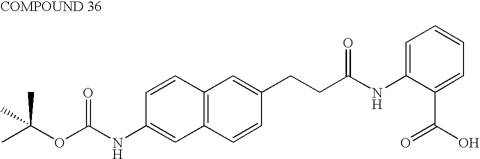 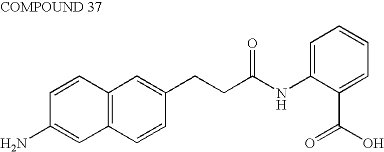 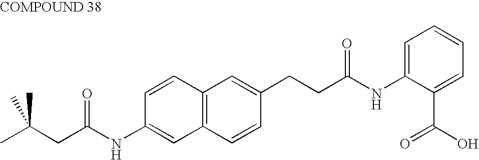 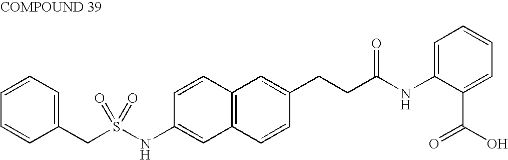 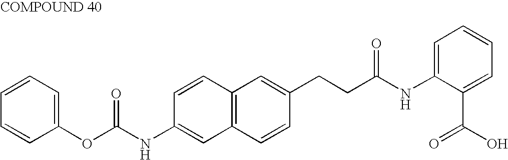 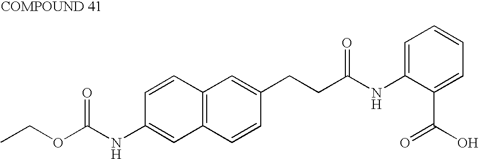 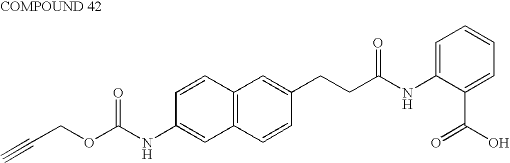 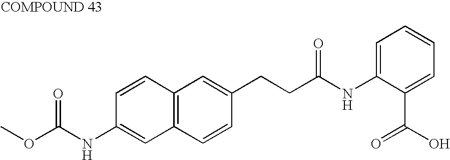 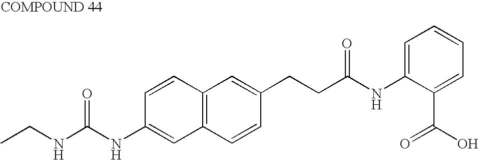 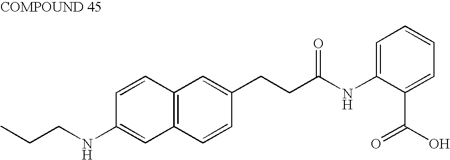 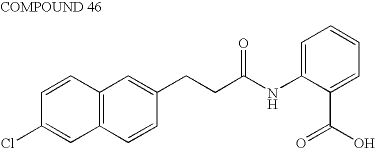 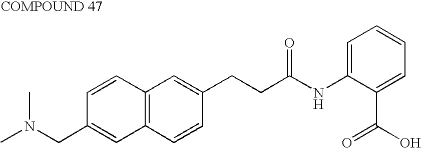 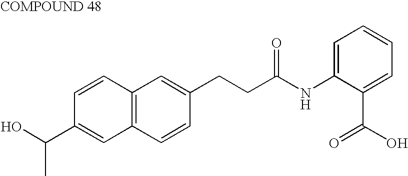 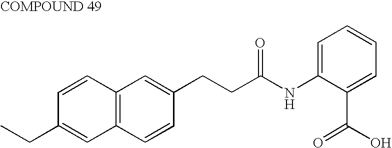 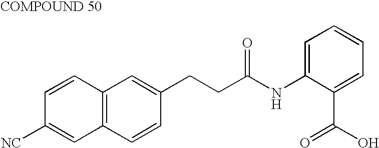 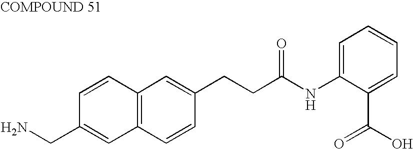 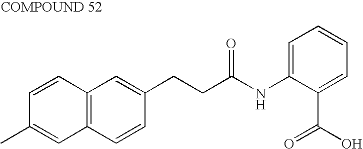 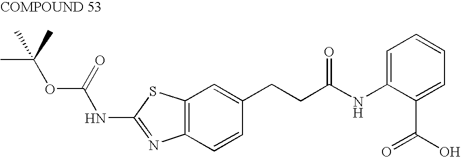 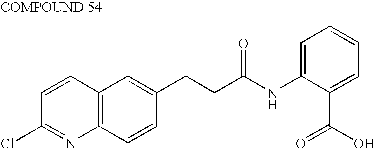 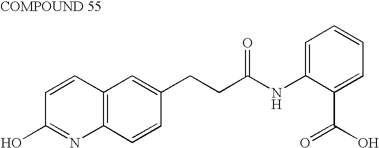 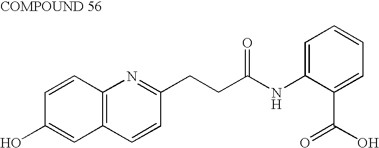 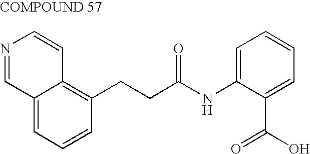 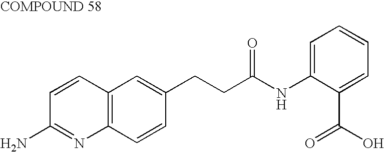 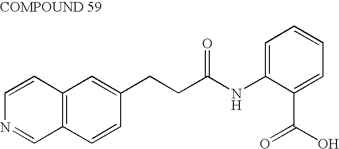 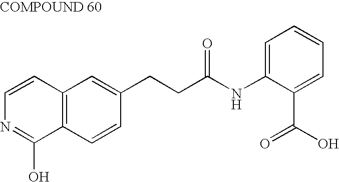 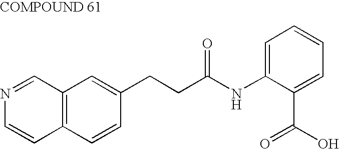 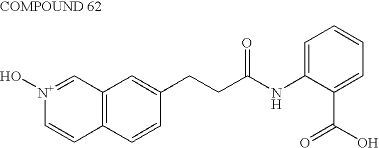 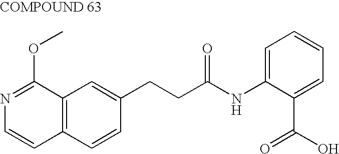 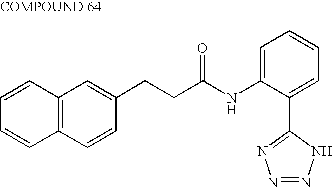 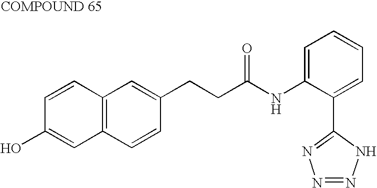 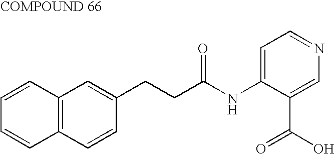 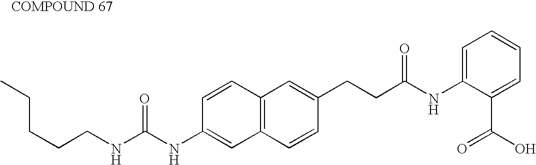 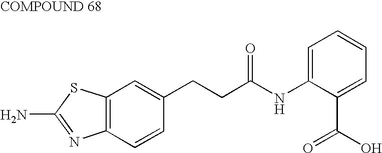 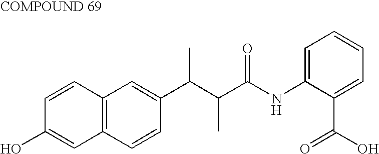 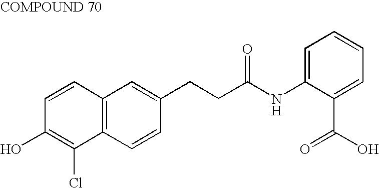 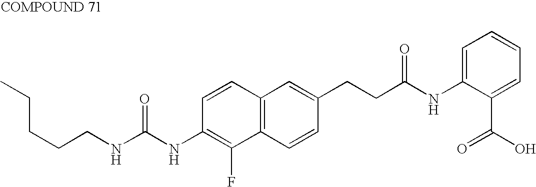 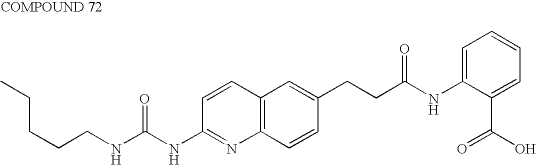 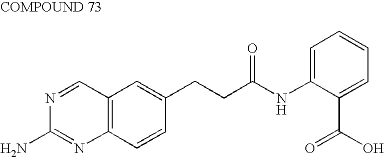 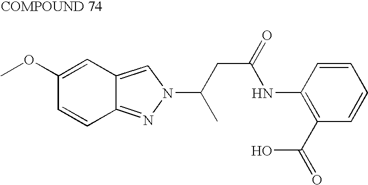 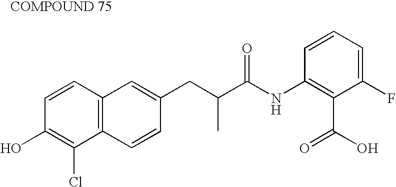 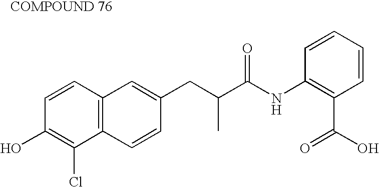 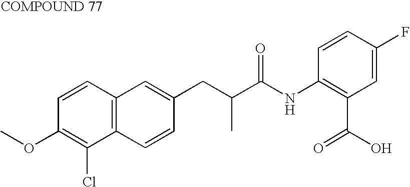 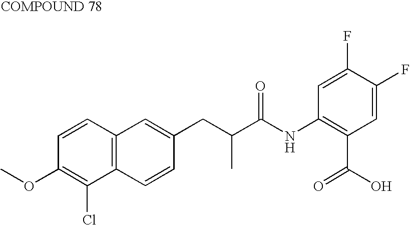 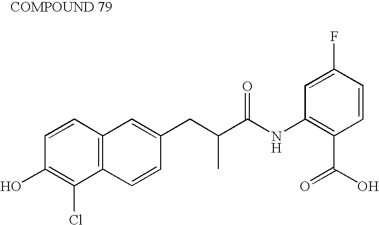 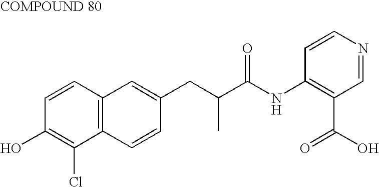 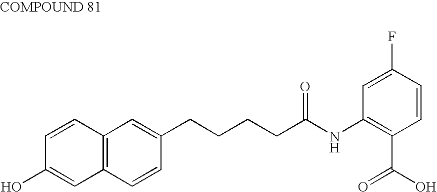 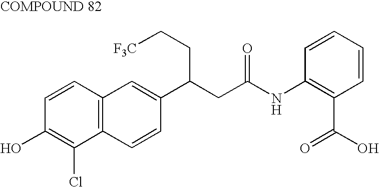 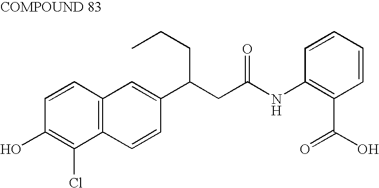 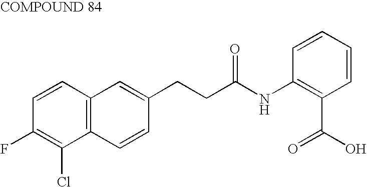 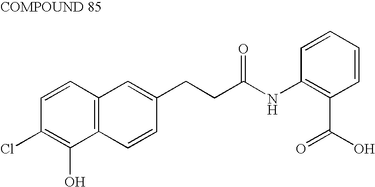 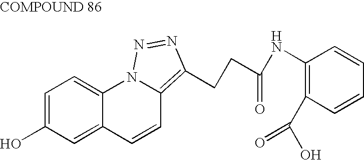 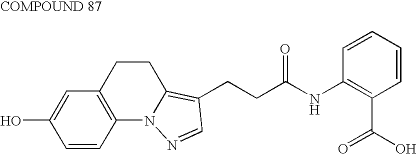 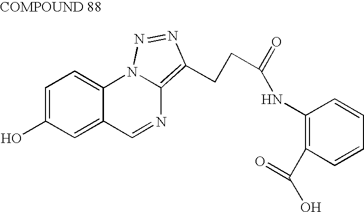 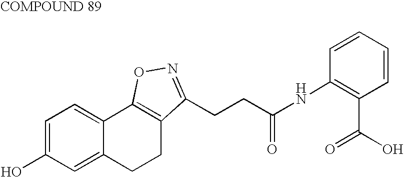 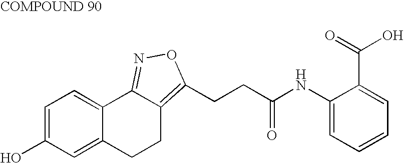 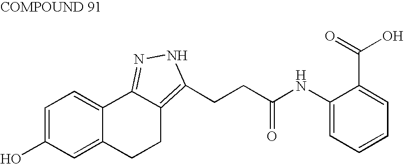 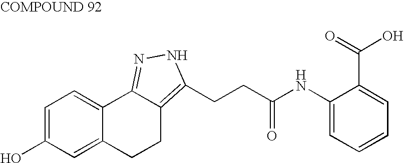 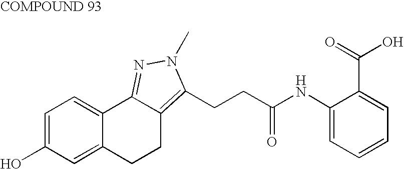 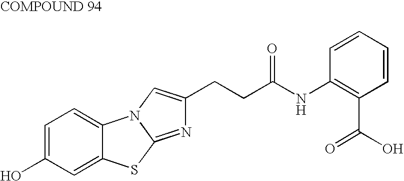 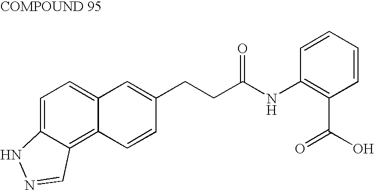
or a pharmaceutically acceptable salt or solvate thereof.

28. A pharmaceutical composition comprising a compound in accordance with any preceeding claim in combination with a pharmaceutically acceptable carrier.
